SEQUENCE LISTING (3 sequences)

SEQ ID NO: 1 - protein, 352 aa, Artificial Sequence
  Alvi pompejana MacroD2 (gp30765.t1)
MLTKRILAGRRLMSANRHRPIPDRICSLRRQSRATFSSQSDRGSDSLIPRVFRTIVGWFAAHLPRRGEGNGLGLDEDEKDALNVNSQHAKKKQKWQIEKEEYLKMPISEKRKIYKTQYVTLDSVPTWPEYYKKNLEASKVKPSKEPVDEALNSKVSLWCGDITTLEIDCIVNAANESLLGGGGVDGAIHRAAGPTLLAECRTACGCKTGDAKITGGYRLPAKYIIHTVGPRYHGDEKLRSCYKKSLDLMIENNLHSIAFPCISTGIYGFPGERAADIALTTVKDFLQKHKDKVDRIIFCLFTRDDVNIYESKMPTYFPVEGQCSDHEDQTTDDNPNVDLPGKPSDKNSVPKL

SEQ ID NO: 2 - protein, 506 aa, Artificial Sequence
  Alvi pompejana GDAP2 (g72977.t1)
MDDPLAAPTQATVDHTKLVRWNQTNVPEYNMVIDNPDDKKKSPFAYNREINQKIVLWSGDITALDTEAILHSTNETLSDVYPASERLLKRAGPDLQKDLSSNVKVCRTGEARLTKGYQLPARYVIHTVGPRYNLKYKTAAESALFNSYRSVLQIVREKQMSSVALCCIHASRRGYPPQEGAHIALRTVRRFLEKYGDTIDTVVFVVTGEDEDVYISLLPLYFPRSEHEEDFAAYQLPDDVGNEDGEPVIKERQIRIMGKPAYEAQKFNWKSPEELEESININEAFDTSIAVGAHSFSKMDDDIDKKRRIRLQYETHIALLNKEQYKRYEKWLKRSYQEDLSPMESLRCLYQSGFDVYGRPVVVFIGRHFPATKIDLDKFTLYLVQLMDNIVNKPYVIVYFHTLTQSDNHLDAGYLRSLYNLLDSRYKQNLGAVYVVHPTFWSKVMTWFFMTFNTTDLKSRIHNIPGLEYLFKRIPMDQLDIPDFISDYDIQVHGTRYYNPDVDKNL

SEQ ID NO: 3 - protein, 182 aa, Artificial Sequence
  Synthetic sequence
MHHHHHHLEVLFQGPVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFL

(19) United States
(12) Patent Application Publication
(10) Pub. No.: US 2024/0109892 A1
(43) Pub. Date: Apr. 4, 2024

(54) POLY(ADP-RIBOSE) GLYCOHYDROLASE (PARG) INHIBITORS AGAINST COVID MACRODOMAIN AND METHODS OF USING THE SAME

(71) Applicants: BOARD OF REGENTS, THE UNIVERSITY OF TEXAS SYSTEM, Austin, TX (US); BIOVENTURES, LLC, Little Rock, AR (US)

(72) Inventors: John A. TAINER, Houston, TX (US); Zamal AHMED, Sugarland, TX (US); Darin E. JONES, Little Rock, AR (US); Davide MOIANI, Houston, TX (US); Chris Arlen BROSEY, Houston, TX (US)

(21) Appl. No.: 18/262,682

(22) PCT Filed: Jan. 24, 2022
(86) PCT No.: PCT/US2022/013528

§ 371 Date: Jul. 24, 2023

Related U.S. Application Data
(60) Provisional application No. 63/140,912, filed on Jan. 24, 2021.

(51) Int. Cl. C07D 473/22; C07D 473/08

(52) U.S. Cl. CPC C07D 473/22; C07D 473/08

(57) ABSTRACT

The present disclosure provides compounds of Formula (Ia) and Formula (Ib) and the pharmaceutically acceptable salts and solvates thereof, wherein A¹, A², R¹, R², R³, and R⁴ are defined as set forth in the specification. The present disclosure also provides the use of compounds of Formula (Ia) or Formula (Ib) to treat a coronavirus infection in a subject.

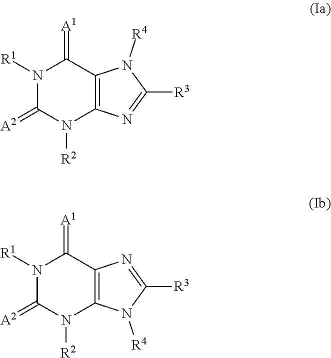

### GOVERNMENTAL RIGHTS

This invention was made with government support under grant number CA200231 awarded by the National Institutes of Health. The government has certain rights in the invention.

### INCORPORATION BY REFERENCE OF SEQUENCE LISTING

The content of the electronically submitted sequence listing (Name: 4443_004PC01_Seglisting_ST25.txt; Size: 9,564 Bytes; and Date of Creation: Jan. 24, 2022) is herein incorporated by reference in its entirety.

### BACKGROUND

PARG has been the focus of concentrated inhibitor development for cancer, both as a complement to and substitute for clinical PARP inhibitors (PARPi) (Chen and Yu, 2019; Houl et al., 2019; Slade, 2020), which act in part by trapping PARP1 on damaged DNA (Zandarashvili et al., 2020) and by acting synergistically to kill cancer cells with defective homology-directed repair (Syed and Tainer, 2018) or alternative end joining (Eckelmann et al., 2020). Following DNA damage, PARG reverses the signaling response initiated by PARP1 at ssDNA breaks by hydrolyzing the ‘cloud’ of poly(ADP-ribose) (PAR) into mono-nucleotide ADP-ribose (ADPr) (Pourfarjam et al., 2020; Slade et al., 2011). The dispersion of the PAR cloud enables subsequent progression of DNA repair at the damage site. Excessive and unresolved PAR depletes NAD⁺ and triggers cell death by parthanatos (Brosey et al., 2016; Wang et al., 2011). PARG is a member of the macrodomain family and shares a conserved fold for recognition and chemical hydrolysis of ADP-ribose moieties with CoV-2 Mac1 (Kim et al., 2012; Lambrecht et al., 2015; Rack et al., 2020a).

The largest SARS-CoV-2 non-structural protein, Nsp3, contains three macrodomain folds: Mac1 and two SUD-M-like domains (SUD-M-N, SUD-M-C) (Alhammad and Fehr, 2020; Frick et al., 2020). The CoV-2 Mac1 domain possesses mono(ADP-ribosyl) hydrolase activity in vitro, reversing PARP14 modifications (Rack et al., 2020b) and is proposed to remove single ADP-ribose modifications from host protein substrates in cells (Alhammad and Fehr, 2020; Lin et al., 2020; Rack et al., 2020b). Catalytic inactivation of coronavirus macrodomains attenuates viral pathogenesis in mice and restores interferon responses (Abraham et al., 2020; Fehr et al., 2015; Fehr et al., 2016; McPherson et al., 2017). Viral macrodomains are believed to counter or hijack host immunity by reversing the mono(ADP-ribosyl) modifications generated by host PARP14 enzymes, thereby interfering with interferon production (Grunewald et al., 2019) and altering STAT1 regulation (Iwata et al., 2016), a possible link to the damaging and deadly Cytokine Storm Syndrome observed in severe COVID-19 cases (Claverie, 2020). Screening efforts are underway to discover fragment binders (Fraser, 2020).

There is a need for anti-viral therapies to combat current and emerging viruses.

### BRIEF DESCRIPTION OF THE DRAWINGS

FIG. 1A is two graphs showing the binding affinity of ADP-ribose to purified CoV-2 Mac1.

FIG. 1B is two representative images of the crystal structure of CoV-2 Mac1 with a MES buffer molecule occupying the distal ribose binding site. MES hydrogen bonds with conserved N40 and Loop 2 main chain atoms.

FIG. 2A is a representative image of the phylogenetic tree of representative macrodomains forms six subfamilies. The CoV-2 Mac1 domain (indicated by an asterisk) is associated with the MacroD subfamily and shares closest homology with the N-terminal macrodomains of PARP9 and PARP14.

FIG. 2B is two representative images of the CoV-2 Mac1 Marcro-link domain. Domain and active site organization of CoV-2 Mac1 (PDB: 6W02) and PARG (PDB: 4B1H). The CoV-2 Mac1 domain forms a compact MacroD-like fold with Loops 1 and 2 coordinating the distal ribose through main chain hydrogen bonds. The macrodomain core of PARG is framed by an N-terminal accessory domain (teal) and helical C-terminus (gold), while a 0-hairpin loop insert into the macrodomain wraps around to the active site to form a ‘tyrosine clasp’ (Y795, purple). Residues that contact ADP-ribose are highlighted in orange with hydrogen bonds (blue dashed lines) to the ADP-ribose ligand (teal).

FIG. 3A is a representative image of protein-specific ET analysis prioritizes CoV-2 Mac1 and PARG active sites (red/orange—high, green—low) as the location of greatest functional importance within the full-length proteins.

FIG. 3B is a bar graph of ET values (100—most important, 0—unimportant) against the CoV-2 sequence reveals that adenosyl residue F156 is not as highly conserved among viral macrodomains.

FIG. 3C is a representative image of ET values calculated across the entire macrodomain family highlight active site residues with the greatest shared functional conservation between CoV-2 (F156, D22, N40, F132) and PARG (F902, E727, N740, F875).

FIG. 4A is two representative images of JA2131 PARGi inhibitor binding compactly in the adenosyl pocket of the active site, compared to the elongated conformation of ADP-ribose.

FIG. 4B is six representative images of crystal structures of PARG/inhibitor complexes from JA2131 derivatives modifying the C8 (PARG-002, PARG-061, PARG-292, PARG-322) or N7 (PARG-119, PARG-131) positions.

FIG. 5A is two representative images of PARGi poses associated into clusters anchored in the adenosyl pocket (left) or distal ribose pocket (right) of the CoV-2 Mac1 active site.

FIG. 5B is six representative images of PARGi poses from in silico screening show hydrogen bond contacts (solid lines) with N40, the Loop 2 main chain, or other residues (cartoon).

FIG. 5C is two representative images of the computational model of CoV-2 Mac1 inhibitor MES8-4 shows the ligand bridging the adenosyl and distal ribose binding sites and making hydrogen bond contacts with conserved N40, the Loop 2 main chain, and 123 main chain.

FIG. 6A is two representative images of the crystal structure of the Mac1/PARG-345 (MES8-4) complex. In addition to a main-chain contact (I23) at the xanthine head, the PARG-345 morpholine engages the critical N40 side-chain and makes direct backbone contacts with Loop 2 through the sulfonyl linker.

FIG. 6B is two representative image of the crystal structure of the Mac1/PARG-329 complex. The thiourea of PARG-329 engages Loop 2 through water-mediated contacts and adopts a strained morpholine conformation to fit into the active site.

FIG. 6C is two representative images depicting an overlay of the original MES8-4 computational model with the crystallized PARG-329 and PARG-345 ligands. MES8-4 successfully predicts the binding path and key contacts of these extended ligands in the Mac1 active site.

FIG. 7 is an image of purified CoV-2 Mac1 protein.

FIG. 8 is six polder maps of PARGi ligands. Maps are contoured to 36.

FIG. 9A is two representative images of computational docking of ADP-ribose (right) captures the binding path of the crystallized ligand (left).

FIG. 9B is two representative images of top ranked poses from MES8-2 (left) and MES8-4 models (right).

FIG. 10A is three graphs showing the MST measurements of the PARG-329 interaction with Mac1.

FIG. 10B is two graphs showing the ITC measurements of the PARG-239 interaction with Mac1.

FIG. 10C is a polder map of PARG-329, contoured to 36.

FIG. 11A is three graphs showing the MST measurements of the PARG-345 interaction with Mac1

FIG. 11B is two graphs showing the ITC measurements of the PARG-345 interaction with Mac1.

FIG. 11C is a polder map of PARG-345, contoured to 36.

### SUMMARY

The present disclosure provides a method for treating or reducing the pathogenicity and symptoms associated with a coronavirus infection, the method comprising administering to a subject in need thereof a therapeutically effective amount of a compound of Formula (Ia) or Formula (Ib):

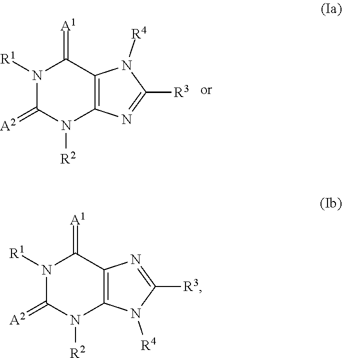

wherein:
A¹ and A² are independently O, S, or NH;
R¹ and R² are independently unsubstituted C₁-C₆ alkyl, (hydroxy)C₁-C₆ alkyl, or (C₃-C₆ cycloalkyl)C₁-C₆ alkyl;
R³ is H or —X—Y—R⁵;
R⁴ is H or —Y′—R^{5′};
X is absent, S, O, or NH;
Y is —(CH₂)ₙ— or —(CH₂)ₘ(C═O)—;
n is an integer of 1, 2, 3, 4, 5, or 6;
m is an integer of 0, 1, 2, 3, 4, 5, or 6;
Y′ is —(CH₂)_{n′}— or —(CH₂)_{m′}(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo,

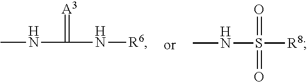

A³ is O, S, or NH;
R⁶ is optionally substituted C₁-C₆ alkyl, optionally substituted (3- to 10-membered heterocyclo)C₁-C₆ alkyl, or S(═O)₂R⁷;
R⁷ is optionally substituted C₁-C₆ alkyl or optionally substituted (3- to 10-membered heterocyclo)C₁-C₆ alkyl;
R⁸ is optionally substituted C₁-C₆ alkyl or optionally substituted (3- to 10-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo,

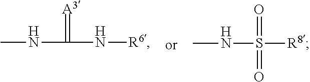

A³ is O, S, or NH;
R^{6′} is optionally substituted C₁-C₆ alkyl, optionally substituted (3- to 10-membered heterocyclo)C₁-C₆ alkyl, or S(═O)₂R^{7′};
R⁷ is optionally substituted C₁-C₆ alkyl or optionally substituted (3- to 10-membered heterocyclo)C₁-C₆ alkyl;
R^{8′} is optionally substituted C₁-C₆ alkyl or optionally substituted (3- to 10-membered heterocyclo)C₁-C₆ alkyl; and
or a pharmaceutically acceptable salt thereof,
   with the proviso that R³ and R⁴ are not both H.

In some aspects, the method is directed to treating a coronavirus infection, the method comprising administering to a subject in need thereof a therapeutically effective amount of a compound of Formula (Ia) or Formula (Ib).

In some aspects, the method is directed to reducing the symptoms associated with a coronavirus infection, the method comprising administering to a subject in need thereof a therapeutically effective amount of a compound of Formula (Ia) or Formula (Ib).

In some aspects, the method is directed to reducing the pathogenicity and symptoms associated with a coronavirus infection, the method comprising administering to a subject in need thereof a therapeutically effective amount of a compound of Formula (Ia) or Formula (Ib).

In some aspects, the present disclosure provides a compound of Formula (Ia) or Formula (Ib), as defined herein, for the manufacture of a medicament to treat or reduce the symptoms associated with a coronavirus infection in a subject in need thereof.

In some aspects, the present disclosure provides a compound of Formula (Ia) or Formula (Ib), as defined herein, for use to treat or reduce the symptoms associated with a coronavirus infection in a subject in need thereof.

In some aspects, the present disclosure provides use of a compound of Formula (Ia) or Formula (Ib), as defined herein, to treat or reduce the symptoms associated with a coronavirus infection in a subject in need thereof.

In some aspects, the present disclosure provides a compound of Formula (Ia) or Formula (Ib):

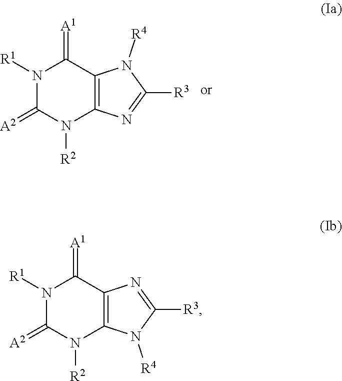

wherein:
A¹ and A² are independently O, S, or NH;
R¹ and R² are independently unsubstituted C₁-C₆ alkyl, (hydroxy)C₁-C₆ alkyl, or (C₃-C₆ cycloalkyl)C₁-C₆ alkyl; and
R³ is H or —X—Y—R⁵;
R⁴ is H or —Y′—R^{5′};
X is absent, S, O, or NH;
Y is —(CH₂)ₙ— or —(CH₂)ₘ(C═O)—;
n is an integer of 1, 2, 3, 4, 5, or 6;
m is an integer of 0, 1, 2, 3, 4, 5, or 6;
Y′ is —(CH₂)ₙ— or —(CH₂)ₘ(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo,

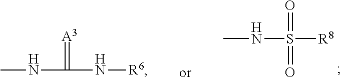

A³ is O, S, or NH;
R⁶ is optionally substituted C₁-C₆ alkyl or S(═O)₂R⁷;
R⁷ is optionally substituted C₁-C₆ alkyl, or optionally substituted (3- to 10-membered heterocyclo)C₁-C₆ alkyl;
R⁸ is optionally substituted (3- to 6-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo,

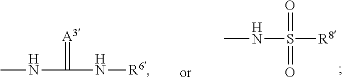

A³ is O, S, or NH;
R^{6′} is optionally substituted C₁-C₆ alkyl or S(═O)₂R⁷;
R^{7′} is optionally substituted C₁-C₆ alkyl, or optionally substituted (3- to 10-membered heterocyclo)C₁-C₆ alkyl; and
R^{8′} is optionally substituted (3- to 6-membered heterocyclo)C₁-C₆ alkyl;
or a pharmaceutically acceptable salt thereof, with the proviso that R³ and R⁴ are not both H.

In some aspects, the present disclosure provides a method for treating or reducing the symptoms associated with a coronavirus infection. In some aspects, the method comprising administering to a subject in need thereof a therapeutically effective amount of a compound of Formula (Ia) or Formula (Ib), as disclosed herein, or any composition comprising Formula (Ia) or Formula (Ib), as disclosed herein.

In some aspects, the present disclosure provides a method for inhibiting coronavirus pathogenicty and symptoms by administering to a subject in need thereof a therapeutically effective amount of a compound that is chemically complementary to the Mac1 channel and interactions defined and implied by the crystal structures of the Mac1/PARG-345 (MES8-4) complex and the Mac1/PARG-329 complex with their neighboring bound water molecules.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about,” as used herein, includes the recited number ±10%. Thus, “about 10” means 9 to 11.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one aspect, the alkyl is a C₁-C₁₀ alkyl. In some aspects, the alkyl is a C₁-C₆ alkyl. In some aspects, the alkyl is a C₁-C₄ alkyl. In some aspects, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy (i.e., —C(═O)OH), alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, amino, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted heteroaryl, (C₃-C₆ cycloalkyl)oxy, or (4- to 8-membered heterocyclo)oxy;
R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl;
R⁶⁰ is selected from the group consisting of cyano, nitro, hydroxy, C₁-C₆ alkoxy, —C(═O)R⁶², and —S(═O)₂R⁶²;
R⁶¹ is selected from the group consisting of C₁-C₆ alkyl, C₃-C₆ cycloalkyl, and —NR^{63a}R^{63b};
R⁶² is selected from the group consisting of C₁-C₆ alkyl, C₃-C₆ cycloalkyl, and —NR^{63a}R^{63b};
R^{63a} is selected from the group consisting of hydrogen, C₁-C₆ alkyl, and C₃-C₆ cycloalkyl;
R^{63b} is selected from the group consisting of hydrogen, C₁-C₆ alkyl, and C₃-C₆ cycloalkyl; or
R^{63a} and R^{63b} taken together with the nitrogen atom to which they are attached form a 4- to 6-membered optionally substituted heterocyclo;
R⁶⁴ is selected from the group consisting of C₁-C₆ alkyl, C₃-C₆ cycloalkyl, and —NR^{63c}R^{63d}; and
R^{63c} is selected from the group consisting of hydrogen, C₁-C₆ alkyl, and C₃-C₆ cycloalkyl;
R^{63d} is selected from the group consisting of hydrogen, C₁-C₆ alkyl, and C₃-C₆ cycloalkyl; or
R^{63c} and R^{63d} taken together with the nitrogen atom to which they are attached form a 4- to 6-membered optionally substituted heterocyclo.

In some aspects, the optionally substituted alkyl is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a} C(═O)R^{56b}, —N(R^{56c})S(═O)₂R^{56d}, —C(═O)R⁵⁷, —S(═O)R^{56e}, or —S(═O)₂R⁵⁸.

In some aspects, the optionally substituted alkyl is substituted with two substituents. In some aspects, the optionally substituted alkyl is substituted with one substituent. In some aspects, the optionally substituted alkyl is an optionally substituted C₁-C₆ alkyl. In some aspects, the optionally substituted alkyl is an optionally substituted C₁-C₄ alkyl. In one aspect, the optionally substituted alkyl is an optionally substituted is a C₁ or C₂ alkyl. Non-limiting exemplary optionally substituted alkyl groups include —CH(CO₂Me)CH₂CO₂Me and —CH(CH₃)CH₂N(H)C(═O)O(CH₃)₃.

The terms “hydroxyalkyl” or “(hydroxy)alkyl” as used herein by themselves or as part of another group refer to an alkyl group substituted with one, two, or three hydroxy groups. In one aspect, the alkyl is a C₁-C₆ alkyl. In some aspects, the alkyl is a C₁-C₄ alkyl. In some aspects, the alkyl is a C₁ or C₂ alkyl. In some aspects, the hydroxyalkyl is a monohydroxyalkyl group, i.e., substituted with one hydroxy group. In some aspects, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “haloalkyl” as used herein by itself or as part of another group refers to an alkyl substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl and the resulting haloalkyl is thus referred to as a “C₁-C₆ haloalkyl.” In another embodiment, the alkyl is a C₁-C₄ alkyl and the resulting haloalkyl is thus referred to as a “C₁-C₄ haloalkyl.” In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “amino” as used by itself or as part of another group refers to a radical of the formula —NR^{55a}R^{55b}, wherein R^{55a} and R^{55b} are independently hydrogen, alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl. In one embodiment, the amino is —NH₂. In another embodiment, the amino is —N(H)CH₃ and —N(H)CH₂CH₃.

The term “alkoxy” as used herein by itself or as part of another group refers to an alkyl attached to a terminal oxygen atom. In one embodiment, the alkyl is a C₁-C₆ alkyl. In another embodiment, the alkyl is a C₁-C₄ alkyl group and thus the resulting alkoxy is referred to as a “C₁-C₄ alkoxy.” Non-limiting exemplary alkoxy groups include methoxy, ethoxy, and tert-butoxy.

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one aspect, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In some aspects, the heterocyclo group is a 5- to 8-membered cyclic group containing one ring and one or two nitrogen atoms and, optionally, one —CH₂— group is replaced with one —C(═O)— group. In some aspects, the heterocyclo group is a 5- or 6-membered cyclic group containing one ring and one or two nitrogen atoms and, optionally, one —CH₂— group is replaced with one —C(═O)— group. In some aspects, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

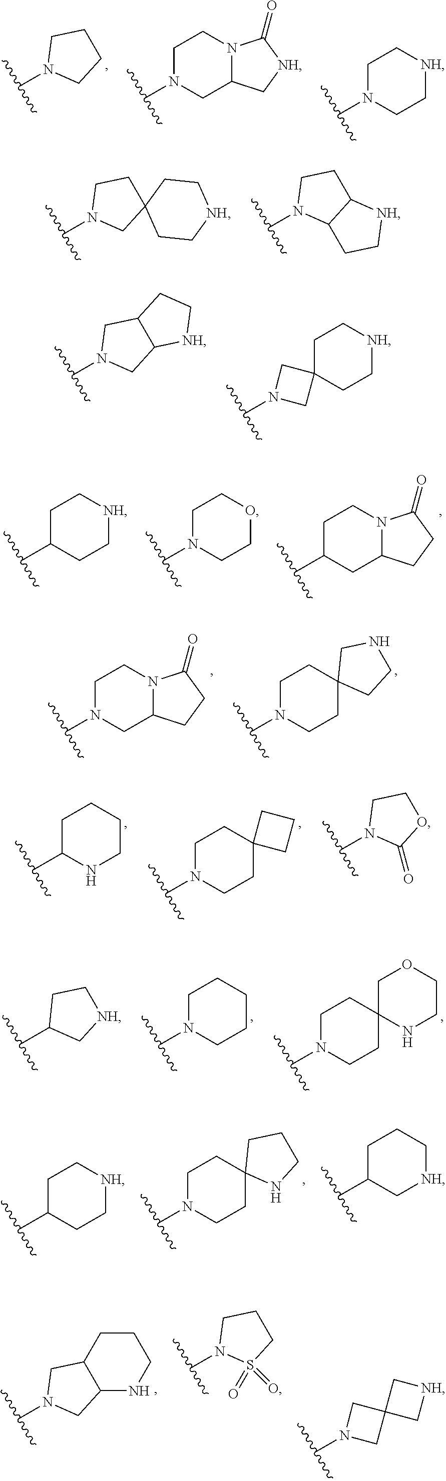

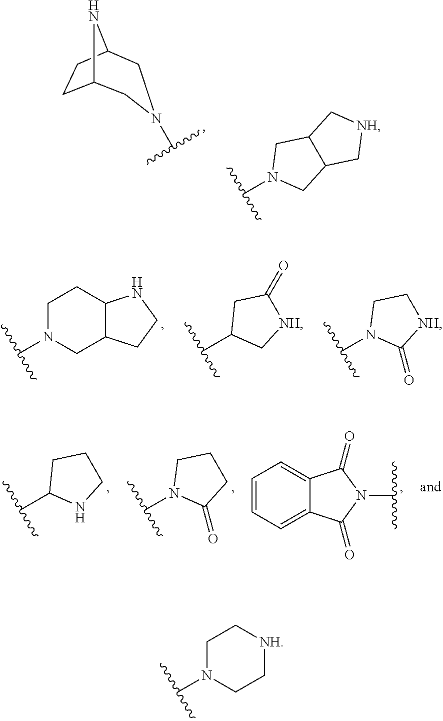

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkyl amino, or (heterocyclo)alkyl amino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56a}, —C(═O)R⁵⁷, —S(═O)R^{56e}, —S(═O)₂R⁵¹, —OR⁵⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴ wherein R^{56a}, R^{56b}, R^{56c}, R^{56d}, R^{56e}, R⁵⁷, R⁵⁸, R⁵⁹, R⁶⁰, R⁶¹ and R⁶⁴ are as defined in connection with the term “optionally substituted cycloalkyl.” Substitution may occur on any available carbon or nitrogen atom of the heterocyclo group. In one aspect, the optionally substituted heterocyclo is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹.

Non-limiting exemplary optionally substituted heterocyclo groups include:

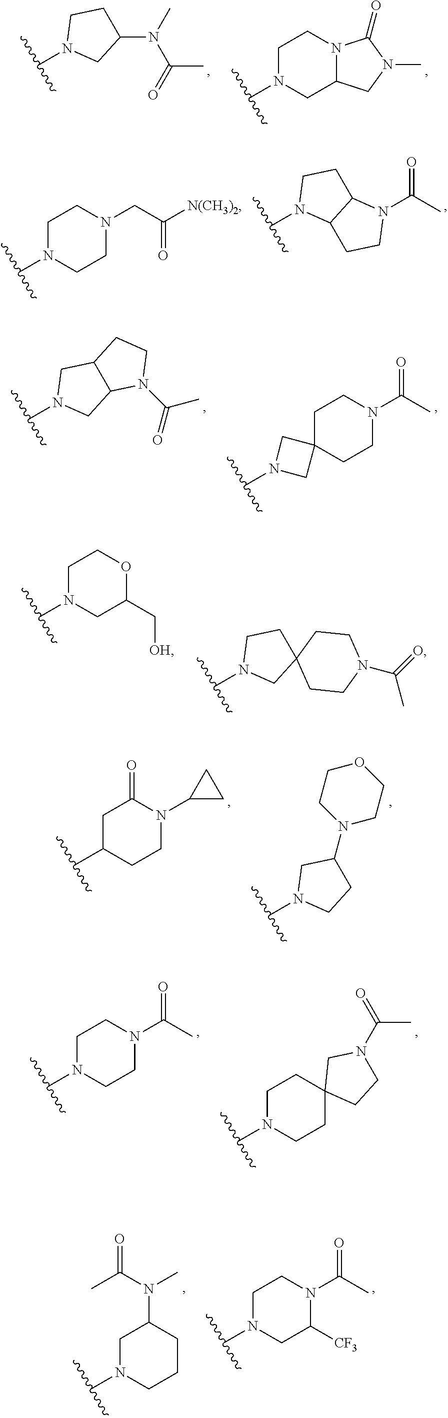

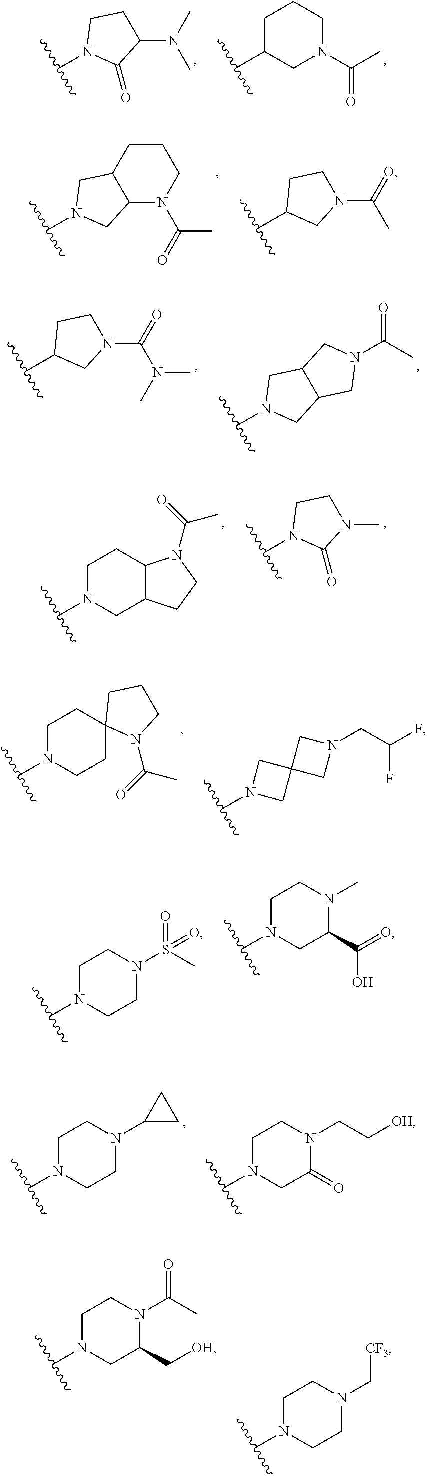

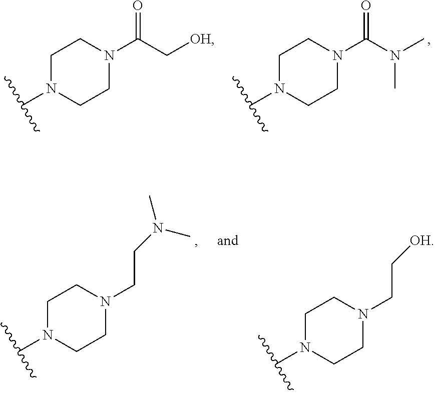 [SMILES: *N1CCN(C(=O)CO)CC1 ; *N1CCN(C(=O)N(C)C)CC1 ; *N1CCN(CCN(C)C)CC1 ; *N1CCN(CCO)CC1]

The term heterocycloalkyl or “(heterocyclo)alkyl” as used herein by themselves or as part of another group refer to an alkyl group substituted with a heterocyclo group. In one aspect, the alkyl is a C₁-C₆ alkyl. In some aspects, the alkyl is a C₁-C₄ alkyl. In some aspects, the alkyl is a C₁ or C₂ alkyl. Non-limiting exemplary “(heterocyclo)alkyl groups include 4-ethylmorpholine and 4-ethylthiomorpholine 1,1-dioxide.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one aspect, the aryl group is phenyl or naphthyl. In some aspects, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, —OR⁹, —N(R^{56a})C(═N—R⁶⁰)R⁶¹, —N(R^{56a})C(═C—NO₂)R⁶⁴, —C(═N—R⁶⁰)R⁶¹, or —C(═C—NO₂)R⁶⁴; wherein R^{56a}, R^{56b}, R^{56c}, R^{56d}, R^{56e}, R⁵⁷, R⁵⁸, R⁵⁹, R⁶⁰, R⁶¹, and R⁶⁴ are as defined in connection with the term “optionally substituted cycloalkyl.” In one aspect, the optionally substituted aryl is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹.

In one aspect, the optionally substituted aryl is an optionally substituted phenyl. In some aspects, the optionally substituted phenyl has four substituents. In some aspects, the optionally substituted phenyl has three substituents. In some aspects, the optionally substituted phenyl has two substituents. In some aspects, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of Formula (Ia) or Formula (Ib) to an individual in need of such treatment.

Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to an individual in need thereof.

The term “pathogenicity” as used herein refers to the ability of an organism (e.g., a virus) to cause disease or harm the host.

In the case of an infectious disease caused by a coronavirus, in one embodiment, a therapeutically effective amount will refer to the amount of a compound of Formula (Ia) or Formula (Ib) that causes a therapeutic response, e.g., decrease viral replication and/or infection in subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

### Methods of Treatment

In certain aspects, the disclosure provides a method for treating or reducing the symptoms associated with a coronavirus infection, the method comprising administering to a subject in need thereof a therapeutically effective amount of a compound of Formula (Ia) or Formula (Ib):

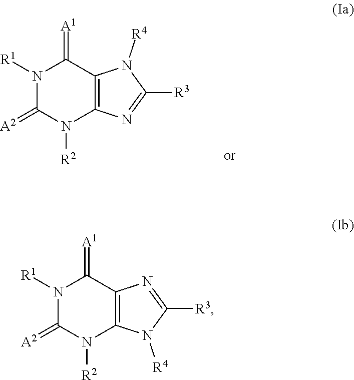

wherein:
A¹ and A² are independently 0, S, or NH;
R¹ and R² are independently unsubstituted C₁-C₆ alkyl, (hydroxy)C₁-C₆ alkyl, or (C₃-C₆ cycloalkyl)C₁-C₆ alkyl;
R³ is H or —X—Y—R⁵;
R⁴ is H or —Y′—R^{5′};
X is absent, S, O, or NH;
Y is —(CH₂)_{n′}— or —(CH₂)ₘ(C═O)—;
n is an integer of 1, 2, 3, 4, 5, or 6;
m is an integer of 0, 1, 2, 3, 4, 5, or 6;
Y′ is —(CH₂)_{n′}— or —(CH₂)_{m′}(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo,

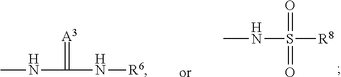

A³ is O, S, or NH;
R⁶ is optionally substituted C₁-C₆ alkyl, optionally substituted (3- to 10-membered heterocyclo)C₁-C₆ alkyl, or S(═O)₂R⁷;
R⁷ is optionally substituted C₁-C₆ alkyl or optionally substituted (3- to 10-membered heterocyclo)C₁-C₆ alkyl;
R⁸ is optionally substituted C₁-C₆ alkyl or optionally substituted (3- to 10-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo,

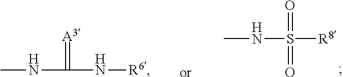

A³ is O, S, or NH;
R^{6′} is optionally substituted C₁-C₆ alkyl, optionally substituted (3- to 10-membered heterocyclo)C₁-C₆ alkyl, or S(═O)₂R^{7′};
R⁷ is optionally substituted C₁-C₆ alkyl or optionally substituted (3- to 10-membered heterocyclo)C₁-C₆ alkyl; and
R⁸ is optionally substituted C₁-C₆ alkyl or optionally substituted (3- to 10-membered heterocyclo)C₁-C₆ alkyl;
or a pharmaceutically acceptable salt thereof,
   with the proviso that R³ and R⁴ are not both H.

In some aspects, the methods of the disclosure comprise administering a compound of Formula (Ia).

In some aspects, the methods of the disclosure comprise administering a compound of Formula (Ib).

In some aspects, methods of the disclosure comprise administering a compound of Formula (Ia) or Formula (Ib), wherein A¹ and A² are independently 0 or S.

In some aspects, methods of the disclosure comprise administering a compound of Formula (Ia) or Formula (Ib), wherein A¹ and A² are 0.

In some aspects, methods of the disclosure comprise administering a compound of Formula (Ia) or Formula (Ib), wherein R¹ and R² are independently —CH₃, hydroxy(CH₂)₂-, or methylcyclopropyl.

In some aspects, methods of the disclosure comprise administering a compound of Formula (Ia) or Formula (Ib), wherein R³ is H and R⁴ is —Y′—R⁵.

In some aspects, methods of the disclosure comprise administering a compound of Formula (Ia) or Formula (Ib), wherein:
R⁸ is optionally substituted aryl, optionally substituted 3- to 9-membered heterocyclo,

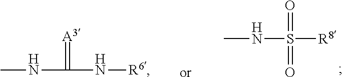

A^{3′} is O, S, or NH;
R^{6′} is optionally substituted C₁-C₆ alkyl or S(═O)₂R⁷; R⁷ is optionally substituted C₁-C₆ alkyl or optionally substituted (3- to 6-membered heterocyclo)C₁-C₆ alkyl; and
R⁸ is optionally substituted (3- to 6-membered heterocyclo)C₁-C₆ alkyl.

In some aspects, methods of the disclosure comprise administering a compound of Formula (Ia) or Formula (Ib), wherein R⁵ is C₁-C₄ haloalkyl, hydroxy, C₃-C₆ cycloalkyl, C₁-C₄ alkoxy, or amino.

In some aspects, methods of the disclosure comprise administering a compound of Formula (Ia) or Formula (Ib), wherein R⁵ is —OCH₃, —CF₃, hydroxy, —NH₂, —N(CH₃)₂, cyclopropyl, or cyclobutyl.

In some aspects, methods of the disclosure comprise administering a compound of Formula (Ia) or Formula (Ib), wherein:
n′ is 1 or 2;
R^{5′} is optionally substituted aryl, optionally substituted 9-membered heterocyclo,

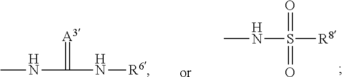

A³ is O, S, or NH;
R^{6′} is S(═O)₂R⁷;
R^{7′} is optionally substituted C₁-C₆ alkyl; and
R⁸ is optionally substituted (6-membered heterocyclo)C₁-C₆ alkyl.

In some aspects, methods of the disclosure comprise administering a compound of Formula (Ia) or Formula (Ib), wherein:
R^{5′} is

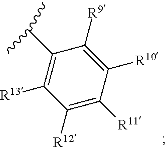

R^{9′}, R^{10′}, R^{11′}, R^{12′}, and R^{13′} are independently H, chloro, fluoro, C₁-C₆ alkyl, (CHO)C₁-C₆ alkyl,

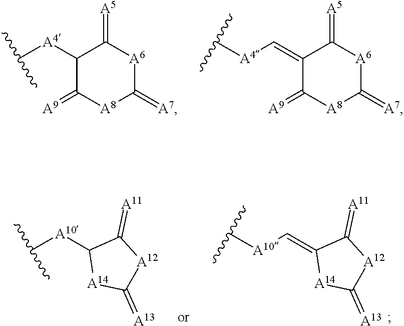

A⁴ is —(CH₂)ₚ—;
p is an integer of 1, 2, 3, 4, 5, or 6;
A⁴ is —(CH₂)_{q}—;
q is an integer of 1, 2, 3, 4, 5, or 6;
A⁵, A⁷, and A⁹ are independently O, S, or NH;
A⁶ and A⁸ are independently NH or CH₂;
A^{10′} is —(CH₂)ᵣ—;
r is an integer of 1, 2, 3, 4, 5, or 6;
A^{10″} is —(CH₂)ₜ—;
t is an integer of 1, 2, 3, 4, 5, or 6;
A¹¹ and A¹³ are independently O, S, or NH; and
A¹² and A¹⁴ are independently NH or CH₂.

In some aspects, methods of the disclosure comprise administering a compound of Formula (Ia) or Formula (Ib), wherein R⁴ is H and R³ is —X—Y—R⁵.

In some aspects, methods of the disclosure comprise administering a compound of Formula (Ia) or Formula (Ib), wherein:
R⁵ is optionally substituted aryl, optionally substituted 3- to 9-membered heterocyclo,

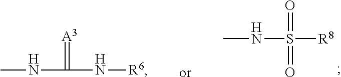

A³ is O, S, or NH;
R⁶ is optionally substituted C₁-C₆ alkyl or S(═O)₂R⁷;
R⁷ is optionally substituted C₁-C₆ alkyl or optionally substituted (3- to 6-membered heterocyclo)C₁-C₆ alkyl; and
R⁸ is optionally substituted (3- to 6-membered heterocyclo)C₁-C₆ alkyl.

In some aspects, methods of the disclosure comprise administering a compound of Formula (Ia) or Formula (Ib), wherein R⁵ is an optionally substituted 3- to 6-membered heterocyclo selected from the group consisting of:

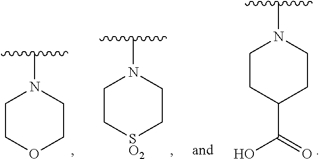

In some aspects, methods of the disclosure comprise administering a compound of Formula (Ia) or Formula (Ib), wherein:
X is S, O, or NH;
n is an integer of 1, 2, or 3;
m is an integer of 1 or 2;
R⁵ is optionally substituted aryl, optionally substituted 9-membered heterocyclo,

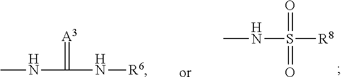

A³ is O;
R⁶ is S(═O)₂R⁷;
R⁷ is (morpholine)C₁-C₆ alkyl; and R⁸ is optionally substituted (6-membered heterocyclo) C₁-C₆ alkyl.

In some aspects, methods of the disclosure comprise administering a compound of Formula (Ia) or Formula (Ib), wherein:
Y is —(CH₂)ₘ(C═O)—;
m is 2;
R⁵ is:

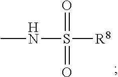

and
R⁸ is optionally substituted (piperazine) C₁-C₃ alkyl.

In some aspects, methods of the disclosure comprise administering a compound of Formula (Ia) or Formula (Ib), wherein R⁸ is:

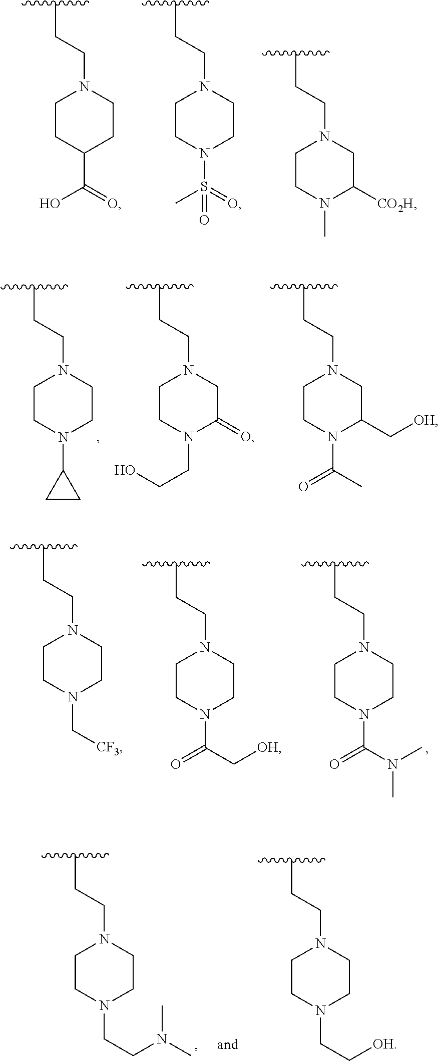

In some aspects, methods of the disclosure comprise administering a compound of Formula (Ia) or Formula (Ib), wherein:
R⁵ is:

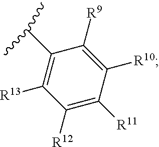

R⁹, R¹⁰, R¹¹, R¹², and R¹³ are independently H, chloro, fluoro, C₁-C₆ alkyl, or (CHO)C₁-C₆ alkyl.

In some aspects, methods of the disclosure comprise administering a compound of Formula (Ia) or Formula (Ib), wherein R³ is —X—Y—R⁵ and R⁴ is —Y′—R^{5′}.

In some aspects, methods of the disclosure comprise administering a compound of Formula (Ia) or Formula (Ib), wherein:
Y′ is —(CH₂)_{n′}—,
n′ is 1; and
R^{5′} is optionally substituted aryl.

In some aspects, methods of the disclosure comprise administering a compound of Formula (Ia) or Formula (Ib), wherein:
R⁵ is

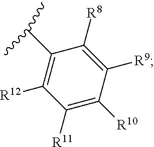

R⁸, R⁹, R¹⁰, R¹¹, and R¹² are independently H, chloro, fluoro, C₁-C₆ alkyl, or (CHO)C₁-C₆ alkyl.

In some aspects, methods of the disclosure comprise administering a compound of Formula (Ia) or Formula (Ib), wherein:
X is S;
Y is —(CH₂)ₙ—;
n is 2; and
R⁵ is optionally substituted 3- to 6-membered heterocyclo.

In some aspects, methods of the disclosure comprise administering a compound of Formula (Ia) or Formula (Ib), wherein R⁵ is selected from the group consisting of:

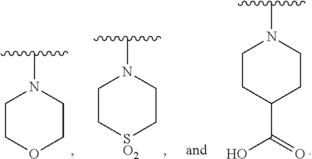

In some aspects, methods of the disclosure comprise administering a compound of Formula (Ia), wherein the compound of Formula (Ia) is selected from the group consisting of:

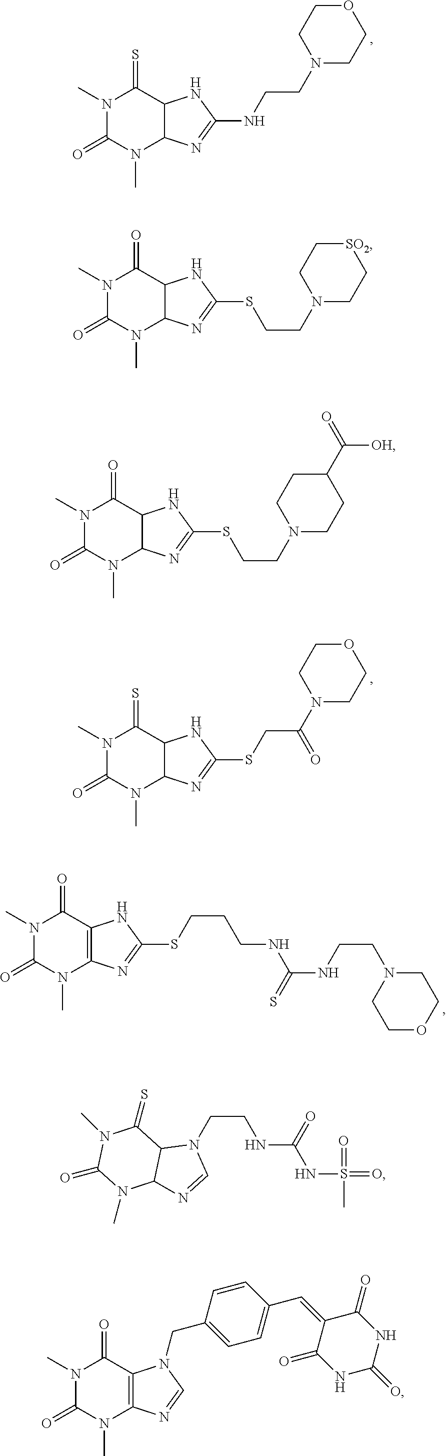

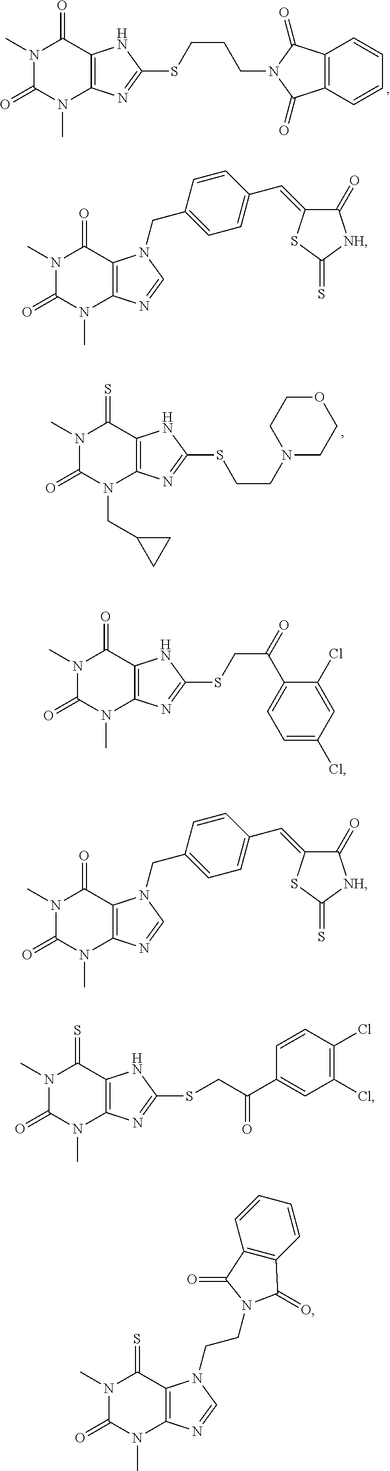

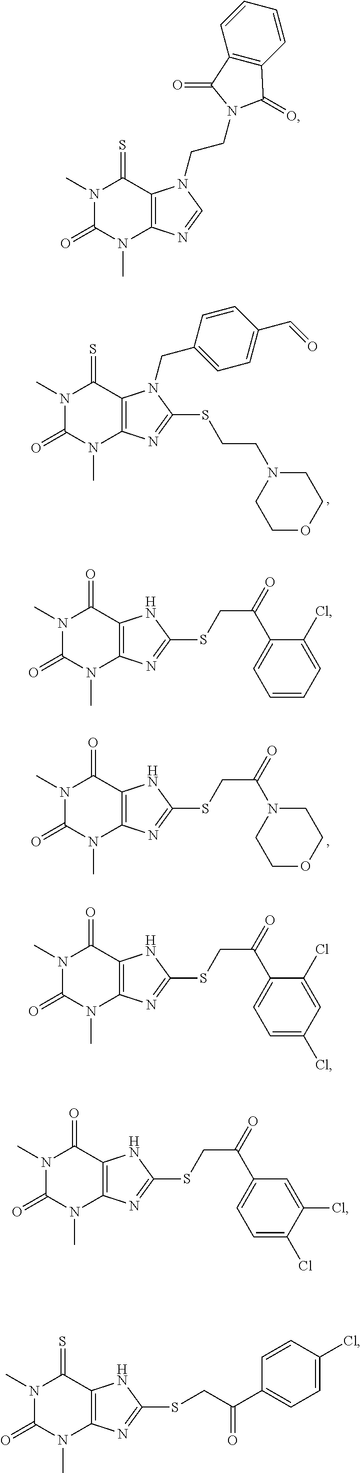

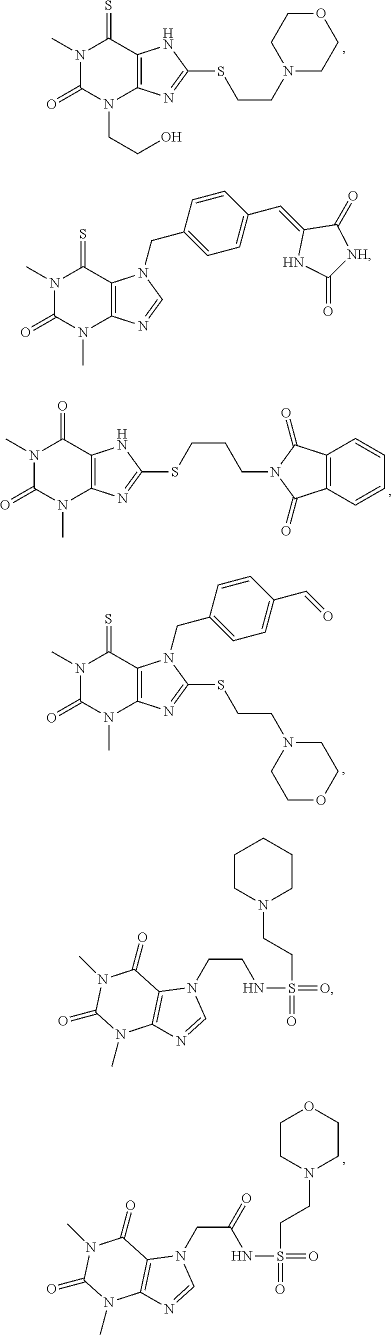

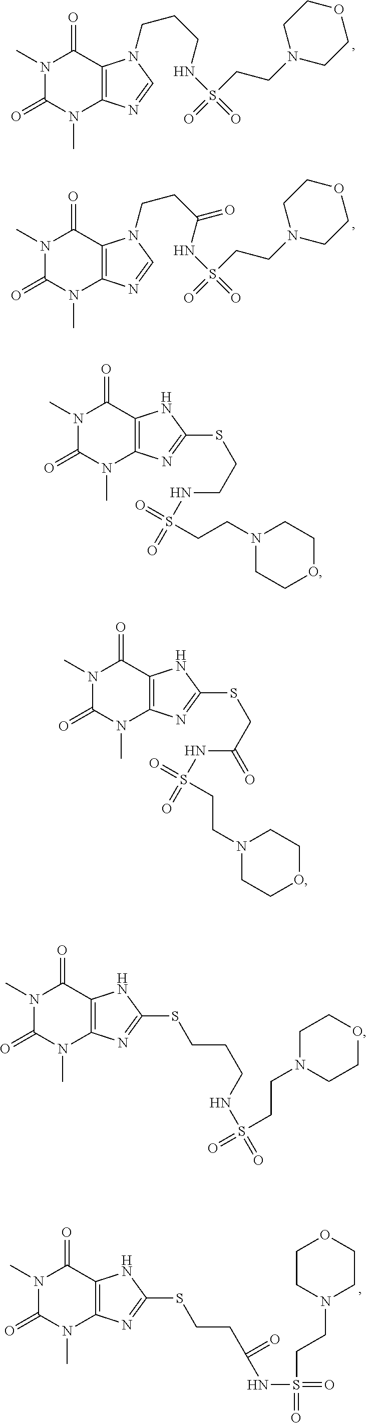 [SMILES: Cn1c(=O)c2c(ncn2CCCNS(=O)(=O)CCN2CCOCC2)n(C)c1=O ; Cn1c(=O)c2c(ncn2CCC(=O)NS(=O)(=O)CCN2CCOCC2)n(C)c1=O ; Cn1c(=O)c2[nH]c(SCCNS(=O)(=O)CCN3CCOCC3)nc2n(C)c1=O ; Cn1c(=O)c2[nH]c(SCC(=O)NS(=O)(=O)CCN3CCOCC3)nc2n(C)c1=O ; Cn1c(=O)c2[nH]c(SCCCNS(=O)(=O)CCN3CCOCC3)nc2n(C)c1=O ; Cn1c(=O)c2[nH]c(SCCC(=O)NS(=O)(=O)CCN3CCOCC3)nc2n(C)c1=O]

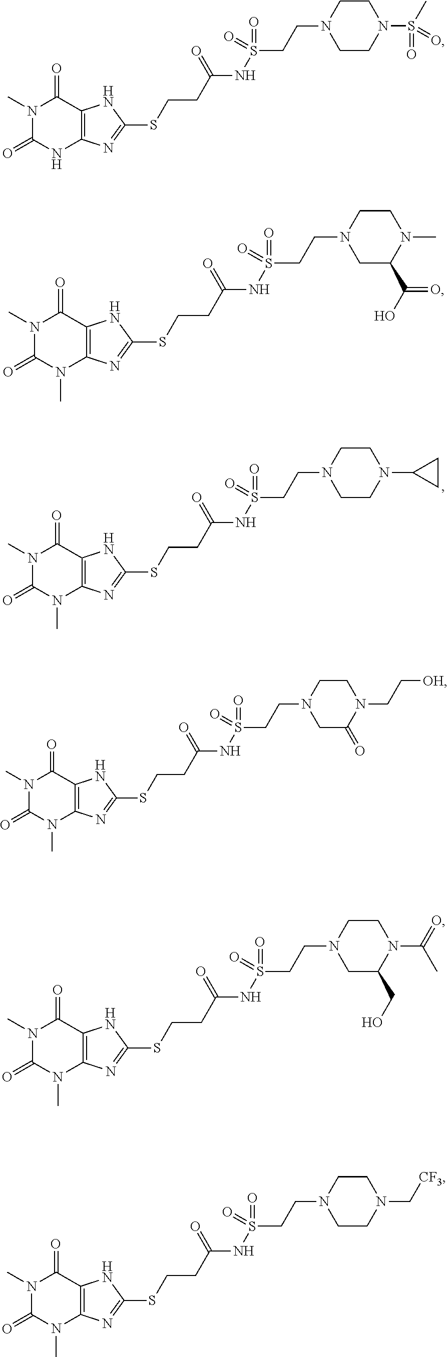 [SMILES: Cn1c(=O)[nH]c2nc(SCCC(=O)NS(=O)(=O)CCN3CCN(S(C)(=O)=O)CC3)[nH]c2c1=O ; CN1CCN(CCS(=O)(=O)NC(=O)CCSc2nc3c([nH]2)c(=O)n(C)c(=O)n3C)C[C@@H]1C(=O)O ; Cn1c(=O)c2[nH]c(SCCC(=O)NS(=O)(=O)CCN3CCN(C4CC4)CC3)nc2n(C)c1=O ; Cn1c(=O)c2[nH]c(SCCC(=O)NS(=O)(=O)CCN3CCN(CCO)C(=O)C3)nc2n(C)c1=O ; CC(=O)N1CCN(CCS(=O)(=O)NC(=O)CCSc2nc3c([nH]2)c(=O)n(C)c(=O)n3C)C[C@@H]1CO ; Cn1c(=O)c2[nH]c(SCCC(=O)NS(=O)(=O)CCN3CCN(CC(F)(F)F)CC3)nc2n(C)c1=O]

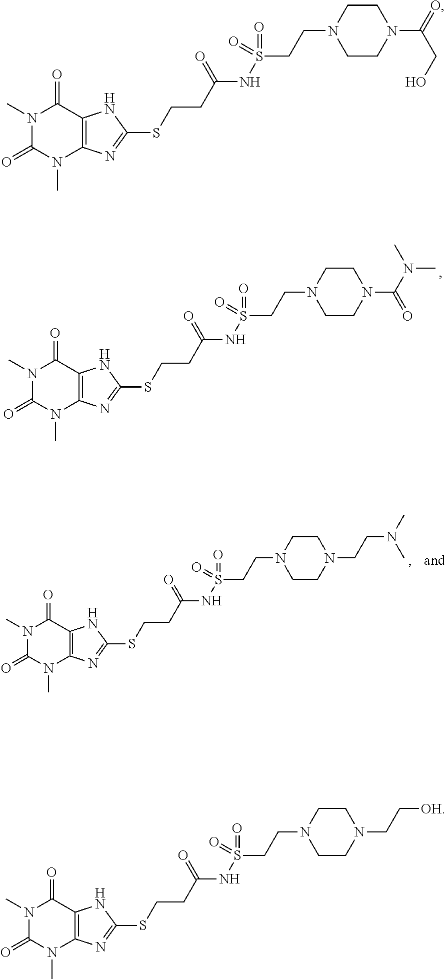 [SMILES: Cn1c(=O)c2[nH]c(SCCC(=O)NS(=O)(=O)CCN3CCN(C(=O)CO)CC3)nc2n(C)c1=O ; CN(C)C(=O)N1CCN(CCS(=O)(=O)NC(=O)CCSc2nc3c([nH]2)c(=O)n(C)c(=O)n3C)CC1 ; CN(C)CCN1CCN(CCS(=O)(=O)NC(=O)CCSc2nc3c([nH]2)c(=O)n(C)c(=O)n3C)CC1 ; Cn1c(=O)c2[nH]c(SCCC(=O)NS(=O)(=O)CCN3CCN(CCO)CC3)nc2n(C)c1=O]

In some aspects, methods of the disclosure comprise administering a compound of Formula (Ib), wherein the compound of Formula (Ib) is selected from the group consisting of:

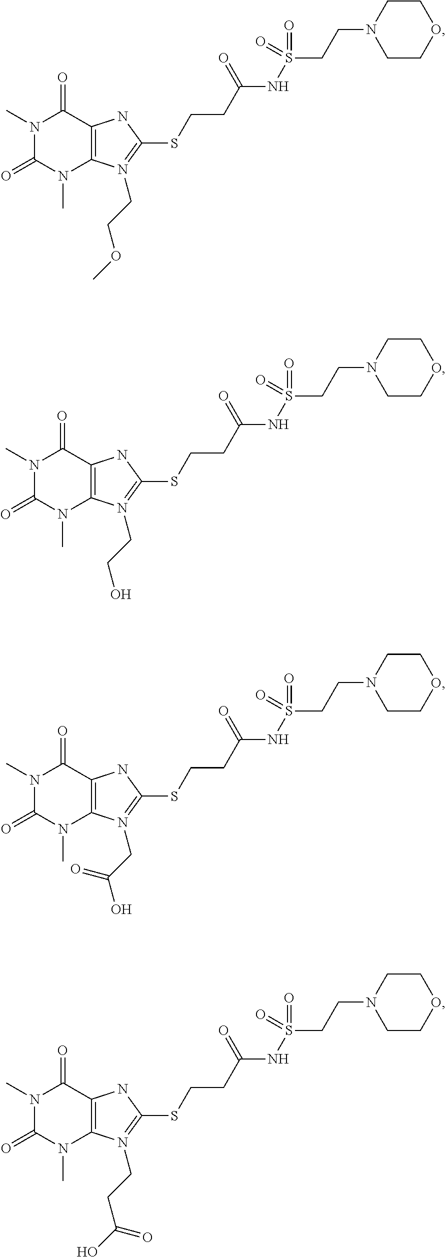

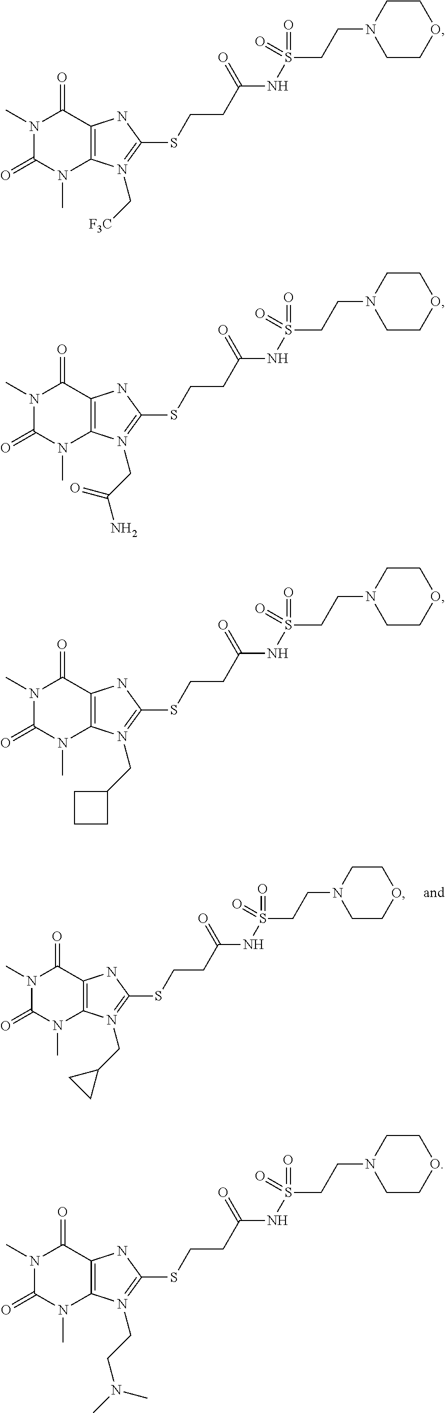

In some aspects, the coronavirus infection is caused by human coronavirus OC43 (HCoV-OC43), human coronavirus HKU1 (HCoV-HKU1), human coronavirus 229E (HCoV-229E), human coronavirus NL63 (HCoV-NL63), middle east respiratory syndrome-related coronavirus (MERS-CoV), severe acute respiratory syndrome coronavirus (SARS-CoV), or severe acute respiratory syndrome coronavirus 2 (SARS-CoV-2)

In some aspects, methods of the disclosure comprise administering a compound of Formula (Ia) or Formula (Ib), wherein the compound of Formula (Ia) or Formula (Ib) is administered as part of a pharmaceutical composition comprising a pharmaceutical acceptable carrier, diluent, or excipient.

In some aspects, the coronavirus infection is caused by human coronavirus OC43 (HCoV-OC43), human coronavirus HKU1 (HCoV-HKU1), human coronavirus 229E (HCoV-229E), human coronavirus NL63 (HCoV-NL63), middle east respiratory syndrome-related coronavirus (MERS-CoV), severe acute respiratory syndrome coronavirus (SARS-CoV), or severe acute respiratory syndrome coronavirus 2 (SARS-CoV-2).

In one aspect, the disclosure provides a method for treating or reducing the symptoms associated with a coronavirus infection, the method comprising administering to a subject in need thereof a therapeutically effective amount of a compound of Formula (Ia) or Formula (Ib) or a composition thereof as disclosed herein.

Symptoms of the infectious disease caused by a coronavirus infection include, but are not limited to, fever, cough, and shortness of breath. Although not precluded, treating the coronavirus infection may not require that the disease or symptoms associated therewith be completely eliminated. However, in one aspect, administration of Formula (Ia) or Formula (Ib) leads to complete elimination of the coronavirus infection and associated symptoms.

In some aspects, the disclosure provides a method for treating a coronavirus infection, the method comprising administering to a subject in need thereof a therapeutically effective amount of a compound of Formula (Ia) or Formula (Ib) or a composition thereof as disclosed herein.

In some aspects, the disclosure provides a method for reducing the symptoms associated with a coronavirus infection, the method comprising administering to a subject in need thereof a therapeutically effective amount of a compound of Formula (Ia) or Formula (Ib) or a composition thereof as disclosed herein.

Compounds of Formula (Ia) or Formula (Ib) can also be administered to a mammal as part of a pharmaceutical composition containing the compound combined with a suitable pharmaceutical y acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

Pharmaceutical compositions within the scope of the present disclosure include all compositions where a compound of Formula (Ia) or Formula (Ib) is combined with one or more pharmaceutically acceptable carriers. In one aspect, the compound of Formula (Ia) or Formula (Ib) is present in the composition in an amount that is effective to achieve its intended therapeutic purpose. While individual needs may vary, a determination of optimal ranges of effective amounts of each compound is within the skill of the art. Typically, a compound of the disclosure can be administered to a mammal, e.g., a human, orally at a dose of from about 0.0025 to about 1500 mg per kg body weight of the mammal, or an equivalent amount of a pharmaceutically acceptable salt or solvate thereof, per day to treat the particular disorder. A useful oral dose of a compound of Formula (Ia) or Formula (Ib) administered to a mammal is from about 0.0025 to about 50 mg per kg body weight of the mammal, or an equivalent amount of the pharmaceutically acceptable salt or solvate thereof. For intramuscular injection, the dose is typically about one-half of the oral dose.

A unit oral dose may comprise from about 0.01 mg to about 1 g of the compound of Formula (Ia) or Formula (Ib), e.g., about 0.01 mg to about 500 mg, about 0.01 mg to about 250 mg, about 0.01 mg to about 100 mg, 0.01 mg to about 50 mg, e.g., about 0.1 mg to about 10 mg, of the compound. The unit dose can be administered one or more times daily, e.g., as one or more tablets or capsules, each containing from about 0.01 mg to about 1 g of the compound, or an equivalent amount of a pharmaceutically acceptable salt or solvate thereof.

A compound of Formula (Ia) or Formula (Ib) or pharmaceutical composition comprising a compound of Formula (Ia) or Formula (Ib) can be administered to any subject, e.g., a patient suffering from a coronavirus infection in need thereof, that may experience the beneficial effects of a compound of Formula (Ia) or Formula (Ib). Foremost among such subject are mammals, e.g., humans and companion animals, although the disclosure is not intended to be so limited. In one aspect, the subject is a human.

A pharmaceutical composition of the present disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

In one aspect, a pharmaceutical composition of the present disclosure can be administered orally. In some aspects, a pharmaceutical composition of the present disclosure can be administered orally and is formulated into tablets, dragees, capsules, or an oral liquid preparation. In one aspect, the oral formulation comprises extruded multiparticulates comprising the compound of Formula (Ia) or Formula (Ib).

Alternatively, a pharmaceutical composition of the present disclosure can be administered rectally, and is formulated in suppositories.

Alternatively, a pharmaceutical composition of the present disclosure can be administered by injection.

Alternatively, a pharmaceutical composition of the present disclosure can be administered transdermally.

Alternatively, a pharmaceutical composition of the present disclosure can be administered by inhalation or by intranasal or transmucosal administration.

Alternatively, a pharmaceutical composition of the present disclosure can be administered by the intravaginal route.

A pharmaceutical composition of the present disclosure is manufactured in a manner which itself will be known in view of the instant disclosure, for example, by means of conventional mixing, granulating, dragee-making, dissolving, extrusion, or lyophilizing processes. Thus, pharmaceutical compositions for oral use can be obtained by combining the active compound with solid excipients, optionally grinding the resulting mixture and processing the mixture of granules, after adding suitable auxiliaries, if desired or necessary, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Examples of other pharmaceutical preparations that can be used orally include push-fit capsules made of gelatin, or soft, sealed capsules made of gelatin and a plasticizer such as glycerol or sorbitol. The push-fit capsules can contain a compound in the form of granules, which can be mixed with fillers such as lactose, binders such as starches, and/or lubricants such as talc or magnesium stearate and, optionally, stabilizers, or in the form of extruded multiparticulates. In soft capsules, the active compounds are preferably dissolved or suspended in suitable liquids, such as fatty oils or liquid paraffin. In addition, stabilizers can be added.

Possible pharmaceutical preparations for rectal administration include, for example, suppositories, which consist of a combination of one or more active compounds with a suppository base. Suitable suppository bases include natural and synthetic triglycerides, and paraffin hydrocarbons, among others. It is also possible to use gelatin rectal capsules consisting of a combination of active compound with a base material such as, for example, a liquid triglyceride, polyethylene glycol, or paraffin hydrocarbon.

Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

In some aspects, the present disclosure provides kits which comprise a compound of Formula (Ia) or Formula (Ib) (or a composition comprising a compound of Formula (Ia) or Formula (Ib)) packaged in a manner that facilitates their use to practice methods of the present disclosure. In one aspect, the kit includes a compound of Formula (Ia) or Formula (Ib) (or a composition comprising a compound of Formula (Ia) or Formula (Ib)) packaged in a container, such as a sealed bottle or vessel, with a label affixed to the container or included in the kit that describes use of the compound or composition to practice the method of the disclosure. In one aspect, the compound or composition is packaged in a unit dosage form. The kit further can include a device suitable for administering the composition according to the intended route of administration.

In some aspects, the disclosure provides a method for inhibiting coronavirus pathogenicty and symptoms by administering to a subject in need thereof a therapeutically effective amount of a compound that is chemically complementary to the Mac1 channel and interactions defined and implied by the crystal structures of the Mac1/PARG-345 (MES8-4) complex and the Mac1/PARG-329 complex with their neighboring bound water molecules.

### Compounds of Formula (Ia) or Formula (Ib)

In some aspects, the disclosure provides a compound of Formula (Ia) or Formula (Ib):

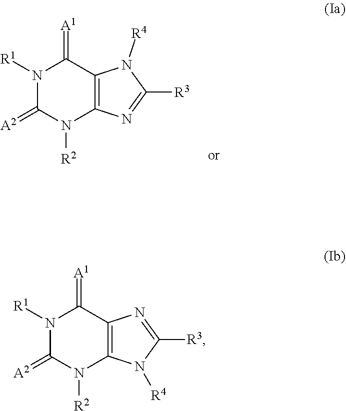

wherein:
A¹ and A² are independently O, S, or NH;
R¹ and R² are independently unsubstituted C₁-C₆ alkyl, (hydroxy)C₁-C₆ alkyl, or (C₃-C₆ cycloalkyl)C₁-C₆ alkyl; and
R³ is H or —X—Y—R⁵;
R⁴ is H or —Y′—R^{5′};
X is absent, S, O, or NH;
Y is —(CH₂)ₙ— or —(CH₂)ₘ(C═O)—;
n is an integer of 1, 2, 3, 4, 5, or 6;
m is an integer of 0, 1, 2, 3, 4, 5, or 6;
Y′ is —(CH₂)_{n′}— or —(CH₂)_{m′}(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo,

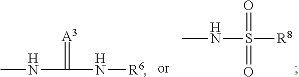

A³ is O, S, or NH;
R⁶ is optionally substituted C₁-C₆ alkyl or S(═O)₂R⁷;
R⁷ is optionally substituted C₁-C₆ alkyl, or optionally substituted (3- to 10-membered heterocyclo)C₁-C₆ alkyl;
R⁸ is optionally substituted (3- to 6-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo,

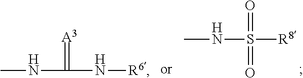

A³ is O, S, or NH;
R^{6′} is optionally substituted C₁-C₆ alkyl or S(═O)₂R⁷;
R^{7′} is optionally substituted C₁-C₆ alkyl, or optionally substituted (3- to 10-membered heterocyclo)C₁-C₆ alkyl; and
R^{8′} is optionally substituted (3- to 6-membered heterocyclo)C₁-C₆ alkyl;
or a pharmaceutically acceptable salt thereof,
   with the proviso that R³ and R⁴ are not both H.

In some aspects, the disclosure provides a compound of Formula (Ia).

In some aspects, the disclosure provides a compound of Formula (Ib).

In some aspects, the disclosure provides a compound of Formula (Ia) or Formula (Ib), wherein A¹ and A² are independently 0 or S.

In some aspects, the disclosure provides a compound of Formula (Ia) or Formula (Ib), wherein R¹ and R² are independently unsubstituted C₁-C₆ alkyl.

In some aspects, the disclosure provides a compound of Formula (Ia) or Formula (Ib), wherein R¹ and R² are methyl.

In some aspects, the disclosure provides a compound of Formula (Ia) or Formula (Ib), wherein R³ is H and R⁴ is —Y′—R^{5′}.

In some aspects, the disclosure provides a compound of Formula (Ia) or Formula (Ib), wherein:
Y′ is —(CH₂)_{n′}—,
n′ is an integer of 1 or 2;
R^{5′} is optionally substituted aryl or

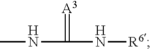

A³ is O, S, or NH;
R^{6′} is S(═O)₂R^{7′}; and
R^{7′} is optionally substituted C₁-C₆ alkyl or optionally substituted (3- to 6-membered heterocyclo)C₁-C₆ alkyl.

In some aspects, the disclosure provides a compound of Formula (Ia) or Formula (Ib), wherein:
R^{5′} is

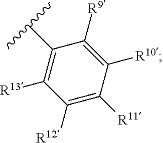

R^{9′}, R^{10′}, R^{11′}, R¹², and R¹³ are independently H, chloro, fluoro, C₁-C₆ alkyl, (CHO)C₁-C₆ alkyl,

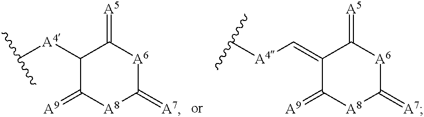

A⁴ is —(CH₂)ₚ—;
p is an integer of 1, 2, 3, 4, 5, or 6;
A⁴ is —(CH₂)_{q}—;
q is an integer of 1, 2, 3, 4, 5, or 6;
A⁴ is C₁-C₆ alkyl;
A⁵, A⁷, and A⁹ are independently O, S, or NH; and
A⁶ and A⁸ are independently NH or CH₂.

In some aspects, the disclosure provides a compound of Formula (Ia) or Formula (Ib), wherein:
R^{5′} is

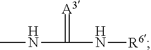

A^{3′} is O;
R^{6′} is S(═O)₂R^{7′}; and
R^{7′} is C₁-C₆ alkyl.

In some aspects, the disclosure provides a compound of Formula (Ia) or Formula (Ib), wherein R³ is —X—Y—R⁵ and R⁴ is H.

In some aspects, the disclosure provides a compound of Formula (Ia) or Formula (Ib), wherein:
X is S or NH;
Y is —(CH₂)ₙ— or —(CH₂)ₘ(C═O)—;
n is an integer of 1 or 2;
m is an integer of 0, 1, or 2;
R⁵ is optionally substituted 3- to 10-membered heterocyclo or

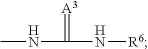

A³ is O, S, or NH; and
R⁶ is optionally substituted 3- to 6-membered (heterocyclo)C₁-C₆ alkyl.

In some aspects, the disclosure provides a compound of Formula (Ia) or Formula (Ib), wherein:
R⁵ is

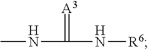

A³ is S; and
R⁶ is (morpholine)C₁-C₆alkyl.

In some aspects, the disclosure provides a compound of Formula (Ia) or Formula (Ib), wherein R⁵ is selected from the group consisting of:

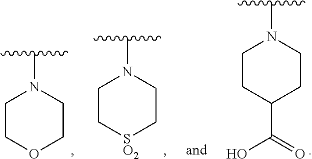

In some aspects, the disclosure provides a compound of Formula (Ia) or Formula (Ib), wherein:
Y is —(CH₂)ₘ(C═O)—;
m is 2;
R⁵ is:

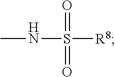

and
R⁸ is optionally substituted (6-membered heterocyclo)C₁-C₃ alkyl.

In some aspects, the disclosure provides a compound of Formula (Ia) or Formula (Ib wherein R⁸ is:

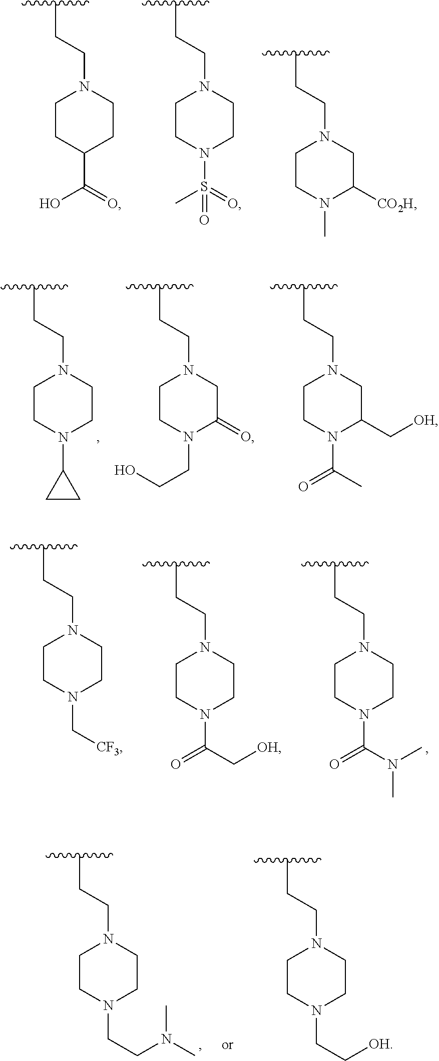

In some aspects, the disclosure provides a compound of Formula (Ia), wherein the compound is selected from the group consisting of:

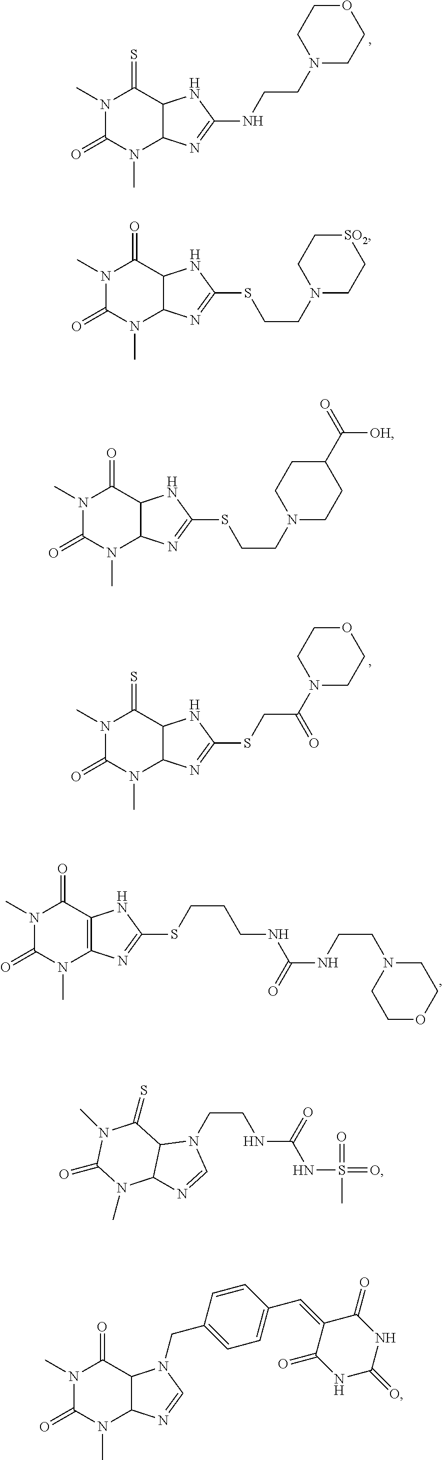

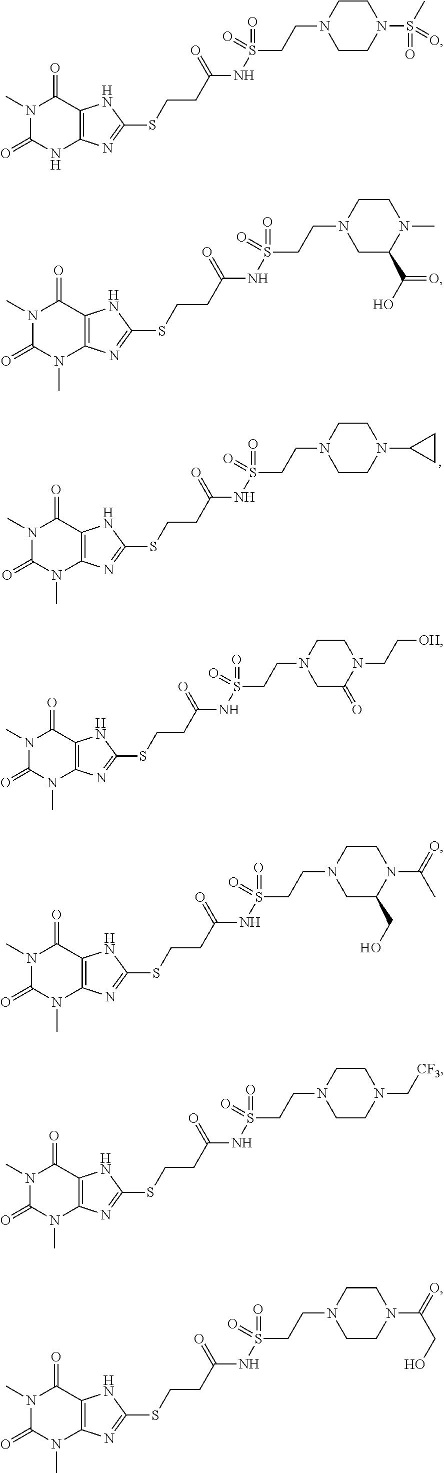

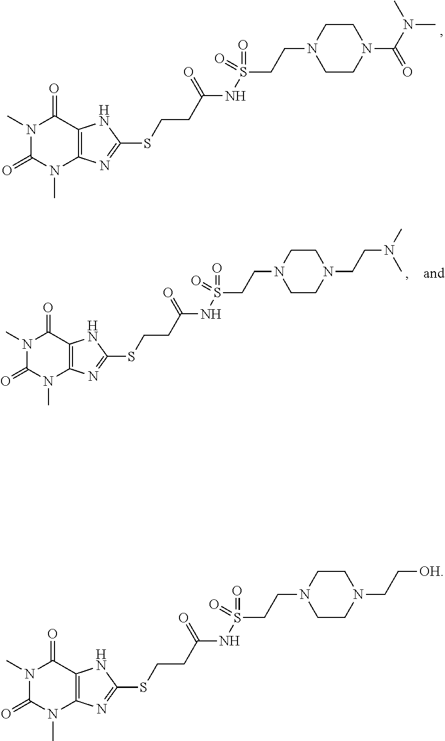 [SMILES: CN(C)C(=O)N1CCN(CCS(=O)(=O)NC(=O)CCSc2nc3c([nH]2)c(=O)n(C)c(=O)n3C)CC1 ; CN(C)CCN1CCN(CCS(=O)(=O)NC(=O)CCSc2nc3c([nH]2)c(=O)n(C)c(=O)n3C)CC1 ; Cn1c(=O)c2[nH]c(SCCC(=O)NS(=O)(=O)CCN3CCN(CCO)CC3)nc2n(C)c1=O]

In some aspects, the disclosure provides a compound of Formula (Ib), wherein the compound is selected from the group consisting of:

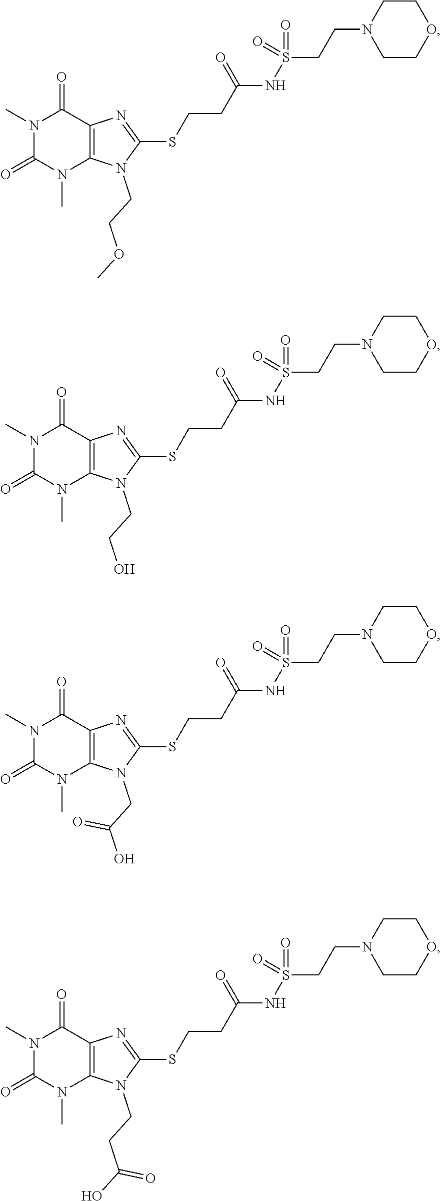 [SMILES: COCCn1c(SCCC(=O)NS(=O)(=O)CCN2CCOCC2)nc2c(=O)n(C)c(=O)n(C)c21 ; Cn1c(=O)c2nc(SCCC(=O)NS(=O)(=O)CCN3CCOCC3)n(CCO)c2n(C)c1=O ; Cn1c(=O)c2nc(SCCC(=O)NS(=O)(=O)CCN3CCOCC3)n(CC(=O)O)c2n(C)c1=O ; Cn1c(=O)c2nc(SCCC(=O)NS(=O)(=O)CCN3CCOCC3)n(CCC(=O)O)c2n(C)c1=O]

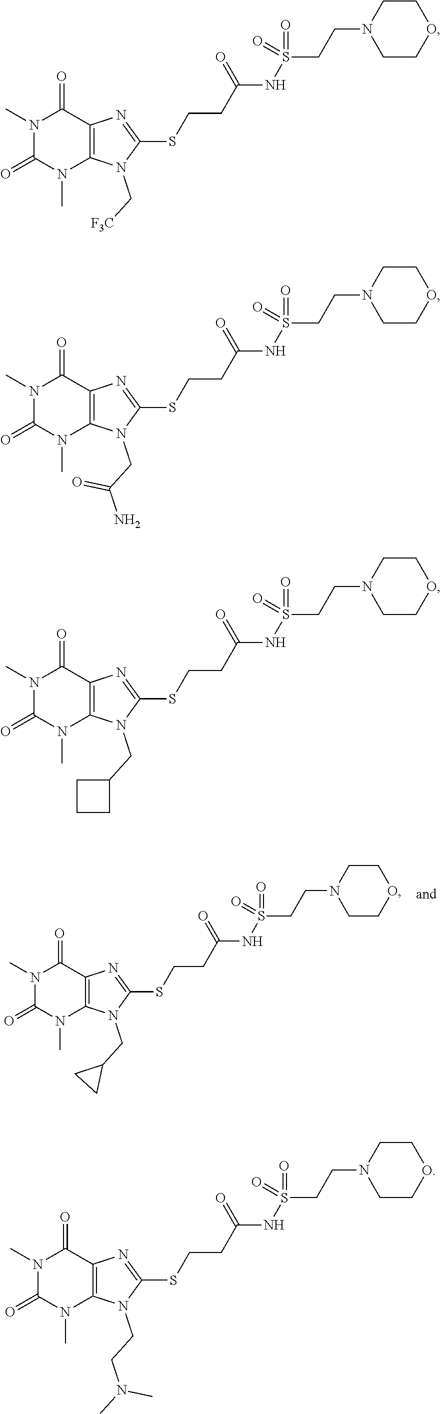 [SMILES: Cn1c(=O)c2nc(SCCC(=O)NS(=O)(=O)CCN3CCOCC3)n(CC(F)(F)F)c2n(C)c1=O ; Cn1c(=O)c2nc(SCCC(=O)NS(=O)(=O)CCN3CCOCC3)n(CC(N)=O)c2n(C)c1=O ; Cn1c(=O)c2nc(SCCC(=O)NS(=O)(=O)CCN3CCOCC3)n(CC3CCC3)c2n(C)c1=O ; Cn1c(=O)c2nc(SCCC(=O)NS(=O)(=O)CCN3CCOCC3)n(CC3CC3)c2n(C)c1=O ; CN(C)CCn1c(SCCC(=O)NS(=O)(=O)CCN2CCOCC2)nc2c(=O)n(C)c(=O)n(C)c21]

In some aspects, the disclosure provides a pharmaceutical composition comprising a compound of Formula (Ia) or Formula (Ib) and a pharmaceutically acceptable carrier, diluent, or excipient.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

The compounds of the present disclosure may be prepared in a number of ways well known to one skilled in the art of organic synthesis. More specifically, the novel compounds of this disclosure may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed, Such restrictions to the substituents, which are not compatible with the reaction conditions, will be apparent to one skilled in the art and alternate methods must then be used. Unless otherwise stated, the starting materials for the examples contained herein are either commercially available or are readily prepared by standard methods from known materials. The compounds of Formula (Ia) or Formula (Ib) may be synthesized through standard organic chemistry methodology and purification known to those trained in the art of organic synthesis by using commercially available starting materials and reagents.

### Example 1: Phylogenic Analysis

### Methods and Materials

### Identification of the MacroD2 and Gdap2 Genes in the Extremophile Alvinella Pompejana

The de novo assembler SoapDeNovo2 was used on a library of short reads generated from A. pompejana samples collected during past expeditions (2003-2004) in the East Pacific Rise (Shin et al., 2009) to obtain a partial assembly of the annelid genome. To the scaffolds obtained a curated set of ESTs (Holder et al., 2013) was added, from which we selected a training set of full-length transcripts to conduct a gene prediction analysis using AUGUSTUS (Stanke and Morgenstern, 2005). The human UNIPROT entries A1Z1Q3 for MACROD2 and Q9NXN4 for GDAP2 were then used to query the augustus.gtf prediction dataset using blastp. Bedtools (version 2.25.0) were then used to extract the nucleotide sequences from the scaffolds and the amino acid sequences from the augustus.aa prediction dataset. The top blastp scores were 3e-85 for MACROD2 and 9e-168 for GDAP2. For GDAP2 we found support from both the genome assembly and from a full-length EST. The A. pompejana protein sequences were further used in a blastp search against the Protostomia clade to verify that top homology was indeed with the same human gene bait. A. pompejana MacroD2 and GDAP2 amino acid sequences are reported in Table 1-1.

TABLE 1-1
Alvi pompejana MacroD2 and GDAP2 Sequences
A. pompejana MacroD2 (gp30765.t1)

| | |
|---|---|
| MLTKRILAGRRLMSANRHRPIPDRICSLRRQ | SEQ ID NO: 1 |
| SRATFSSQSDRGSDSLIPRVFRTIVGWFAAH | |
| LPRRGEGNGLGLDEDEKDALNVNSQHAKKKQ | |
| KWQIEKEEYLKMPISEKRKIYKTQYVTLDSV | |
| PTWPEYYKKNLEASKVKPSKEPVDEALNSKV | |
| SLWCGDITTLEIDCIVNAANESLLGGGGVDG | |
| AIHRAAGPTLLAECRTACGCKTGDAKITGGY | |
| RLPAKYIIHTVGPRYHGDEKLRSCYKKSLDL | |
| MIENNLHSIAFPCISTGIYGFPGERAADIAL | |
| TTVKDFLQKHKDKVDRIIFCLFTRDDVNIYE | |
| SKMPTYFPVEGQCSDHEDQTTDDNPNVDLPG | |
| KPSDKNSVPKL | |

A. pompejana GDAP2 (g72977.t1)

| | |
|---|---|
| MDDPLAAPTQATVDHTKLVRWNQTNVPEYNM | SEQ ID NO: 2 |
| VIDNPDDKKKSPFAYNREINQKIVLWSGDIT | |
| ALDTEAILHSTNETLSDVYPASERLLKRAGP | |
| DLQKDLSSNVKVCRTGEARLTKGYQLPARYV | |
| IHTVGPRYNLKYKTAAESALFNSYRSVLQIV | |
| REKQMSSVALCCIHASRRGYPPQEGAHIALR | |
| TVRRFLEKYGDTIDTVVFVVTGEDEDVYISL | |
| LPLYFPRSEHEEDFAAYQLPDDVGNEDGEPV | |
| IKERQIRIMGKPAYEAQKFNWKSPEELEESI | |
| NINEAFDTSIAVGAHSFSKMDDDIDKKRRIR | |
| LQYETHIALLNKEQYKRYEKWLKRSYQEDLS | |
| PMESLRCLYQSGFDVYGRPVVVFIGRHFPAT | |
| KIDLDKFTLYLVQLMDNIVNKPYVIVYFHTL | |
| TQSDNHLDAGYLRSLYNLLDSRYKQNLGAVY | |
| VVHPTFWSKVMTWFFMTFNTTDLKSRIHNIP | |
| GLEYLFKRIPMDQLDIPDFISDYDIQVHGTR | |
| YYNPDVDKNL | |

### Macrodomain Phylogeny Analysis

Macrodomain sequences were selected to represent the six macrodomain subfamilies and were partially based on the listing described in (Rack et al., 2016). Macrodomain sequences were extracted from each gene ID (Table 1-2) and aligned using the structure- and sequence-informed Espresso pipeline of the T-Coffee multiple sequence alignment web server (Armougom et al., 2006; Notredame et al., 2000). A phylogenetic tree was calculated from the resulting sequence alignment as follows. The evolutionary history was inferred using the Neighbor-Joining method (Saitou and Nei, 1987). The bootstrap consensus tree inferred from 500 replicates is taken to represent the evolutionary history of the taxa analyzed (Felsenstein, 1985). Branches corresponding to partitions reproduced in less than 50% bootstrap replicates are collapsed. The evolutionary distances were computed using the p-distance method (Nei, 2000) and are in the units of the number of amino acid differences per site. This analysis involved 48 amino acid sequences. All ambiguous positions were removed for each sequence pair (pairwise deletion option). There were a total of 7365 positions in the final dataset. Phylogenetic analyses were conducted in MEGA X (Kumar et al., 2018; Stecher et al., 2020).

TABLE 1-2
Reference List of Curated Macrodomain Sequences with Gene IDs and Figure Abbreviations
MacroD
WP_010922344.1 protein-ADP-ribose hydrolase [Streptococcus pyogenes]
WP_000449060.1 protein-ADP-ribose hydrolase [Staphylococcus aureus]
NP_564960.1 appr-1-p processing enzyme family protein 85-223 [Arabidopsis thaliana]
NP_060156.1 ganglioside-induced differentiation-associated protein 2 isoform a 54-193 [Homo
sapiens]
NP_001004563.1 ganglioside-induced differentiation-associated protein 2 65-204 [Danio rerio]
g72977.t1 ganglioside-induced differentiation-associated protein 2 (predicted) 52-22
NP_001004573.2 ADP-ribose glycohydrolase MACROD1 148-314 [Danio rerio]
g30765.t1 ADP-ribose glycohydrolase MACROD2 (predicted) 154-314
XP_005273996.1 ADP-ribose glycohydrolase MACROD1 isoform X1 153-319 [Homo sapiens]
NP_542407.2 ADP-ribose glycohydrolase MACROD2 isoform 1 71-237 [Homo sapiens]
NP_956843.1 ADP-ribose glycohydrolase MACROD2 62-228 [Danio rerio]
WP_010916977.1 O-acetyl-ADP-ribose deacetylase [Thermoplasma volcanium]
NP_030605.2 appr-1-p processing enzyme family protein 82-246 [Arabidopsis thaliana]
VWQ01961.1 ymdB [Escherichia coli]
WP_013910118.1 macro domain-containing protein [Thermodesulfobacterium geofontis]
WP_010888916.1 macro domain-containing protein [Deinococcus radiodurans]
XP_005247877.1 protein mono-ADP-ribosyltransferase PARP9 isoform X1 117-295 [Homo sapiens]
NP_060024.2 protein mono-ADP-ribosyltransferase PARP14 801-977 [Homo sapiens]
XP_017213521.2 poly [ADP-ribose] polymerase 14 isoform X1 785-962 [Danio rerio]
YP_009742610.1 Non-structural protein 3 MacroX 1054-11779 [SARS coronavirus 2 (2697049)]
AHY61336 ORF1ab Nsp3 macrodomain 1243-1344 (pred) [BtVs-BetaCoV/SC2013]
YP_009047215.1 Nsp3 macrodomain 290-390 (pred) [Middle East respiratory syndrome-related
coronavirus]
MacroH2A
NP_061119.1 core histone macro-H2A.2 178-364 [Homo sapiens]
NP_001020673.1 core histone macro-H2A.2 173-359 [Danio rerio]
NP_613258.2 core histone macro-H2A.1 isoform 3 178-364 [Homo sapiens]
NP_001035451.1 core histone macro-H2A.1 170-349 [Danio rerio]
XP_004347529.1 histone macroH2A1.1 178-360 [Capsaspora owczarzaki ATCC 30864]
XP_005247877.1 protein mono-ADP-ribosyltransferase PARP9 isoform X1 317-453 [Homo sapiens]
NP_060024.2 protein mono-ADP-ribosyltransferase PARP14 1014-1155 [Homo sapiens]
NP_060024.2 protein mono-ADP-ribosyltransferase PARP14 1227-1354 [Homo sapiens]
NP_001106995.1 protein mono-ADP-ribosyltransferase PARP15 isoform 1 89-230 [Homo sapiens]
NP_001106995.1 protein mono-ADP-ribosyltransferase PARP15 isoform 1 304-431 [Homo sapiens]
XP_017213521.2 poly [ADP-ribose] polymerase 14 isoform X1 1059-1151 [Danio rerio]
XP_017213521.2 poly [ADP-ribose] polymerase 14 isoform X1 1264-1396 [Danio rerio]
ALC1
NP_004275.4 chromodomain-helicase-DNA-binding protein 1-like isoform 1 716-866 [Homo sapiens]
XP_012035145.3 chromodomain-helicase-DNA-binding protein 1-like isoform X1 716-866 [Ovis
aries]
NP_080815.1 chromodomain-helicase-DNA-binding protein 1-like 721-871 [Mus musculus]
YP_007004858.1 hypothetical protein F412 gp158 [Escherichia phage wV7]
NP_659500.1 ADP-ribose glycohydrolase OARD1 isoform a [Homo sapiens]
NP_001344833.1 ADP-ribose glycohydrolase OARD1 [Mus musculus]
NP_001018591.1 ADP-ribose glycohydrolase OARD1 [Danio rerio]
WP_003400551.1 MULTISPECIES macro domain-containing protein 1-139 [Mycobacterium
tuberculosis complex]
PARG
WP_041439432.1 TIGR02452 family protein [Thermomonospora curvata]
XP_001748857.1 uncharacterized protein MONBRDRAFT_33864 91-370 [Monosiga brevicollis
MX1]
XP_642024.1 poly glycohydrolase 265-658 [Dictyostelium discoideum AX4]
NP_001077989.1 Poly (ADP-ribose) glycohydrolase 1 83-547 (PARG) [Arabidopsis thaliana]
NP_850175.1 poly(ADP-ribose) glycohydrolase 2 85-522 [Arabidopsis thaliana]
XP_687541.4 poly(ADP-ribose) glycohydrolase 376-777 [Danio rerio]
NP_003622.2 poly(ADP-ribose) glycohydrolase isoform a 581-976 [Homo sapiens]
Macro2
NP_013805.1 putative ADP-ribose 1″-phosphate phosphatase [Saccharomyces cerevisiae S288C]
YP_001949930.1 unnamed protein product [Ralstonia phage phiRSL1]
XP_003388308.2 PREDICTED: uncharacterized protein LOC100640713 isoform X1 [Amphimedon
queenslandica]
SUD-M
AHY61336 ORF1ab Nsp3 SUD-M 1403-1484 (pred) [BtVs-BetaCoV/SC2013]
YP_009047215.1 Nsp3 SUD-M 428-531 (pred) [Middle East respiratory syndrome-related
coronavirus]
YP_009742610.1|Non-structural protein 3 SUD-N 415-540 [SARS coronavirus 2 (694009)]
YP_009742610.1|Non-structural protein 3 SUD-M 533-675 [SARS coronavirus 2 (694009)]

### Evolutionary Trace Analysis

Protein-specific evolutionary traces were constructed for CoV-2 Nsp3 (sequence obtained from the NC_045512.2 genome) and human PARG (NP_003622 sequence) as described in (Mihalek et al., 2004). The homologous sequences for each trace were obtained by BLAST using blastall 2.2.15 (Altschul et al., 1997) and the NCBI nr, UniRef90, and Uniref100 databases (Pruitt et al., 2007; Suzek et al., 2015). The homologous sequences were automatically selected to represent different phylogenetic distances to the sequence of interest while minimizing alignment gaps. The sequences were aligned using MUSCLE (Edgar, 2004). The resulting Evolutionary Trace ranks were mapped onto the PDB structures: 6W02 (CoV-2 Mac1) and 41BG (human PARG), respectively, using the PyETV plugin (Lua and Lichtarge, 2010). Macrodomain-specific evolutionary traces were constructed for the greater macrodomain family using the structure-informed multiple sequence alignment (MSA) from curated macrodomain subfamilies (Table 1-2). ET values from this broader MSA comparison were mapped to CoV-2 Mac1 and PARG structures, assuming each respectively as the reference sequence for the ET analysis.

### Results and Discussion

### Phylogenetic Analysis Assigns CoV-2 Mac1 to the MacroD Subfamily

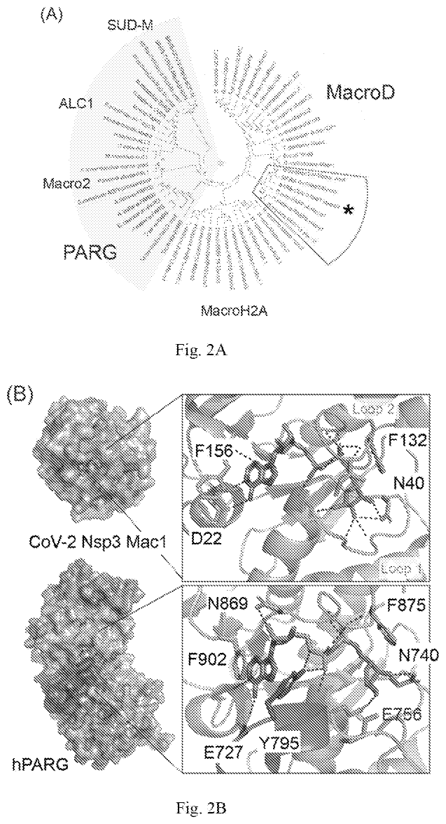

Prior to applying the PARGi library to CoV-2 Mac1 inhibitor development, phylogenetic and evolutionary analyses were created of these two systems to evaluate the similarity of their active sites. To this end, a phylogenetic tree of the macrodomain family was constructed, based upon a sequence- and structure-informed alignment of curated eukaryotic, bacterial, and viral macrodomain sequences from the six macrodomain subfamilies (FIG. 2A, Table 1-1, and Table 1-2). Consistent with their mono(ADP-ribosyl) hydrolase activity, the beta coronavirus Mac1 domains are most closely related to the MacroD subfamily. Notably, the closest human homologues to these viral macrodomains are the N-terminal macrodomains (Mac1) of PARP9 and PARP14. PARP9 and PARP14 activities are implicated in regulation of macrophage activation (Iwata et al., 2016) and promotion of interferon responses in cells (Caprara et al., 2018; Zhang et al., 2015).

The PARG macrodomain subfamily associates to a separate phylogenetic branch relative to the MacroD subfamily and CoV-2 Mac1. In addition to the macrodomain core, PARG enzymes feature an N-terminal accessory domain and small C-terminal helical extension. Mammalian PARG macrodomains contain a unique β-hairpin insert which forms a ‘tyrosine clasp’ (Y795 in humans) at the ADP-ribose active site that makes contacts with the adenine ring and O5′ and O1A of the α-phosphate (Kim et al., 2012) (FIG. 2B). Without being bound by theory, these structural distinctions are presumed to support PARG's ability to recognize and hydrolyze the O-glycosidic bond of poly(ADP-ribose) substrates.

### Example 2: Virtual Screening

### Methods and Materials

### Virtual Screening of the PARGi Library Against COV-2 Mac1

The CoV-2 Mac1 crystal structure with MES exhibits a more open active site relative to the published unliganded (PDB: 6WEY) and (ADP-ribose)-bound (PDB: 6W02) structures and was thus selected as a target for virtual screening. Specifically, the CoV-2 Mac-1 MES structure rotates the F156 phenyl side chain upright to make a π-π stacking interaction with the adenosyl pocket and includes a larger opening between Loops 1 and 2. Waters and the MES ligand were removed, and the structure was minimized using Schrödinger's Protein Preparation wizard (Schrödinger Suite 2019-2 with Epik, Impact, and Prime, Schrödinger, LLC, New York, NY, 2019). The docking grid was focused on the center of the ADP-ribose pocket (Pro125) with a 15 Å-cubic box, excluding ligands longer than 20 Å.

Structure data files (SDF) for the 300-compound library of JA2131 PARGi derivatives were extracted from SMILES strings and prepared for docking in Maestro with LigPrep (Schrödinger Suite 2019-2). The SDF for the control ADP-ribose ligand was retrieved from PubChem (https://pubchem.ncbi.nlm.nih.gov) and also prepared with LigPrep. Virtual high-throughput screening of the PARGi library against the CoV-2 Mac1 target was carried out in Maestro with flexible ligand docking in both standard (SP) and extra-precision modes (XP). MM-GBSA binding energies (AG) were calculated for each pose and used to rank the docking results. Poses from the XP screening returned highly variable and unrealistically compacted ligand geometries, suggesting an artifact from the more stringent scoring function. In contrast, MM-GBSA ranked poses from the SP screening returned more consistent and realistic ligand conformations. Thus, the top 20 MM-GBSA ranked poses from the SP screening protocol were selected for further analysis. This approach was repeated with rationally designed Mac1 inhibitors to generate poses for CoV-2 Mac1 binding and to rank these compounds against the PARGi library. Docking poses were visualized and analyzed with PyMOL (The PyMOL Molecular Graphics System, Version 2.0 Schrödinger, LLC) and Chimera (Pettersen et al., 2004). Computational suites were accessed through the SBGrid Consortium (Morin et al., 2013) Virtual screening identifies candidate PARGi fragments for engaging COV-2 Mac1.

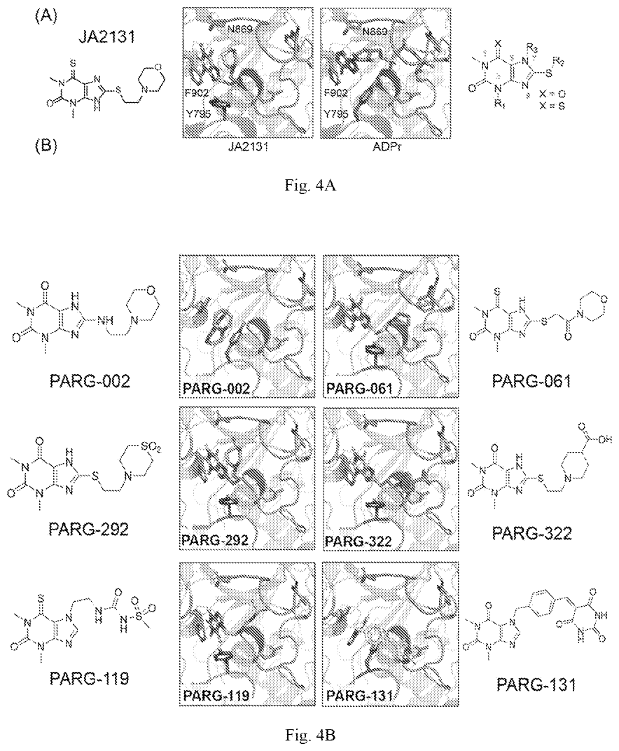

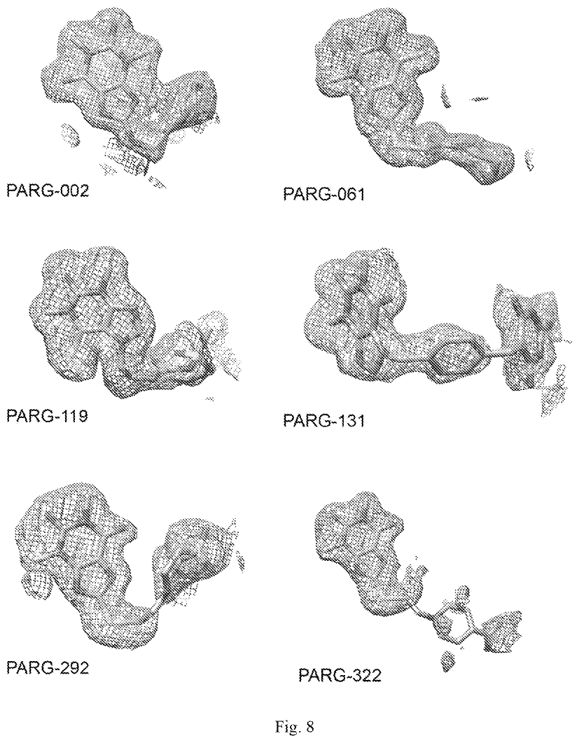

A JA2131 derivative library of over 300 compounds as part of a PARGi optimization pipeline was generated, incorporating a variety of substitutions at the N3, N7, and C8 positions of the methylxanthine scaffold (FIG. 4A and FIG. 4B). A focused set of compounds were tested by determining their X-ray crystal structures bound to PARG (Table 2-1 and FIG. 8). While JA2131 anchors compactly into the adenosyl pocket of the PARG active site, aromatically engaging F902 and Y795 (FIG. 4A), other members of the derivative library exhibit more varied and elongated conformations that would be more adaptable to the extended active site conformations observed for ADP-ribose (FIG. 4B and FIG. 8). The absence of a tyrosine clasp in CoV-2 Mac1 is also expected to increase conformational accessibility to the active site.

TABLE 2-1
Crystallographic Statistics for PARG/Inhibitor Complexes

| | | | CoV-2 Mac1/ | | CoV-2 Mac1/ | | | | | |
|---|---|---|---|---|---|---|---|---|---|---|
| | CoV-2 Mac1 | | PARG-329 | | PARG-345 | | PARG-002 | | PARG-061 | |
| Wavelength | 0.9795 | | 1.000 | | 0.9795 | | 0.9795 | | 0.9795 | |
| Resolution range | 38.11-1.45 | | 36.55-1.55 | | 38.34-2.3 | | 38.66-1.65 | | 38.33-1.43 | |
| | (1.502-1.45) | | (1.605-1.55) | | (2.382-2.3) | | (1.709-1.65) | | (1.481-1.43) | |
| Space group | P 31 2 1 | | P 31 2 1 | | P 31 2 1 | | P 21 21 21 | | P 21 21 21 | |
| Unit cell | 112.594 112.594 | | 113.636 113.636 | | 113.097 113.097 | | 66.463 88.964 | | 66.408 89.201 | |
| | 41.407 90 90 120 | | 39.37 90 90 120 | | 41.659 90 90 120 | | 95.049 90 90 90 | | 93.877 90 90 90 | |
| Total reflections | 544340 | (53755) | 408391 | (38298) | 138127 | (13265) | 892613 | (90988) | 807083 | (82401) |
| Unique reflections | 53126 | (5190) | 42536 | (4229) | 13824 | (1335) | 68294 | (6747) | 103198 | (10175) |
| Multiplicity | 10.2 | (10.4) | 9.6 | (9.1) | 10.0 | (9.9) | 13.1 | (13.5) | 7.8 | (8.1) |
| Completeness (%) | 94.14 | (80.03) | 95.98 | (88.29) | 91.72 | (76.70) | 99.74 | (99.69) | 99.58 | (98.92) |
| Mean I/sigma(I) | 11.53 | (1.35) | 10.39 | (0.95) | 13.49 | (1.05) | 24.76 | (1.74) | 13.49 | (0.81) |
| Wilson B-factor | 21.55 | | 19.6 | | 45.84 | | 26.99 | | 22.04 | |
| R-merge | 0.09795 | (3.211) | 0.1292 | (3.305) | 0.1371 | (2.482) | 0.05614 | (1.655) | 0.06384 | (2.646) |
| R-meas | 0.1032 | (3.374) | 0.1367 | (3.506) | 0.1445 | (2.616) | 0.05845 | (1.719) | 0.06836 | (2.826) |
| R-pim | 0.03222 | (1.027) | 0.04408 | (1.156) | 0.04533 | (0.819) | 0.01608 | (0.4605) | 0.02406 | (0.9804) |
| CC½ | 0.998 | (0.412) | 0.999 | (0.394) | 0.998 | (0.397) | 1 | (0.745) | 0.999 | (0.393) |
| CC* | 0.999 | (0.764) | 1 | (0.752) | 1 | (0.754) | 1 | (0.924) | 1 | (0.751) |
| Reflections used | 50444 | (4265) | 40852 | (3739) | 12690 | (1027) | 68275 | (6743) | 103043 | (10103) |
| in refinement | | | | | | | | | | |
| Reflections used | 1888 | (159) | 1907 | (177) | 630 | (51) | 3407 | (349) | 5155 | (466) |
| for R-free | | | | | | | | | | |
| R-work | 0.1573 | (0.2986) | 0.1606 | (0.3619) | 0.1801 | (0.3411) | 0.1741 | (0.2622) | 0.1724 | (0.3137) |
| R-free | 0.1774 | (0.2712) | 0.1877 | (0.4455) | 0.2060 | (0.4536) | 0.1981 | (0.3002) | 0.1919 | (0.3222) |
| CC(work) | 0.974 | (0.758) | 0.976 | (0.743) | 0.966 | (0.683) | 0.959 | (0.881) | 0.961 | (0.694) |
| CC(free) | 0.967 | (0.782) | 0.970 | (0.673) | 0.976 | (0.422) | 0.949 | (0.777) | 0.948 | (0.698) |

| | | | | | |
|---|---|---|---|---|---|
| Number of non- | 1493 | 1514 | 1424 | 4299 | 4460 |
| hydrogen atoms | | | | | |
| macromolecules | 1280 | 1284 | 1324 | 3916 | 3972 |
| ligands | 47 | 48 | 50 | 80 | 54 |
| solvent | 166 | 182 | 50 | 303 | 434 |
| Protein residues | 166 | 165 | 167 | 501 | 509 |
| RMS(bonds) | 0.014 | 0.014 | 0.009 | 0.014 | 0.005 |
| RMS(angles) | 1.42 | 1.19 | 0.81 | 1.11 | 0.87 |
| Ramachandran | 98.78 | 97.55 | 98.79 | 97.57 | 97.55 |
| favored (%) | | | | | |
| Ramachandran | 1.22 | 2.45 | 1.21 | 2.23 | 2.45 |
| allowed (%) | | | | | |
| Ramachandran | 0 | 0 | 0 | 0.2 | 0 |
| outliers (%) | | | | | |
| Rotamer outliers | 0 | 2.07 | 1.33 | 0.25 | 0 |
| (%) | | | | | |
| Clashscore | 3.05 | 3.38 | 2.18 | 1.03 | 1.52 |
| Average B-factor | 31.89 | 35.82 | 59.52 | 35.24 | 28.88 |
| macromolecules | 29.08 | 32.23 | 58.06 | 34.51 | 27.42 |
| ligands | 64.76 | 81.61 | 98.88 | 50.88 | 43.93 |
| solvent | 44.25 | 47.75 | 58.78 | 40.5 | 40.42 |
| Number of TLS | 1 | 6 | 7 | 5 | 1 |
| groups | | | | | |

Statistics for the highest-resolution shell are shown in parentheses.

TABLE 2-1
Crystallographic Statistics for PARG/Inhibitor Complexes

| | PARG-119 | | PARG-131 | | PARG-292 | | PARG-322 | |
|---|---|---|---|---|---|---|---|---|
| Wavelength | 0.9201 | | 0.9795 | | 0.9792 | | 0.9793 | |
| Resolution range | 29.58-1.9 | | 36.92-1.66 | | 64.9-1.85 | | 29.15-1.96 | |
| | (1.968-1.9) | | (1.719-1.66) | | (1.916-1.85) | | (2.03-1.96) | |
| Space group | P 21 21 21 | | P 1 21 1 | | P 21 21 21 | | P 21 21 21 | |
| Unit cell | 66.532 88.66 | | 45.293 66.579 | | 66.607 89.038 | | 66.585 88.965 | |
| | 94.426 90 90 90 | | 89.142 90 95.5 90 | | 94.799 90 90 90 | | 94.727 90 90 90 | |
| Total reflections | 264741 | (26338) | 253791 | (20803) | 507222 | (49055) | 451008 | (46056) |
| Unique reflections | 44680 | (4419) | 59073 | (4954) | 48514 | (4759) | 41080 | (4054) |
| Multiplicity | 5.9 | (6.0) | 4.3 | (4.2) | 10.5 | (10.3) | 11.0 | (11.4) |
| Completeness (%) | 99.90 | (99.98) | 94.30 | (79.57) | 99.33 | (99.31) | 99.91 | (100.00) |
| Mean I/sigma(I) | 10.52 | (2.25) | 7.85 | (1.55) | 14.26 | (1.83) | 14.94 | (3.89) |
| Wilson B-factor | 31.11 | | 23.81 | | 30.46 | | 27.28 | |
| R-merge | 0.08665 | (0.6689) | 0.09772 | (1.141) | 0.09191 | (1.356) | 0.1033 | (0.692) |
| R-meas | 0.09518 | (0.7339) | 0.1119 | (1.306) | 0.09667 | (1.428) | 0.1084 | (0.7243) |
| R-pim | 0.03888 | (0.2983) | 0.05325 | (0.6203) | 0.02945 | (0.4401) | 0.03248 | (0.2121) |
| CC½ | 0.997 | (0.76) | 0.994 | (0.59) | 0.999 | (0.697) | 0.998 | (0.902) |
| CC* | 0.999 | (0.929) | 0.999 | (0.861) | 1 | (0.906) | 1 | (0.974) |
| Reflections used in | 44676 | (4418) | 58856 | (4941) | 48495 | (4754) | 41066 | (4054) |
| refinement | | | | | | | | |
| Reflections used | 2262 | (221) | 2943 | (227) | 2316 | (227) | 2049 | (196) |
| for R-free | | | | | | | | |
| R-work | 0.1746 | (0.2311) | 0.1854 | (0.2973) | 0.1776 | (0.2924) | 0.1725 | (0.1881) |
| R-free | 0.1957 | (0.2466) | 0.2201 | (0.3299) | 0.1947 | (0.3345) | 0.2049 | (0.2492) |
| CC(work) | 0.955 | (0.872) | 0.953 | (0.845) | 0.956 | (0.860) | 0.954 | (0.929) |
| CC(free) | 0.951 | (0.859) | 0.938 | (0.771) | 0.960 | (0.843) | 0.942 | (0.886) |

| | | | | |
|---|---|---|---|---|
| Number of non- | 4139 | 4438 | 4222 | 4186 |
| hydrogen atoms | | | | |
| macromolecules | 3826 | 3952 | 3869 | 3860 |
| ligands | 58 | 54 | 65 | 25 |
| solvent | 255 | 432 | 288 | 301 |
| Protein residues | 499 | 499 | 503 | 499 |
| RMS(bonds) | 0.005 | 0.007 | 0.005 | 0.005 |
| RMS(angles) | 0.94 | 0.85 | 0.72 | 0.81 |
| Ramachandran | 97.75 | 97.94 | 97.56 | 97.54 |
| favored (%) | | | | |
| Ramachandran | 2.05 | 1.85 | 2.44 | 2.46 |
| allowed (%) | | | | |
| Ramachandran | 0.2 | 0.21 | 0 | 0 |
| outliers (%) | | | | |
| Rotamer outliers | 0 | 0.25 | 0.51 | 0.26 |
| (%) | | | | |
| Clashscore | 1.33 | 1.53 | 1.05 | 1.85 |
| Average B-factor | 34.64 | 29.54 | 36.36 | 31.02 |
| macromolecules | 34.16 | 28.32 | 35.64 | 30.49 |
| ligands | 45.71 | 50.37 | 55.78 | 41.42 |
| solvent | 39.32 | 38.11 | 41.65 | 36.86 |
| Number of TLS | 8 | 6 | 5 | 7 |
| groups | | | | |

Statistics for the highest-resolution shell are shown in parentheses.

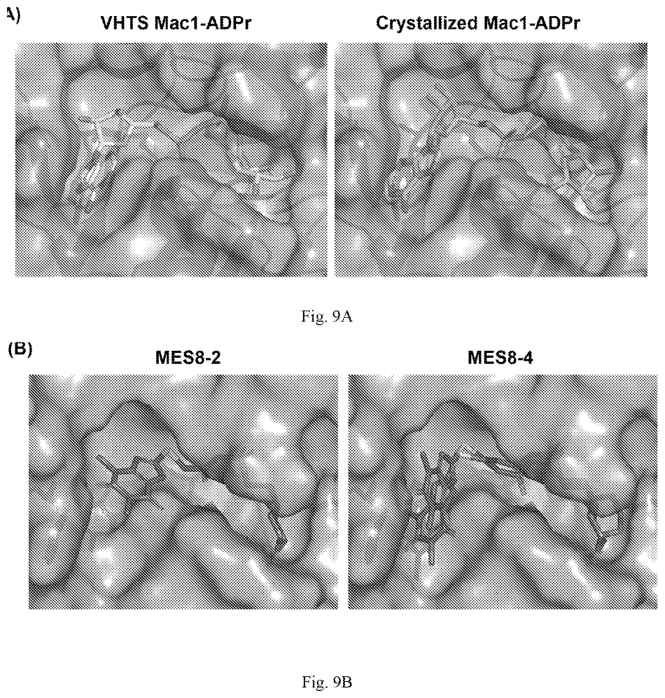

To identify candidate PARGi capable of engaging the CoV-2 Mac1 active site, an in silico screen was performed using the Maestro suite of Schrodinger to prepare and dock our PARGi ligand library. CoV-2 Mac1 crystal structures were selected as a screening target, as it presented a more open, accessible active site conformation compared to crystal structures of unliganded and (ADP-ribose)-bound domains. Binding energies (AG) were calculated for the top docked poses using the MM-GBSA method, and these energies were used to rank and extract the top twenty docked complexes (Table 2-2). As a control, ADP-ribose was included in the docking and was successfully captured in the MM-GBSA ranking strategy. The top-scoring Mac1-ADP-ribose complex effectively captures the ligand binding orientation of the crystal structure (FIG. 9A), placing the adenosyl and distal ribose groups in their respective pockets and aligning the pyrophosphate linker with Loops 1 and 2. The selected APDr poses exhibit rotational variability among the adenosyl and distal ribose groups, but maintain the overall ligand binding path.

TABLE 2-2
MM/GBSA-Ranked PARGi from CoV-2 Nps3 Mac1 Virtual Screening

| | MMGBSA | | |
|---|---|---|---|
| | ΔG | | Anchor |
| PARGi | (kcal/mol) | Chemotype | Point |
| ADPr | −74 | 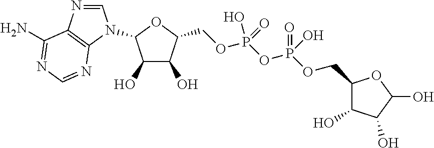 [SMILES: Nc1ncnc2c1ncn2[C@@H]1O[C@H](COP(=O)(O)OP(=O)(O)OC[C@H]2OC(O)[C@H](O)[C@@H]2O)[C@@H](O)[C@H]1O] | Native Ligand |
| PARG-025 | −71 | 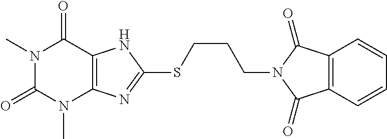 [SMILES: Cn1c(=O)c2[nH]c(SCCCN3C(=O)c4ccccc4C3=O)nc2n(C)c1=O] | Adenosyl |
| PARG-025 | −71 | 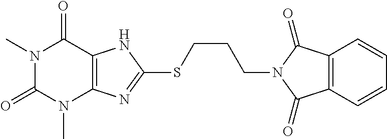 [SMILES: Cn1c(=O)c2[nH]c(SCCCN3C(=O)c4ccccc4C3=O)nc2n(C)c1=O] | Adenosyl |
| PARG-129 | −70 | 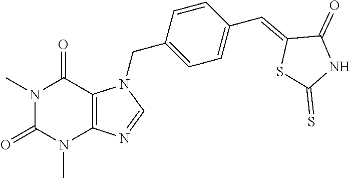 [SMILES: Cn1c(=O)c2c(ncn2Cc2ccc(/C=C3\SC(=S)NC3=O)cc2)n(C)c1=O] | Adenosyl |
| PARG-305 | −66 | 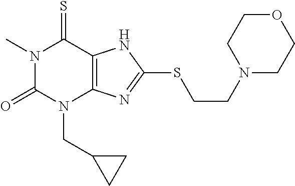 [SMILES: Cn1c(=S)c2[nH]c(SCCN3CCOCC3)nc2n(CC2CC2)c1=O] | Ribose |
| PARG-038 | −66 | 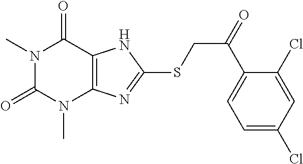 [SMILES: Cn1c(=O)c2[nH]c(SCC(=O)c3ccc(Cl)cc3Cl)nc2n(C)c1=O] | Ribose |
| PARG-129 | −65 | 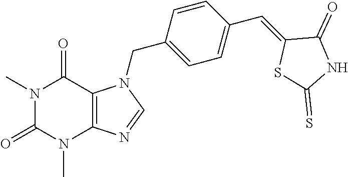 [SMILES: Cn1c(=O)c2c(ncn2Cc2ccc(/C=C3\SC(=S)NC3=O)cc2)n(C)c1=O] | Adenosyl |
| PARG-257 | −65 | 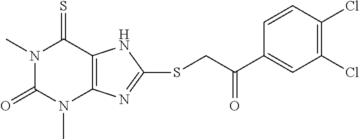 [SMILES: Cn1c(=S)c2[nH]c(SCC(=O)c3ccc(Cl)c(Cl)c3)nc2n(C)c1=O] | Ribose |
| PARG-108 | −64 | 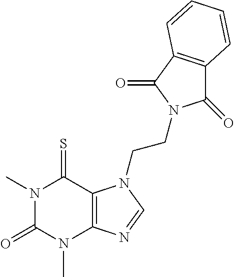 [SMILES: Cn1c(=S)c2c(ncn2CCN2C(=O)c3ccccc3C2=O)n(C)c1=O] | Ribose |
| PARG-120 | −63 | 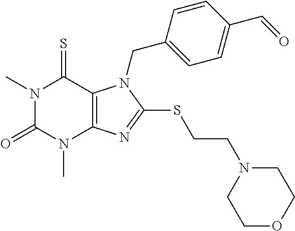 [SMILES: Cn1c(=S)c2c(nc(SCCN3CCOCC3)n2Cc2ccc(C=O)cc2)n(C)c1=O] | Ribose |
| PARG-066 | −63 | 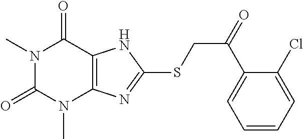 [SMILES: Cn1c(=O)c2[nH]c(SCC(=O)c3ccccc3Cl)nc2n(C)c1=O] | Ribose |
| PARG-066 | −63 | 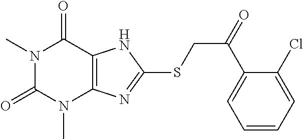 [SMILES: Cn1c(=O)c2[nH]c(SCC(=O)c3ccccc3Cl)nc2n(C)c1=O] | Ribose |
| PARG-033 | −63 | 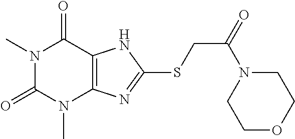 [SMILES: Cn1c(=O)c2[nH]c(SCC(=O)N3CCOCC3)nc2n(C)c1=O] | Ribose |
| PARG-038 | −61 | 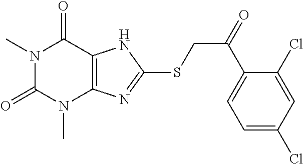 [SMILES: Cn1c(=O)c2[nH]c(SCC(=O)c3ccc(Cl)cc3Cl)nc2n(C)c1=O] | Ribose |
| PARG-252 | −60 | 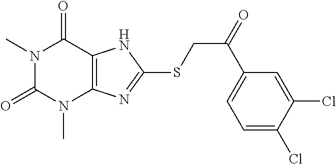 [SMILES: Cn1c(=O)c2[nH]c(SCC(=O)c3ccc(Cl)c(Cl)c3)nc2n(C)c1=O] | Ribose |
| PARG-258 | −60 | 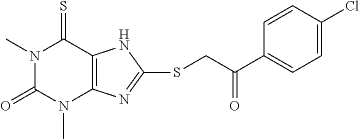 [SMILES: Cn1c(=S)c2[nH]c(SCC(=O)c3ccc(Cl)cc3)nc2n(C)c1=O] | Ribose |
| PARG-013 | −60 | 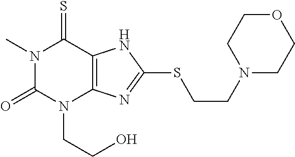 [SMILES: Cn1c(=S)c2[nH]c(SCCN3CCOCC3)nc2n(CCO)c1=O] | Ribose |
| PARG-272 | −60 | 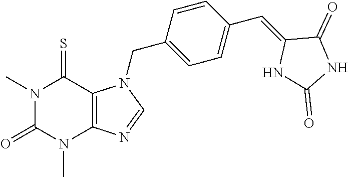 [SMILES: Cn1c(=S)c2c(ncn2Cc2ccc(/C=C3\NC(=O)NC3=O)cc2)n(C)c1=O] | Adenosyl |
| PARG-025 | −60 | 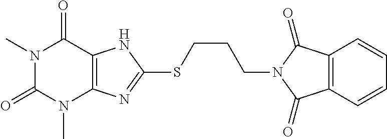 [SMILES: Cn1c(=O)c2[nH]c(SCCCN3C(=O)c4ccccc4C3=O)nc2n(C)c1=O] | Adenosyl |
| PARG-120 | −60 | 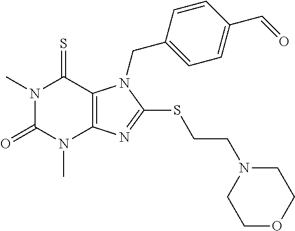 [SMILES: Cn1c(=S)c2c(nc(SCCN3CCOCC3)n2Cc2ccc(C=O)cc2)n(C)c1=O] | Ribose |

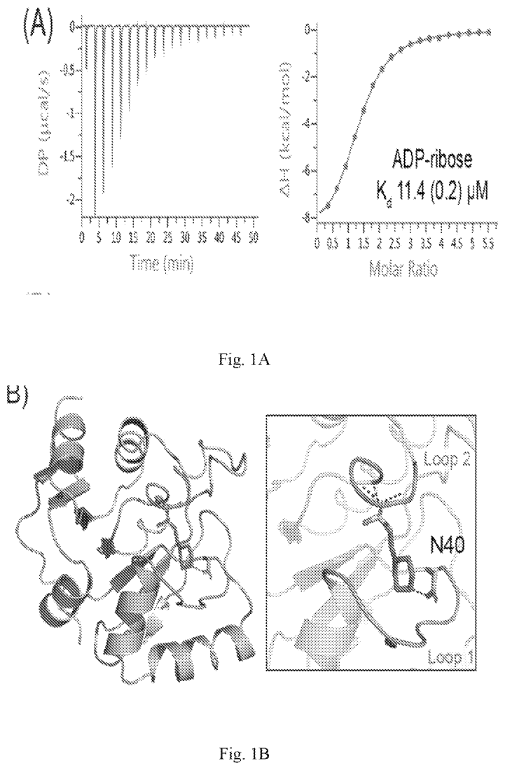

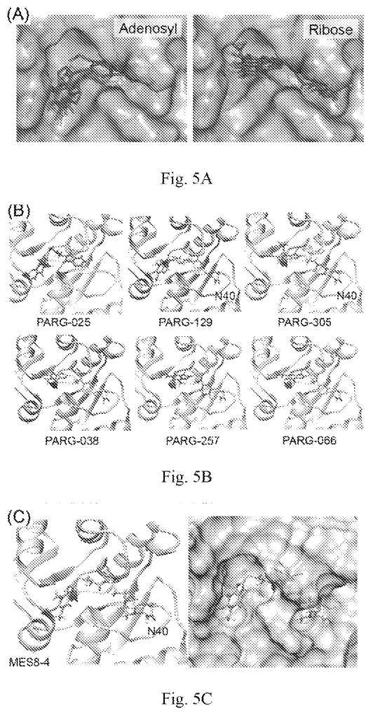

Visual inspection of the top twenty docked PARGi complexes reveals two clusters of poses. The first is enriched for C8 phenyl and morpholine derivatives that anchor into the distal ribose site (FIG. 5A). A second, smaller cluster, which contains the top two PARGi poses, anchors the methylxanthine head into the adenine pocket. Inspection of individual poses highlights contacts between the Loop 2 main chain and the conserved N40 of the distal ribose site, similar to those observed for the crystallographic MES ligand (FIG. 1). Notably, neither compound cluster fully bridges and engages both pockets of the active site, suggesting that extension of the JA2131 scaffold could be required to adapt these derivatives to CoV-2 Mac1. Nonetheless, the virtual ‘triaging’ provided by these results identifies PARGi fragment templates likely to be most useful in CoV-2 Mac2 inhibitor design.

### Results and Discussion

### Optimized MES8-4 (PARG-345) and PARG-329 Engage the CoV-2 Mac1 Active Site

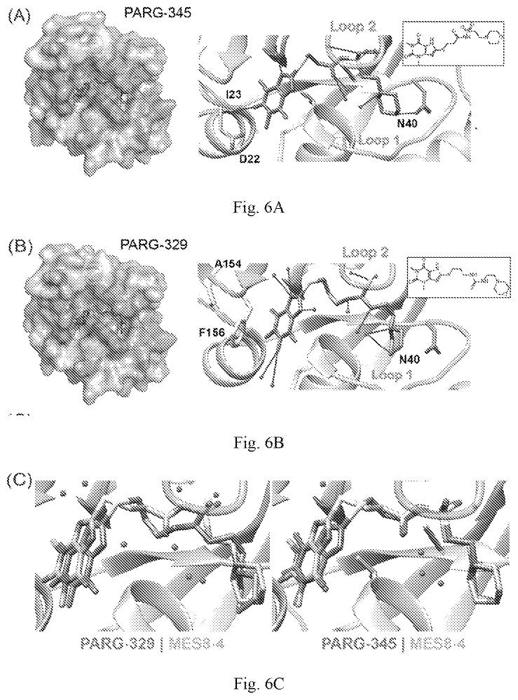

Using the virtual screening results as a guide, a panel of MES7 and MES8 derivatives was synthesized for binding assessment and crystallization with CoV-2 Mac1. As predicted, N7 variations of the MESi scaffold (Table 2-3) did not engage the Mac1 domain (data not shown). However, MES8-4 (PARG-345) and PARG-329, a variant of the MES8-4 scaffold that substitutes a thiourea for the acyl sulfonamide linker, successfully crystallized with the CoV-2 Mac1 domain at 2.3 Å and 1.55 Å, respectively (FIG. 6A, FIG. 6B, and FIG. 6C).

The backbone traces of these new Mac1 ligand complexes are highly similar to the MES crystal structure (PARG-345 Ca RMSD 0.169, PARG-329 Ca RMSD 0.358 Å) and closely mirror the conformation predicted by the MES8-4 computational studies, anchoring the methylxanthine head in the adenine pocket and the C8 morpholine in the terminal ribose pocket (FIG. 6C). Remarkably, the PARG-345 complex captures the major interactions observed in the original MVES8-4 computational model (methylxanthine-I23, sulfonyl-Loop 2, morpholine-N40) (FIG. 6A and FIG. 6C). In contrast, the PARG-329 ligand makes more water-mediated contacts with the macrodomain and adopts a strained conformation to fit into the Mac1 active site (FIG. 6B).

TABLE 2-3
First Generation CoV-2 Mac1 Inhibitors Based on PARGi Templates

| Name | Structure |
|---|---|
| MES7-1 | 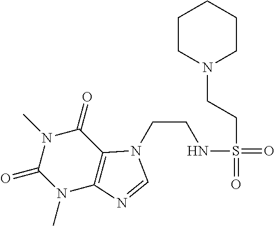 [SMILES: Cn1c(=O)c2c(ncn2CCNS(=O)(=O)CCN2CCCCC2)n(C)c1=O] |
| MES7-2 | 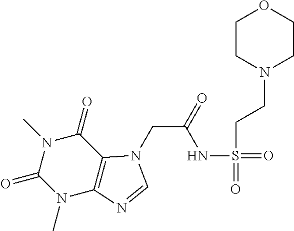 [SMILES: Cn1c(=O)c2c(ncn2CC(=O)NS(=O)(=O)CCN2CCOCC2)n(C)c1=O] |
| MES7-3 | 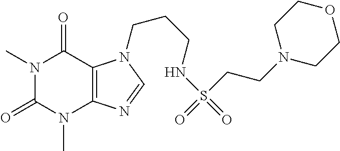 [SMILES: Cn1c(=O)c2c(ncn2CCCNS(=O)(=O)CCN2CCOCC2)n(C)c1=O] |
| MES7-4 | 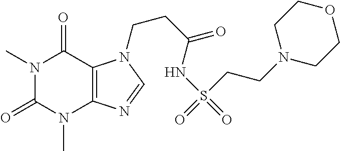 [SMILES: Cn1c(=O)c2c(ncn2CCC(=O)NS(=O)(=O)CCN2CCOCC2)n(C)c1=O] |
| MES8-1 | 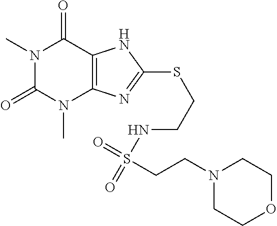 [SMILES: Cn1c(=O)c2[nH]c(SCCNS(=O)(=O)CCN3CCOCC3)nc2n(C)c1=O] |
| MES8-2 | 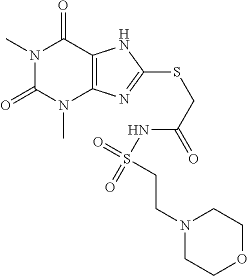 [SMILES: Cn1c(=O)c2[nH]c(SCC(=O)NS(=O)(=O)CCN3CCOCC3)nc2n(C)c1=O] |
| MES8-3 | 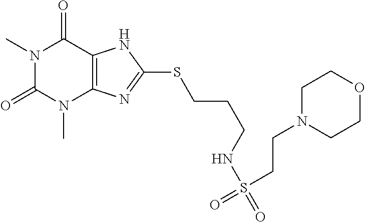 [SMILES: Cn1c(=O)c2[nH]c(SCCCNS(=O)(=O)CCN3CCOCC3)nc2n(C)c1=O] |
| MES8-4 | 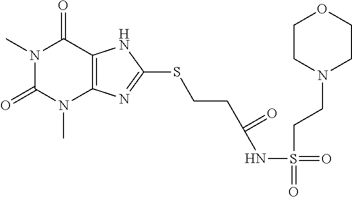 [SMILES: Cn1c(=O)c2[nH]c(SCCC(=O)NS(=O)(=O)CCN3CCOCC3)nc2n(C)c1=O] |

Both Mac1 ligand linkers extend into and engage Loop 2. The PARG-329 thiourea makes two water-mediated contacts to the Loop 2 main chain, while the PARG-345 sulfonyl makes two backbone and one water-mediated contact with Loop 2, consistent with interactions observed in the MES8-4 model. While the Mac1 ligand linkers exhibit comparable binding interactions, the methylxanthine and morpholine groups display individual variations. The methylxanthine head is rotated 180 degrees between PARG-329 and PARG-345. In this configuration, 03 of the PARG-345 methylxanthine directly hydrogen bonds with the 123 amide of the adenosyl pocket, while the PARG-329 methylxanthine remains engaged in water-mediated contacts. This variability in methylxanthine orientation echoes the variable adenine rotation of the Mac1/ADPr docking studies. F156, which has the potential to make 71-stacking interactions with the methylxanthine, remains imperfectly aligned for direct 71-to-71 and edge-to-face contact. Further extension of the ligand linker could enable the methylxanthine to orient for direct stacking, providing one avenue for further ligand optimization.

The morpholine groups of each ligand also vary in their active site engagement. The PARG-345 morpholine assumes the low-energy chair conformation, allowing the terminal oxygen to hydrogen bond with the critical N40 side chain, as predicted by the MES8-4 computational modeling. In contrast, the PARG-329 morpholine oxygen is kinked away from N40 and hydrogen bonds with a water. The 3-atom spacing between the morpholine and thiourea appears to force the morpholine into the higher-energy conformation in order to fit into the active site. This restricted positioning of the PARG-329 morpholine prevents optimal interaction with N40, which is expected to be a critical contact for ligand engagement.

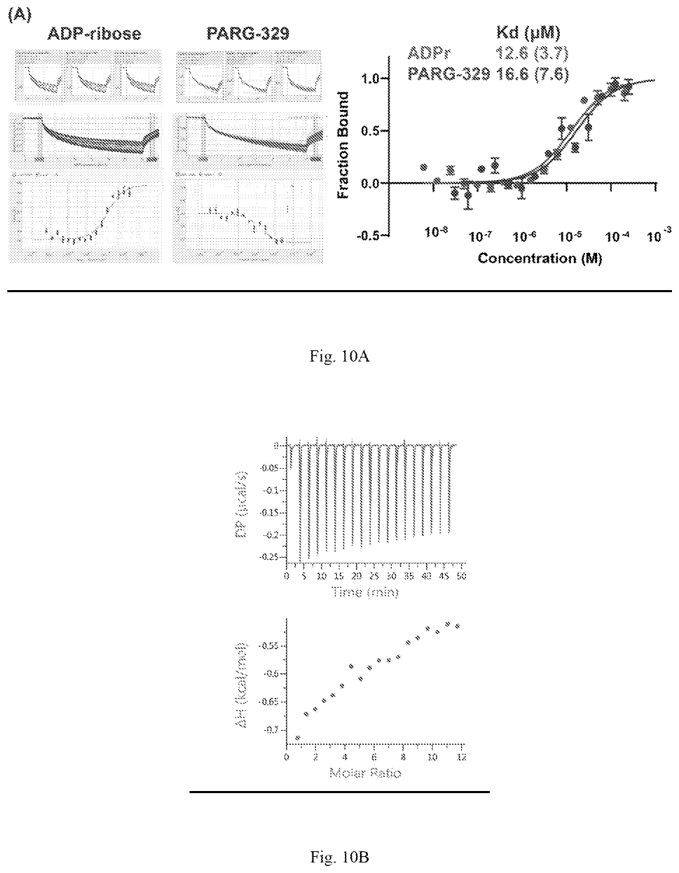

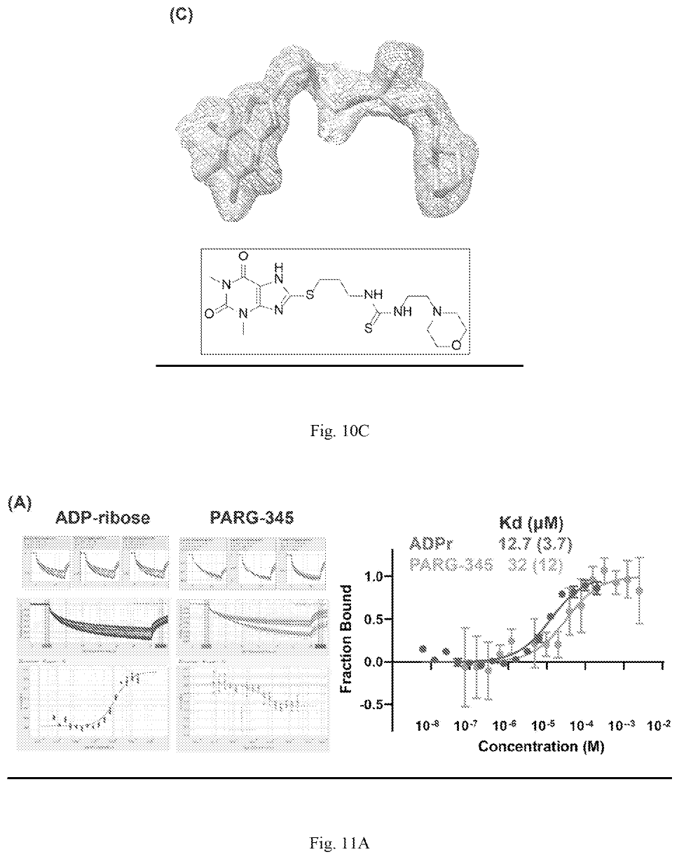

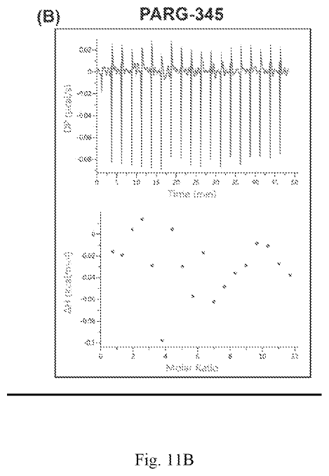

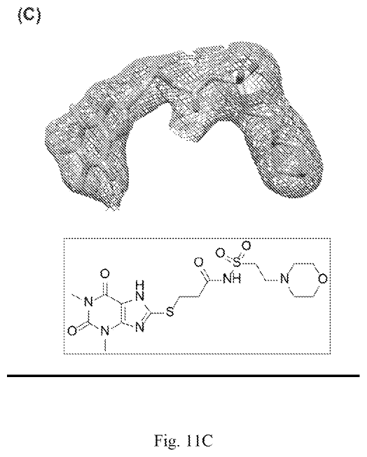

Preliminary measurements of binding between the Mac1 domain and these ligands by MST are suggestive of micromolar affinity (PARG-329, FIG. 10A, and FIG. 10B and PARG-345, FIGS. 11A, and 11B) however, ITC analysis fails to detect a robust interaction. The prevalence of water-mediated contacts and strained ligand conformation observed in the PARG-329 crystal structure would be consistent with weaker engagement of the active site. However, the prevalence of direct contacts between Mac1 and the PARG-345 ligand, as well as the more optimal ligand conformation, would be expected to produce higher binding affinity. A possible explanation for this discrepancy could be instability in the PARG-345 sulfonamide linker. Protonation of the morpholine nitrogen atom (pKa ˜8.3) under assay conditions (pH 7.5-8.5) could facilitate β-elimination of the morpholine, depleting the intact ligand and producing weaker than expected Mac1 binding.

### Rational Design and Modeling of COV-2 Mac1 Inhibitors Based Upon PARGi Fragments.

Direct MST and ITC affinity measurements between purified CoV-2 Mac1 and top PARGi candidates from the in silico screen revealed an absence of binding (data not shown), supporting a rationale for reconfiguring and elongating PARGi fragments to target both adenosyl and ribose pockets of the CoV-2 Mac 1 active site. These designs incorporate the 2-morpholinoethanesulfonyl moiety, the major fragment of MES, into the linker allowing the sulfone functional group to act as a bioisosteric replacement of ADPr phosphate groups in order to leverage the Loop 2 backbone contacts observed in the crystal structure and in silico screening (Table 2-3) (Elliott et al., 2012). To assess the ability of these first-generation CoV-2-specific inhibitors to bridge the ADP-ribose binding site, these ligands were computationally docked into the active site and ranked the resulting complexes in the context of the PARGi in silico results. Only poses for MES8-2 and MES8-4 were returned among the top docking results. Without being bound by theory, this suggests that inclusion of the carbonyl functionality of the acyl sulfonamide increases the C—N—S bond angle ˜10° and aids in optimally positioning the bridging sulfone to engage with Loop 2 (FIG. 5C and Table 2-3). The resulting model of the macrodomain/inhibitor complex for MES8-4 captures the ligand fully extended across both pockets of the active site, engaging conserved N40 with its morpholine group, the Loop 2 main chain with the bridging sulfone, and a novel 123 main chain contact in the adenosyl pocket with the methylxanthine head.

### Example 3: Synthesis of Compounds of Formula (Ia) or Formula (Ib)

Solvents and chemicals were reagent grade or better and obtained from commercial sources. Air and moisture sensitive reactions were carried out in oven-dried (at 120° C.) glassware. ¹H, ¹³C, and ¹⁹F NMR spectra were recorded using a 400 MHz NMR spectrometer. Sample purities were determined by HPLC analysis equipped with a mass spectrometric detector using a C18 3.5 μm, 30×2.1 mm column, eluting with a gradient system of 5:95 to 95:5 acetonitrile:water with a buffer consisting of 0.1% TFA over 4.5 min at 1 mL/min and detected by DAD. Analytical Thin Layer Chromatography (TLC) was performed on Merck silica gel plates (Merck Kieselgel 60, 0.25 mm thickness) with F254 indicator. Compounds were visualized under UV lamp or by developing in iodine. Medium pressure liquid chromatography (MPLC) separations were carried out using commercially available columns and technical grade solvents.

¹H NMR spectra were reported in ppm from tetranethylsilane (TMS) on the δ scale. Data are reported as follows: chemical shift, multiplicity (s=singlet, d=doublet, t=triplet, etc.; 3=multiplet, br=broadened, p=pentet), coupling constants (Hz), and assignments or relative integration where appropriate. ¹³C NMR spectra were reported in ppm from the central deuterated solvent peak (multiplicities indicated when determined). Grouped shifts are provided where an ambiguity has not been resolved.

### Synthesis of Methylxanthine PARGi Derivatives and Mac1 MES-Derived Inhibitors

The lead NO library compound JA2131 (NSC99667) was previously identified and characterized as a PARG inhibitor in vitro and in cells (Houl et al., 2019).

### General Procedure for the Preparation of N7-Substituted Methylxanthine Derivatives

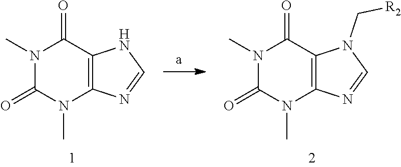

Preparation of N7-substituted methylxanthine PARGi derivatives. Reagents and conditions: (a) alkyl halide, K₂CO₃, DMF, 90° C., 12 h.

A general scheme for the preparation of N7-substituted methylxanthine PARGi 2 is generally known in the art (Bertrand et al., 2014). Commercially available theophylline 1 is treated with a variety of alkyl halides in the presence of potassium carbonate and affords N7-substituted methylxanthine PARGi 2 analogues in good yield and purity after chromatography.

### Preparation of Compound PARG-131

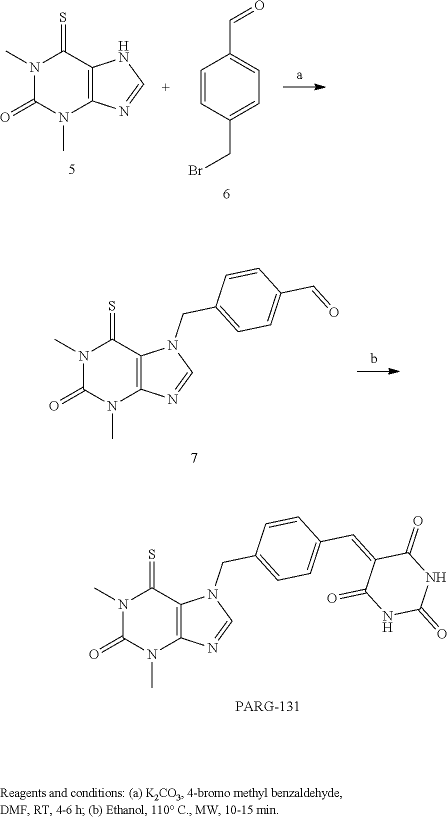 [SMILES: Cn1c(=S)c2[nH]cnc2n(C)c1=O ; O=Cc1ccc(CBr)cc1 ; Cn1c(=S)c2c(ncn2Cc2ccc(C=O)cc2)n(C)c1=O ; Cn1c(=S)c2c(ncn2Cc2ccc(C=C3C(=O)NC(=O)NC3=O)cc2)n(C)c1=O]

### Synthesis of 4-((1,3-dimethyl-2-oxo-6-thioxo-1,2,3,6-tetrahydro-7H-purin-7-yl)methyl)benzaldehyde (7)

Thioxanthine 5 (1.02 mmol) was taken into a round bottom flask with dry DMF (10 mL) under nitrogen atmosphere, potassium carbonate (3.06 mmol) was added and allowed it to stir for 15 min, then again 4-bromomethyl benzaldehyde 6 (1.02 mmol) was added, continued the reaction mass stirring for another 4 h. After completion of reaction, reaction mass was diluted with Ethyl acetate (10 mL) and filtered through the celite. The solvent was evaporated, purified by column chromatography to give the corresponding product 7 in 82% yield ¹H-NMR (400 MHz, CDCl₃) δ 9.99 (s, 1H), 7.87 (d, J=8.4 Hz, 2H), 7.70 (s, 1H), 7.34 (d, J=8.4 Hz, 2H), 6.06 (s, 2H), 3.80 (s, 3H), 3.66 (s, 3H); ¹³C-NMR (100 MHz, CDCl₃) δ: 191.6, 177.1, 150.1, 144.2, 142.9, 136.2, 130.4, 127.8, 49.9, 34.3, 30.5.

### Synthesis of 5-(4-((1,3-dimethyl-2-oxo-6-thioxo-1,2,3,6-tetrahydro-7H-purin-7-yl)methyl)benzylidene)pyrimidine-2,4,6(1H,3H,5H)-trione (PARG-131)

A mixture of thioxanthene aldehyde 7 (0.2 mmol) and barbituric acid (0.25 mmol) in 3 mL of ethanol was heated to 110° C. for 15 min under microwave irradiation. The resulting precipitate was washed with ether/dichloromethane (5 mL) to furnish the crude product. Purification by flash chromatography afforded pure PARG-131 as a keto/enol tautomer mixture: ¹H-NMR (400 MHz, DMSO-d6) δ 11.38 (s, 1H), 11.22 (s, 1H), 9.95 (s, 2H), 8.30 (s, 1H), 8.22 (d, J=8.8 Hz, 2H), 8.04 (d, J=8.4 Hz, 2H), 7.36 (d, J=8.4 Hz, 2H), 7.15 (d, J=7.6 Hz, 2H), 6.97 (d, J=7.6 Hz, 2H), 5.56 (s, 2H), 5.38 (s, 2H), 3.43 (s, 3H), 3.39 (s, 3H), 3.20 (s, 6H), 3.16 (s, 3H); ¹³C-NMR (100 MHz, DMSO-d6) δ: 192.7, 168.9, 163.3, 161.6, 154.5, 154.4, 154.0, 151.1, 150.2, 149.8, 148.6, 148.5, 143.5, 142.8, 142.7, 141.1, 135.7, 133.4, 132.2, 129.9, 127.9, 126.8, 119.2, 105.9, 84.9, 48.8, 29.5, 27.5.

### Preparation of Compound PARG-119

Prepared in similar fashion to compound PARG-131.

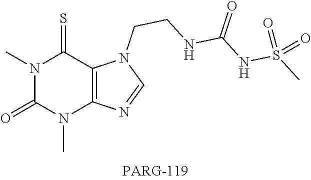 [SMILES: Cn1c(=S)c2c(ncn2CCNC(=O)NS(C)(=O)=O)n(C)c1=O]

NMR data for N-((2-(1,3-dimethyl-2-oxo-6-thioxo-1,2,3,6-tetrahydro-7H-purin-7-yl)ethyl)carbamoyl)methanesulfonamide (PARG-119): ¹H NMR (400 MHz, CDCl₃) δ 10.2 (s, 1H), 8.07 (s, 1H), 6.53 (brs, 1H), 4.69 (brs, 2H), 3.69 (s, 3H), 3.48 (s, 5H), 3.14 (s, 3H); ¹³C-NMR (100 MHz, CDCl₃) δ: 175.7, 152.4, 149.5, 146.3, 145.1, 116.9, 46.2, 41.3, 33.8, 30.1.

General Procedure for the Preparation of C8-substituted Methylxanthine PARGi Derivatives

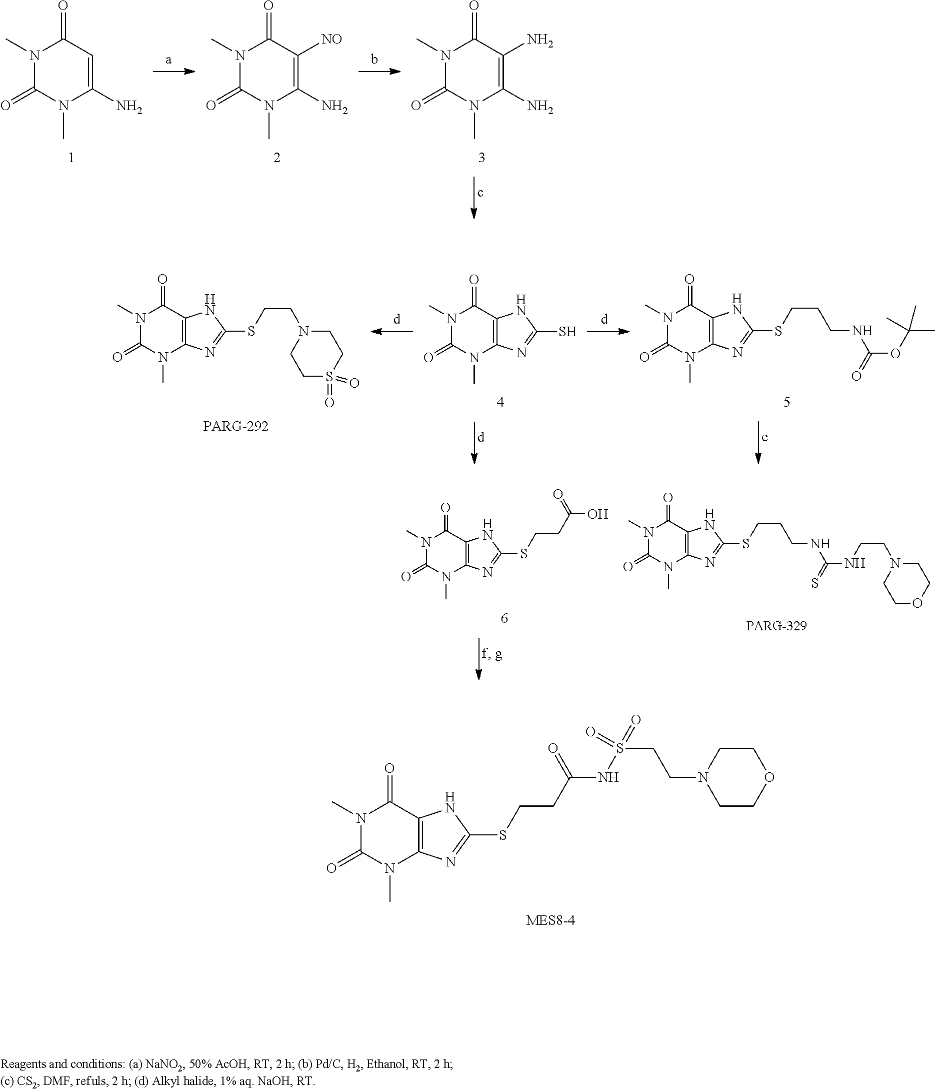

### Synthesis of 6-amino-1,3-dimethyl-5-nitrosuracil (2)

Aminouracil 1 (4.00 g, 25.8 mmol) was stirred in 50% aqueous AcOH solution (160 mL) at 75° C. for 30 minutes until the reaction mixture became homogeneous. Then the solution was cooled in an ice bath and NaNO₂ (3.56 g, 51.6 mmol) was added in small portions. The resulting purple solid was stirred for 1 hour. Finally, the purple solid was filtered, washed with water and dried in the vacuum to afford pure 6-amino-1,3-dimethyl-5-nitrosuracil 2 in 90% yield: ¹H NMR (400 MHz, DMSO-d6) δ 9.07 (s, 2H), 3.26 (s, 3H), 3.24 (s, 3H); ¹³C NMR (100 MHz, DMSO-d6) δ 160.3, 149.4, 146.1, 139.2, 28.7, 27.9.

### Synthesis of 5,6-diamino-1,3-dimethyluracil (3)

Compound 2 (2.00 g, 11.0 mmol) was sonicated with 100 mL of ethanol for 30 minutes. To another RBF equipped with a stir bar, Pd.C catalyst (5 mol %) was added and stirred with 100 mL ethanol. The catalyst solution was stirred under hydrogen atmosphere for 30 minutes. A suspension of compound 2 was then added to the catalyst solution via a canula and stirred at room temperature until completion (TLC 5% MeOH/DCM). The color of the solution changed from purple to black overtime. The solution was filtered through celite and the solvent was removed under reduced pressure to obtain 5,6-diamino-1,3-dimethyluracil 3 as a yellow solid in 68% yield. Compound 3 was used without further purification: ¹H NMR (400 MHz, DMSO-d6) δ 6.15 (s, 2H), 3.28 (s, 3H), 3.13 (s, 3H), 2.91 (s, 2H); ¹³C NMR (100 MHz, DMSO-d6) δ 159.0, 149.8, 144.7, 96.0, 29.8, 27.6.

### Synthesis of 8-thio-1,3-dimethyl-3,7-dihydro-1H-purine-2,6-dione (4)

Compound 3 (1.00 g, 6.0 mmol) was stirred with 100 mL of DMF in an round bottom flask equipped with a stir bar. The solution was stirred at room temperature until completely dissolved and then CS₂ (2.237 g, 30.0 mmol) was added to the mixture. The solution was refluxed for 6 hours. After confirming by TLC (5% MeOH/DCM), the solution was cooled in an ice bath and cold water was added, forming a white precipitate. The aqueous solution was filtered. The resulting solid was washed with cold water and ethyl ether to afford 8-thio-1,3-dimethyl-3,7-dihydro-1H-purine-2,6-dione 4 in 80% yield: ¹H NMR (400 MHz, DMSO-d6) δ 13.39 (s, 1H), 12.98 (s, 1H), 3.35 (s, 3H), 3.16 (s, 3H); ¹³C NMR (100 MHz, DMSO-d6) δ 163.9, 151.6, 150.1, 139.4, 103.6, 31.2, 27.8.

### Synthesis of 8-((2-(1,1-dioxidothiomorpholino)ethyl)thio)-1,3-dimethyl-3,7-dihydro-1H-purine-2,6-dione (PARG-292)

Thioxathine 4 (1.00 g, 4.71 mmol) was dissolved in a minimum volume of 1% NaOH until completely dissolved. In another dry vial, 4-(2-chloroethyl)thiomorpholine 1,1-dioxide (1.1 equiv.) was dissolved in a minimum volume of water. The alkyl halide solution was then added dropwise to the solution of 4 and stirred overnight at room temperature. After reaction completion, the crude solid product was filtered off, washed with water and dried. The crude product was purified using column chromatography (5% MeOH/Dichloromethane) solvent mixture to afford PARG-292 in 50% yield: ¹H NMR (400 MHz, CDCl₃) δ 13.66 (s, 1H), 3.58 (s, 3H), 3.44 (s, 3H), 3.32 (d, J=5.0 Hz, 4H), 3.27 (t, J=5.5 Hz, 2H), 3.21 (t, J=5.3 Hz, 4H), 3.09 (t, J=5.5 Hz, 2H); ¹³C NMR (100 MHz, CDCl₃) δ 154.8, 151.8, 150.5, 149.6, 108.6, 59.1, 51.6, 50.7, 30.4, 30.2, 28.5.

### Preparation of Compound PARG-002

Prepared in similar fashion to compound PARG-292.

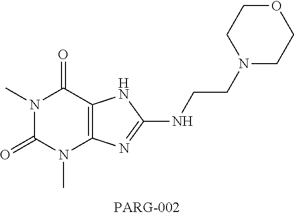 [SMILES: Cn1c(=O)c2[nH]c(NCCN3CCOCC3)nc2n(C)c1=O]

NMR data for 1,3-dimethyl-8-((2-morpholinoethyl)amino)-3,7-dihydro-1H-purine-2,6-dione (PARG-002): ¹H NMR (400 MHz, CDCl₃) δ 12.52 (s, 1H), 5.57 (s, 1H), 3.80 (s, 4H), 3.54 (s, 3H), 3.52 (d, J=5.0 Hz, 2H), 3.42 (s, 3H), 2.66 (t, J=5.0 Hz, 2H), 2.56 (s, 4H); ¹³C NMR (100 MHz, CDCl₃) δ 155.6, 154.2, 151.9, 150.6, 102.9, 66.9, 58.3, 53.6, 39.8, 30.2, 28.2.

### Preparation of Compound PARG-061

Prepared in similar fashion to compound PARG-292.

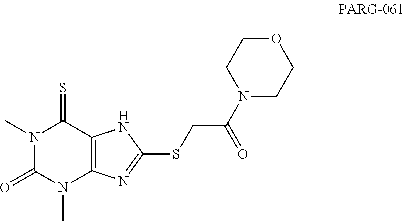 [SMILES: Cn1c(=S)c2[nH]c(SCC(=O)N3CCOCC3)nc2n(C)c1=O]

NMR data for 1,3-dimethyl-8-((2-morpholino-2-oxoethyl)thio)-6-thioxo-1,3,6,7-tetrahydro-2H-purin-2-one (PARG 061): ¹H NMR (400 MHz, DMSO-d6) δ 13.33 (s, 1H), 4.41 (s, 2H), 3.64 (s, 4H), 3.56 (s, 3H), 3.45 (s, 4H), 3.33 (s, 3H); ¹³C NMR (100 MHz, DMSO-d6) δ 172.6, 165.5, 152.9, 149.6, 143.8, 120.4, 66.0, 45.9, 42.1, 35.0, 33.9, 30.4.

### Preparation of Compound PARG-322

Prepared in similar fashion to compound PARG-292.

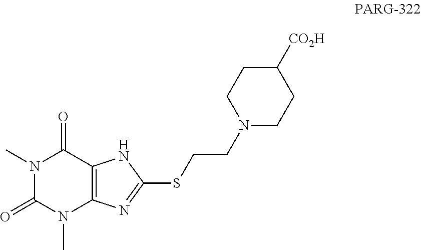 [SMILES: Cn1c(=O)c2[nH]c(SCCN3CCC(C(=O)O)CC3)nc2n(C)c1=O]

NMR data for 1-(2-((1,3-dimethyl-2,6-dioxo-2,3,6,7-tetrahydro-1H-purin-8-yl)thio)ethyl)piperidine-4-carboxylic acid (PARG-322): ¹H NMR (400 MHz, CD₃OD) δ 3.50 (s, 3H), 3.33 (s, 3H), 3.31-3.29 (m, 2H), 3.18 (t, J=6.0 Hz, 4H), 2.84 (s, 2H), 2.39 (d, J=4.1 Hz, 1H), 2.17 (s, 2H), 2.06-2.01 (m, 2H); ¹³C NMR (100 MHz, METHANOL-D3) δ 182.2, 158.6, 157.5, 153.6, 151.7, 114.9, 61.0, 53.9, 43.0, 30.9, 29.1, 28.5, 28.5.

### Preparation of Compound 5

Prepared in similar fashion to compound PARG-292.

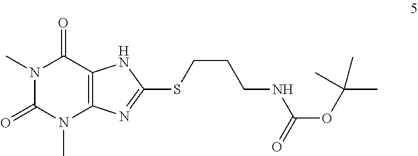 [SMILES: Cn1c(=O)c2[nH]c(SCCCNC(=O)OC(C)(C)C)nc2n(C)c1=O]

NMR data for tert-butyl (3-((1,3-dimethyl-2,6-dioxo-2,3,6,7-tetrahydro-1H-purin-8-yl)thio)propyl)carbamate (5). From method A. ¹H-NMR (400 MHz, CDCl₃) δ 3.72 (t, J=4.0 Hz, 4H), 3.59 (s, 3H), 3.40 (s, 3H), 3.28 (t, J=6.8 Hz, 2H), 3.25 (br's 4H), 2.61 (t, J=6.0 Hz, 2H), 2.54 (t, J=4.0 Hz, 4H), 2.06 (p, J=6.8 Hz, J=6.8 Hz, 2H); ¹³C-NMR (100 MHz, CDCl₃) δ 181.2, 154.5, 151.6, 149.7, 149.2, 108.4, 66.7, 53.4, 49.9, 40.8, 41.2, 30.2, 29.2, 28.2.

### Preparation of Compound PARG-329

Prepared in similar fashion to compound PARG-292.

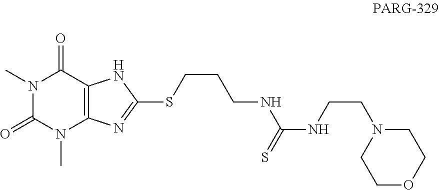 [SMILES: Cn1c(=O)c2[nH]c(SCCCNC(=S)NCCN3CCOCC3)nc2n(C)c1=O]

To the stirred solution of tert-butyl 3-(1,3-dimethyl-2,6-dioxo-2,3,6,7-tetrahydro-1H-purin-8-ylthio)propylcarbamate 5 (0.3 mmol) in dry DCM (2 mL) under nitrogen atmosphere trifluoroacetic acid (TFA) (2 mL) was added under cooling and allowed to stir it for 2 h at room temperature. After completion of the reaction, TFA was removed under reduced pressure; the resulting reaction mixture was re-dissolved in DCM, and undercooling, excess trimethylamine (2 mL) was added, followed by isocyanate (0.32 mmol), and continued stirring it for another 10 h. After completion of the reaction, DCM was evaporated, purified by column chromatography (20% MeOH/DCM) to give the desired product in 63% yield. ¹H-NMR (400 MHz, CDCl₃) δ 3.72 (t, J=4.0 Hz, 4H), 3.59 (s, 3H), 3.40 (s, 3H), 3.28 (t, J=6.8 Hz, 2H), 3.25 (br's 4H), 2.61 (t, J=6.0 Hz, 2H), 2.54 (t, J=4.0 Hz, 4H), 2.06 (p, J=6.8 Hz, J=6.8 Hz, 2H); ¹³C-NMR (100 MHz, CDCl₃) δ 181.2, 154.5, 151.6, 149.7, 149.2, 108.4, 66.7, 53.4, 49.9, 40.8, 41.2, 30.2, 29.2, 28.2.

### Preparation of Compound 6

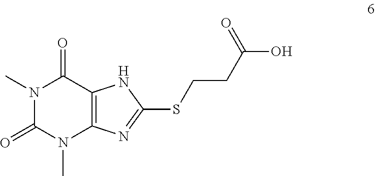 [SMILES: Cn1c(=O)c2[nH]c(SCCC(=O)O)nc2n(C)c1=O]

NMR data for 3-((1,3-dimethyl-2,6-dioxo-2,3,6,7-tetrahydro-1H-purin-8-yl)thio)propanoic acid (6). From method A. ¹H-NMR (400 MHz, CDCl₃) δ 3.37 (s, 3H), 3.27 (t, J=7.2 Hz, 2H), 3.19 (s, 3H), 2.62 (t, J=6.8 Hz, 2H); ¹³C-NMR (100 MHz, CDCl₃) δ 173.6, 154.4, 151.1, 149.9, 149.3, 110.8, 35.7, 29.8, 27.5, 27.3.

### Preparation of 3-(1,3-dimethyl-2,4-dioxo-2,3,4,5-tetrahydro-1H-pyrrolo[3,2-d]pyrimidin-6-ylthio)-N-(vinylsulfonyl)propanamide

To the stirred suspension of 3-(1,3-dimethyl-2,4-dioxo-2,3,4,5-tetrahydro-1H-pyrrolo[3,2-d]pyrimidin-6-ylthio)propanoic acid 6 (0.39 mmol) in 20 mL vial with DMF (4 mL), ethenesulfonamide (0.43 mmol), 2-chloro-1-methylpyridinium iodide (0.47 mmol) were added followed by 4-dimethylaminopyridine (0.03 mmol). The resulting reaction mixture was stirred for 5 min at room temperature, and then triethylamine (1.19 mmol) was added and continued stirring at room temperature overnight. After completion of the reaction, DMF was removed under reduced pressure, purified by column chromatography to furnish the desired product 58% yield; ¹H-NMR (400 MHz, CDCl₃) δ 6.79 (dd, J=9.6 Hz, J=16.4 Hz 1H), 6.39 (d, J=16.4 Hz 1H), 6.01 (d, J=9.6 Hz 1H), 3.53 (s, 3H), 3.40-3.34 (m, 4H), 2.76 (t, J=6.4 Hz, 2H).

### Preparation of 3-((1,3-dimethyl-2,6-dioxo-2,3,6,7-tetrahydro-1H-purin-8-yl)thio)-N-((2-morpholinoethyl)sulfonyl)propanamide (MES8-4)

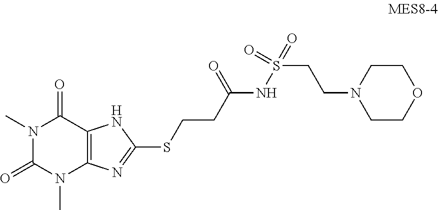 [SMILES: Cn1c(=O)c2[nH]c(SCCC(=O)NS(=O)(=O)CCN3CCOCC3)nc2n(C)c1=O]

To the stirred solution of 3-(1,3-dimethyl-2,4-dioxo-2,3,4,5-tetrahydro-1H-pyrrolo[3,2-d]pyrimidin-6-ylthio)-N-(vinylsulfonyl) propanamide (0.15 mmol) in DMF (2 mL), morpholine (0.15 mmol) in DMF (1 mL) was added slowly at room temperature and stirred for 8 h. Then, the DMF was removed under reduced pressure, purified by column chromatography (15% MeOH/DCM) to give the desired product in 36% yield; ¹H-NMR (400 MHz, CDCl₃) δ 3.72 (t, J=4.4 Hz, J=16.4 Hz 4H), 3.56 (s, 3H), 3.38 (s, 3H), 3.35 (t, J=6.0 Hz, 2H), 3.29 (t, J=7.6 Hz, 2H), 2.87 (t, J=7.6 Hz, 2H), 2.80 (t, J=6.4 Hz, 2H), 2.52 (t, J=4.4 Hz, 4H); ¹³C-NMR (100 MHz, CDCl₃) δ 154.7, 151.5, 150.9, 149.5, 108.2, 66.8, 53.4, 52.5, 51.0, 40.2, 30.3, 29.7, 28.2.

### Example 3: Crystallography of Cov-2 Mac1/Inhibitor Complexes

### Methods and Materials

### CoV-2 Mac1 Expression, Purification, and Validation

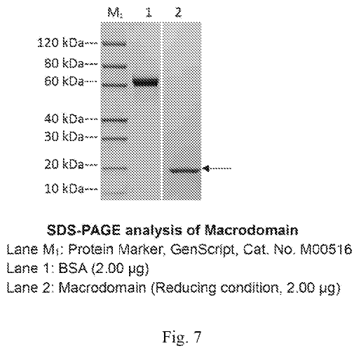

The CoV-2 Mac1 sequence (FIG. 7 and SEQ ID NO: 3) was subcloned into a pET-30a(+) vector with a 6X-Histidine fusion tag and 3C protease cleavage site by Genscript. CoV-2 Mac1 was recombinantly expressed in E. coli Rosetta2(DE3) cells using auto induction media at 20° C. Frozen cell pellets were resuspended on ice in lysis buffer (50 mM HEPES 8.0, 500 mM NaCl, 10% glycerol, 0.5 mM TCEP) supplemented with 1 mM PMSF, 1 mg/ml lysozyme, and 10 μg/ml DNase I. The suspension was lysed via 3 passes through an Avestin EmulsiFlex C5 Cell disruptor and spun at 18,000 rpm in a Sorvall SS-34 rotor for 60 min at 4° C. The supernatant was loaded onto a 5-ml GE HisTrap column, washed with 10 column volumes (CV) of lysis buffer, 5 CV of lysis buffer plus 25 mM imidazole, and eluted with lysis buffer plus 300 mM imidazole. Fractions containing the His₆-CoV-2 Mac1 were treated with precision protease and dialyzed overnight in 2 L of lysis buffer at 4° C. Subtractive chromatography was performed with the 5-ml GE HisTrap column, and the cleaved CoV-2 Mac1 product was eluted with lysis buffer plus 25 mM imidazole. The CoV-2 Mac1 was furthered purified on a Superdex 75 16/60 gel filtration column using 20 mM HEPES 7.5, 200 mM NaCl, and 0.5 mM TCEP. Fractions containing protein were pooled, flash frozen in liquid nitrogen, and stored at −80° C.

Purified CoV-2 Mac1 was assayed for its ability to bind ADP-ribose using isothermal titration calorimetry (ITC) with a PEAQ-ITC (Malvern Panalytical). Buffer-matched ADP-ribose stock (1 mM in 25 mM HEPES, pH 7.5, 150 mM NaCl) was titrated into buffer or 40-50 μM CoV-2 Mac1 at 25° C. in 19 2-μL injections (4 seconds each) spaced at 150 seconds and stirred at 750 rpm. ITC thermograms were baseline corrected and integrated, followed by subtraction of background heats from the ligand-to-buffer titration in MicroCal PEAQ-ITC Analysis Software. The same software was used to determine thermodynamic parameters.

### CoV-2 Mac1 Crystallization and Structure Determination

Crystals of CoV-2 Nsp3 Mac1 were grown by the hanging-drop vapor diffusion method in 70% saturated ammonium sulfate, 0.4% BME, 200 mM imidazole/malate pH 7.4, and 50 mM MES pH 6.0-8.6. Protein and mother liquor were mixed in a 1:1 ratio in 2 μL drops and equilibrated at 15° C. Crystals grew to full size in approximately five days. Crystals were harvested, briefly exchanged into cryoprotectant buffer (20% glycerol, i.e. 4 μL reservoir mixed with 1 μL glycerol), and flash cooled in liquid nitrogen for the native macrodomain structure. For PARG-329 complexes, crystals were soaked in 10 mM PARG-329 with 60% ammonium sulfate, 0.4% BME, 200 mM imidazole/malate, pH 7.8 for 5 hours prior to flash freezing in soak solution supplemented with 20% glycerol.

X-ray diffraction data were collected at SSRL beamline 12-2, and datasets were processed with XDS (Kabsch, 2010). The structure was solved with molecular replacement with Phaser, using PDB 6YWM, chain A as a search model. The structure was built with alternating rounds of refinement in Phenix (Liebschner et al., 2019) and manual fitting with Coot (Emsley and Cowtan, 2004). Final coordinates for CoV-2 Mac1 have been deposited in the Protein Data Bank (PDB: 7KG3). C_{α} RMSD values were calculated in Chimera (Pettersen et al., 2004).

### CoV-2 Mac1 MST Binding Affinity Measurements

MST measurements followed the framework outlined by (Seidel et al., 2013). Purified CoV-2 Mac1 was labeled with Atto488 NHS-ester (ATTO-TEC) according to the manufacturer's protocol with labeling efficiency 1:1 protein-to-dye ratio. Labeled CoV-2-Mac1 (100 nM) was combined with 1-4 mM PARGi in MST buffer (25 mM HEPES, pH 7.5, 150 mM NaCl, 0.01% Tween-20), incubated for 10-15 minutes at room temperature, and loaded into standard silica capillaries (NanoTemper). Microscale thermophoresis (MST) measurements were acquired on a Monolith NT.115 system (NanoTemper) at 25° C. with 30% LED power and 40% infrared excitation for 20 seconds with 5-second equilibration and recovery periods. Data were analyzed with Nano Temper analysis software.

### PARG Purification and Crystallization

The human PARG catalytic domain (residues 448-976) was recombinantly expressed and purified as described (Houl et al., 2019). Purified PARG was also crystallized as described (Houl et al., 2019; Tucker et al., 2012) by the hanging-drop vapor diffusion method in 0.1 M PCTP (Sodium propionate, Sodium cacodylate trihydrate, Bis-Tris propane), pH 7.5, 0.2 M AmSO₄, 18-23% PEG3350. PCTP buffer was obtained from Molecular Dimensions. Seeding was used to improve crystal yield and morphology (1.5 μL 7.5 mg/mL protein, 0.5 μL seed stock, 1.0 μL mother liquor). Crystals were harvested and soaked in 1-10 mM PARGi (prepared from 50 mM stocks in DMSO) for 1-2 hours at 22° C. in soak/cryoprotectant buffer (0.1 M PCTP, pH 7.5, 0.1 M NaCl, 0.15 M MgCl₂, 26% PEG3350, 2.5% glycerol), then flash cooled in liquid nitrogen for data collection. For PARG-131, PARG protein was mixed with 1-10 mM inhibitor and co-crystallized, resulting in a more primitive spacegroup (P12₁1) relative to the soaked, native crystals (P2₁2₁2₁). PARG-131 co-crystals were briefly soaked in cryoprotectant prior to flash cooling for data collection.

### PARGi Structure Determination

X-ray diffraction data were collected at Stanford Radiation Lightsource (SSRL) beamlines 9-2 and 12-2 (Russi et al., 2016; Soltis et al., 2008), National Synchrotron Light Source II (NSLS-II) beamlines 17-ID-1 (AMX) and 17-ID-2 (FMX), and Advanced Photon Source (APS) beamline 24-ID-C. X-ray data diffracted to 1.43-1.96 Å resolution and were processed with XDS (Kabsch, 2010) and the CCP4i suite (Winn et al., 2011). Structures with PARGi were determined by molecular replacement using unliganded PARG (PDB: 4B1G) as a search model in Phaser (Bunkoczi et al., 2013). PARGi structures were iteratively built using COOT (Emsley and Cowtan, 2004) and refined in Phenix (Liebschner et al., 2019). Ligand restraints were prepared with eLBOW (Moriarty et al., 2009). Polder difference maps were calculated in Phenix to validate PARGi ligand placement (Liebschner et al., 2017). Molecular images were generated with PyMOL (The PyMOL Molecular Graphics System, Version 2.0, Schrödinger, LLC). Final coordinates of PARGi complexes have been deposited in the Protein Data Bank [PARG-002 (7KG1), PARG-061 (7KG8), PARG-119 (7KFP), PARG-131 (7KG0), PARG-292 (7KG7), PARG-322 (7KG6)].

### Results

CoV-2 macrodomain 1 (Mac1) crystallizes with a PARGi template fragment in its active site.

To develop tools for PARGi repurposing, CoV-2 Mac1 was purified and crystallized, obtaining a 1.45 Å structure (FIG. 1A and FIG. 1B). The purified domain engages the native mono-nucleotide ADP-ribose ligand at a dissociation constant of K_{d} ˜10-12 μM, consistent with previously reported values (FIG. 1A and Table 3-1) (Frick et al., 2020). Examination of the CoV-2 Mac1 crystal structure revealed a canonical macrodomain fold with a molecule of MES captured within the distal ribose binding site, consistent with other recently reported structures of the CoV-2 macrodomain (Michalska et al., 2020). The Mac1 tertiary fold was nearly identical to the reported apo (C_{α} RMSD 0.386 Å, PDB: 6WEN), ADPr-bound (0.361 Å, PDB: 6W02), and MES-bound Mac1 structures (0.366 Å, PDB: 6WCF; 0.251 Å, PDB: 6YWM).

TABLE 3-1
CoV-2 Nsp3 Mac1 ITC Binding Parameters

| | | |
|---|---|---|
| N | 1.29 | (0.006) |
| Kd (μM) | 11.6 | (0.2) |
| ΔH (kcal/mol) | −9.18 | (0.2) |

| | |
|---|---|
| −TΔS (kcal/mol) | 2.43 |
| ΔG (kcal/mol) | −6.74 |

Fit errors are in parentheses.

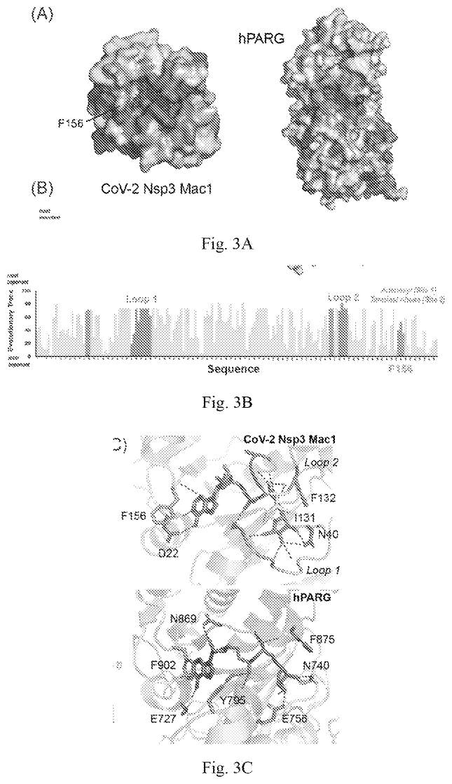

The MES ligand was coordinated by hydrogen bonding from N40 of Loop 1 to the morpholine oxygen and by Loop 2 main chain contacts to the sulfonic acid (FIG. 1). These interactions resemble those made by CoV-2 Mac1 to the β-phosphate and distal ribose of ADP-ribose (FIG. 2), suggesting that the MES fragment could serve as a template for inhibitor development. Notably, our previously published methylxanthine PARGi JA2131 (Houl et al., 2019) possesses a morpholine extension that could provide a ready-made template for CoV-2 Mac1 inhibitor development (FIG. 3A), offering impetus for a repurposing strategy.

### COV-2 Mac1 and Human PARG Macrodomains Share Critical Adenosyl and Ribose Residues.

While COV-2 Mac1 and human PARG occupy phylogenetically distinct branches within the macrodomain family, their active sites retain shared architectural elements which could support PARGi repurposing (FIG. 2B). Visual inspection of these domains in complex with ADP-ribose reveals a conserved aromatic interaction in the adenine binding pocket (F156, F902) and hydrogen bond contacts to N5 of the adenine ring (D22, E727). The canonical Loop 2 exhibits main chain hydrogen bond interactions to α- and β-phosphates of the substrate. The opposing distal ribose pocket is framed by F132/F875 and coordinates the 2′-OH and 3′-OH groups of the distal ribose through hydrogen bond contacts with Loop 1. The preference for main chain coordination in the CoV-2 Mac1 Loop 1 over PARG's selective side chain contacts points to the distinct substrates and chemistry executed by each domain.

In order to rank and prioritize active site regions and residues for computational PARGi screening, we performed Evolutionary Trace (ET) analyses for CoV-2 Mac1 and PARG (Lichtarge et al., 1996; Mihalek et al., 2004). We initially collected homologous sequences for each full-length protein, producing two non-overlapping trees unique to PARG and viral CoV-2 Nsp3 proteins, respectively. The ET analysis of these protein-specific trees prioritizes the active sites of CoV-2 Mac1 and PARG as the location of greatest functional importance within each protein fold (FIG. 3A). Inspection of viral Nsp3 ET values across the macrodomain sequence highlights F156 of the adenosyl binding site (F360 in full-length Nsp3) as a region of relative increased variability within the adenosyl and distal ribose sites (FIG. 3B). The uniqueness of phenylalanine in this position has been noted in other viral macrodomain sequence alignments (Alhammad and Fehr, 2020; Frick et al., 2020; Michalska et al., 2020) and could have functional implications for engaging host cell substrates.

The evolutionary importance of active site residues across the greater macrodomain family was assessed, represented by the curated, structure-aligned macrodomain sequences used in the phylogenetic analysis (FIG. 2C and Table 1-2). The resulting ET scores were also projected onto the CoV-2 Mac1 and PARG macrodomain structures (FIG. 2C). This broader analysis across multiple macrodomain subfamilies shows more diffuse ET signal across the active sites. However, residues that contribute side-chain specific contacts to ADP-ribose (noted earlier in structural comparison of the active sites) retain high ET values: F156, D22, N40 (CoV-2 Mac1) and F902, E727, N740, N869, Y795, E756 (PARG). Contacts mediated by main-chain interactions, such as Loops 1 and 2 at the distal ribose, are more likely to exhibit diminished ET values. Interestingly, the ET ranking of CoV-2 F156 is now increased when eukaryotic macrodomains are included in the analysis, reflecting the conservation of this residue among MacroD domains. Overall, the ET analysis supports selective inhibitor targeting at F156, D22, and N40 and would prioritize PARGi that engage these residues.

### REFERENCES

Abraham, R., McPherson, R. L., Dasovich, M., Badiee, M., Leung, A. K. L. and Griffin, D. E., 2020. Both ADP-Ribosyl-Binding and Hydrolase Activities of the Alphavirus nsP3 Macrodomain Affect Neurovirulence in Mice, mBio. 11, e03253-19.
Alhammad, Y. M. O. and Fehr, A. R., 2020. The Viral Macrodomain Counters Host Antiviral ADP-Ribosylation, Viruses. 12.
Altschul, S. F., Madden, T. L., Schäffer, A. A., Zhang, J., Zhang, Z., Miller, W. and Lipman, D. J., 1997. Gapped BLAST and PSI-BLAST: a new generation of protein database search programs, Nucleic Acids Res. 25, 3389-402.
Armougom, F., Moretti, S., Poirot, O., Audic, S., Dumas, P., Schaeli, B., Keduas, V. and Notredame, C., 2006. Expresso: automatic incorporation of structural information in multiple sequence alignments using 3D-Coffee, Nucleic Acids Res. 34, W604-8.
Bertrand, B., Stefan, L., Pirrotta, M., Monchaud, D., Bodio, E., Richard, P., Le Gendre, P., Warmerdam, E., de Jager, M. H., Groothuis, G. M., Picquet, M. and Casini, A., 2014. Caffeine-based gold(I) N-heterocyclic carbenes as possible anticancer agents: synthesis and biological properties, Inorg Chem. 53, 2296-303.
Blanco-Melo, D., Nilsson-Payant, B. E., Liu, W. C., Uhl, S., Hoagland, D., Møller, R., Jordan, T. X., Oishi, K., Panis, M., Sachs, D., Wang, T. T., Schwartz, R. E., Lim, J. K., Albrecht, R. A. and tenOever, B. R., 2020. Imbalanced Host Response to SARS-CoV-2 Drives Development of COVID-19, Cell. 181, 1036-1045.e9.
Brosey, C. A., Ho, C., Long, W. Z., Singh, S., Burnett, K., Hura, G. L., Nix, J. C., Bowman, G. R., Ellenberger, T. and Tainer, J. A., 2016. Defining NADH-Driven Allostery Regulating Apoptosis-Inducing Factor, Structure. 24, 2067-2079.
Bunkoczi, G., Echols, N., McCoy, A. J., Oeffner, R. D., Adams, P. D. and Read, R. J., 2013. Phaser.MRage: automated molecular replacement, Acta Crystallogr D Biol Crystallogr. 69, 2276-86.
Caprara, G., Prosperini, E., Piccolo, V., Sigismondo, G., Melacarne, A., Cuomo, A., Boothby, M., Rescigno, M., Bonaldi, T. and Natoli, G., 2018. PARP14 Controls the Nuclear Accumulation of a Subset of Type I IFN-Inducible Proteins, J Immunol. 200, 2439-2454.
Chen, S.-H. and Yu, X., 2019. Targeting dePARylation selectively suppresses DNA repair-defective and PARP inhibitor-resistant malignancies, Science Advances. 5, eaav4340.
Claverie, J. M., 2020. A Putative Role of de-Mono-ADP-Ribosylation of STAT1 by the SARS-CoV-2 Nsp3 Protein in the Cytokine Storm Syndrome of COVID-19, Viruses. 12.
Dai, M., Liu, D., Liu, M., Zhou, F., Li, G., Chen, Z., Zhang, Z., You, H., Wu, M., Zheng, Q., Xiong, Y., Xiong, H., Wang, C., Chen, C., Xiong, F., Zhang, Y., Peng, Y., Ge, S., Zhen, B., Yu, T., Wang, L., Wang, H., Liu, Y., Chen, Y., Mei, J., Gao, X., Li, Z., Gan, L., He, C., Li, Z., Shi, Y., Qi, Y., Yang, J., Tenen, D. G., Chai, L., Mucci, L. A., Santillana, M. and Cai, H., 2020. Patients with Cancer Appear More Vulnerable to SARS-CoV-2: A Multicenter Study during the COVID-19 Outbreak, Cancer Discov. 10, 783-791.
Eckelmann, B. J., Bacolla, A., Wang, H., Ye, Z., Guerrero, E. N., Jiang, W., El-Zein, R., Hegde, M. L., Tomkinson, A. E., Tainer, J. A. and Mitra, S., 2020. XRCC1 promotes replication restart, nascent fork degradation and mutagenic DNA repair in BRCA2-deficient cells, NAR Cancer. 2, zcaa013.
Edgar, R. C., 2004. MUSCLE: multiple sequence alignment with high accuracy and high throughput, Nucleic Acids Res. 32, 1792-7.
Elliott, T., Slowey, A., Ye, Y. and Conway, S., 2012. The use of phosphate bioisosteres in medicinal chemistry and chemical biology, MedChemComm. 3.
Emsley, P. and Cowtan, K., 2004. Coot: model-building tools for molecular graphics, Acta Crystallogr D Biol Crystallogr. 60, 2126-32.
Fehr, A. R., Athmer, J., Channappanavar, R., Phillips, J. M., Meyerholz, D. K. and Perlman, S., 2015. The nsp3 macrodomain promotes virulence in mice with coronavirus-induced encephalitis, J Virol. 89, 1523-36.
Fehr, A. R., Channappanavar, R., Jankevicius, G., Fett, C., Zhao, J., Athmer, J., Meyerholz, D. K., Ahel, I. and Perlman, S., 2016. The Conserved Coronavirus Macrodomain Promotes Virulence and Suppresses the Innate Immune Response during Severe Acute Respiratory Syndrome Coronavirus Infection, mBio. 7.
Fehr, A. R., Jankevicius, G., Ahel, I. and Perlman, S., 2018. Viral Macrodomains: Unique Mediators of Viral Replication and Pathogenesis, Trends Microbiol. 26, 598-610.
Felsenstein, J., 1985. Confidence limits on phylognies: An approach using the bootstrap Evolution. pp. 783-791.
Fraser, J. S., 2020. Identifying new ligands for the SARS-CoV-2 Macrodomain by Fragment Screening and Multi-temperature Crystallography (Version 1.0), Zenodo.
Frick, D. N., Virdi, R. S., Vuksanovic, N., Dahal, N. and Silvaggi, N. R., 2020. Molecular Basis for ADP-Ribose Binding to the Mac1 Domain of SARS-CoV-2 nsp3, Biochemistry.
Garcin, E. D., Arvai, A. S., Rosenfeld, R. J., Kroeger, M. D., Crane, B. R., Andersson, G., Andrews, G., Hamley, P. J., Mallinder, P. R., Nicholls, D. J., St-Gallay, S. A., Tinker, A. C., Gensmantel, N. P., Mete, A., Cheshire, D. R., Connolly, S., Stuehr, D. J., Aberg, A., Wallace, A. V., Tainer, J. A. and Getzoff, E. D., 2008. Anchored plasticity opens doors for selective inhibitor design in nitric oxide synthase, Nat Chem Biol. 4, 700-7.
Grunewald, M. E., Chen, Y., Kuny, C., Maejima, T., Lease, R., Ferraris, D., Aikawa, M., Sullivan, C. S., Perlman, S. and Fehr, A. R., 2019. The coronavirus macrodomain is required to prevent PARP-mediated inhibition of virus replication and enhancement of IFN expression, PLOS Pathogens. 15, e1007756.
Guan, W.-j., Ni, Z.-y., Hu, Y., Liang, W.-h., Ou, C.-q., He, J.-x., Liu, L., Shan, H., Lei, C.-., Hui, D. S. C., Du, B., Li, L.-j., Zeng, G., Yuen, K.-Y., Chen, R.-c., Tang, C.-A., Wang, T., Chen, P.-y., Xiang, J., Li, S.-y., Wang, J.-l., Liang, Z.-j., Peng, Y.-x., Wei, L., Liu, Y., Hu, Y.-h., Peng, P., Wang, J.-m., Liu, J.-y., Chen, Z., Li, G., Zheng, Z.-j., Qiu, S.-q., Luo, J., Ye, C.-j., Zhu, S.-y. and Zhong, N.-s., 2020. Clinical Characteristics of Coronavirus Disease 2019 in China, New England Journal of Medicine. 382, 1708-1720.
Han, S., Craig, J. A., Putnam, C. D., Carozzi, N. B. and Tainer, J. A., 1999. Evolution and mechanism from structures of an ADP-ribosylating toxin and NAD complex, Nature Structural Biology. 6, 932-936.
Han, S. and Tainer, J. A., 2002. The ARTT motif and a unified structural understanding of substrate recognition in ADP-ribosylating bacterial toxins and eukaryotic ADP-ribosyltransferases, Int J Med Microbiol. 291, 523-9.
Hisham, M., Youssif, B. G. M., Osman, E. E. A., Hayallah, A. M. and Abdel-Aziz, M., 2019. Synthesis and biological evaluation of novel xanthine derivatives as potential apoptotic antitumor agents, Eur J Med Chem. 176, 117-128.
Holder, T., Basquin, C., Ebert, J., Randel, N., Jollivet, D., Conti, E., Jékely, G. and Bono, F., 2013. Deep transcriptome-sequencing and proteome analysis of the hydrothermal vent annelid Alvinella pompejana identifies the CvP-bias as a robust measure of eukaryotic thermostability, Biol Direct. 8, 2.
Houl, J. H., Ye, Z., Brosey, C. A., Balapiti-Modarage, L. P. F., Namjoshi, S., Bacolla, A., Laverty, D., Walker, B. L., Pourfarjam, Y., Warden, L. S., Babu Chinnam, N., Moiani, D., Stegeman, R. A., Chen, M.-K., Hung, M.-C., Nagel, Z. D., Ellenberger, T., Kim, I.-K., Jones, D. E., Ahmed, Z. and Tainer, J. A., 2019. Selective small molecule PARG inhibitor causes replication fork stalling and cancer cell death, Nature communications. 10, 5654-5654.
Iwata, H., Goettsch, C., Sharma, A., Ricchiuto, P., Goh, W. W., Halu, A., Yamada, I., Yoshida, H., Hara, T., Wei, M., Inoue, N., Fukuda, D., Mojcher, A., Mattson, P. C., Barabasi, A. L., Boothby, M., Aikawa, E., Singh, S. A. and Aikawa, M., 2016. PARP9 and PARP14 cross-regulate macrophage activation via STAT1 ADP-ribosylation, Nat Commun. 7, 12849.
Jankevicius, G., Hassler, M., Golia, B., Rybin, V., Zacharias, M., Timinszky, G. and Ladurner, A. G., 2013. A family of macrodomain proteins reverses cellular mono-ADP-ribosylation, Nature structural & molecular biology. 20, 508-514.
Kabsch, W., 2010. XDS, Acta Crystallogr D Biol Crystallogr. 66, 125-32.
Kim, I.-K., Kiefer, J. R., Ho, C. M. W., Stegeman, R. A., Classen, S., Tainer, J. A. and Ellenberger, T., 2012. Structure of mammalian poly(ADP-ribose) glycohydrolase reveals a flexible tyrosine clasp as a substrate-binding element, Nature structural & molecular biology. 19, 653-656.
Kumar, S., Stecher, G., Li, M., Knyaz, C. and Tamura, K., 2018. MEGA X: Molecular Evolutionary Genetics Analysis across Computing Platforms, Molecular biology and evolution. 35, 1547-1549.
Lam, T. T.-Y., Jia, N., Zhang, Y.-W., Shum, M. H.-H., Jiang, J.-F., Zhu, H.-C., Tong, Y.-G., Shi, Y.-X., Ni, X.-B., Liao, Y.-S., Li, W.-J., Jiang, B.-G., Wei, W., Yuan, T.-T., Zheng, K., Cui, X.-M., Li, J., Pei, G.-Q., Qiang, X., Cheung, W. Y.-M., Li, L.-F., Sun, F.-F., Qin, S., Huang, J.-C., Leung, G. M., Holmes, E. C., Hu, Y.-L., Guan, Y. and Cao, W.-C., 2020. Identifying SARS-CoV-2-related coronaviruses in Malayan pangolins, Nature. 583, 282-285.
Lambrecht, M. J., Brichacek, M., Barkauskaite, E., Ariza, A., Ahel, I. and Hergenrother, P. J., 2015. Synthesis of dimeric ADP-ribose and its structure with human poly(ADP-ribose) glycohydrolase, J Am Chem Soc. 137, 3558-64.
Liang, W., Guan, W., Chen, R., Wang, W., Li, J., Xu, K., Li, C., Ai, Q., Lu, W., Liang, H., Li, S. and He, J., 2020. Cancer patients in SARS-CoV-2 infection: a nationwide analysis in China, Lancet Oncol. 21, 335-337.
Lichtarge, O., Bourne, H. R. and Cohen, F. E., 1996. An evolutionary trace method defines binding surfaces common to protein families, J Mol Biol. 257, 342-58. Liebschner, D., Afonine, P. V., Baker, M. L., Bunkoczi, G., Chen, V. B., Croll, T. I., Hintze, B., Hung, L. W., Jain, S., McCoy, A. J., Moriarty, N. W., Oeffner, R. D., Poon, B. K., Prisant, M. G., Read, R. J., Richardson, J. S., Richardson, D. C., Sammito, M. D., Sobolev, O. V., Stockwell, D. H., Terwilliger, T. C., Urzhumtsev, A. G., Videau, L. L., Williams, C. J. and Adams, P. D., 2019. Macromolecular structure determination using X-rays, neutrons and electrons: recent developments in Phenix, Acta Crystallogr D Struct Biol. 75, 861-877.
Liebschner, D., Afonine, P. V., Moriarty, N. W., Poon, B. K., Sobolev, O. V., Terwilliger, T. C. and Adams, P. D., 2017. Polder maps: improving OMIT maps by excluding bulk solvent, Acta Crystallogr D Struct Biol. 73, 148-157.
Lin, M. H., Chang, S. C., Chiu, Y. C., Jiang, B. C., Wu, T. H. and Hsu, C. H., 2020. Structural, Biophysical, and Biochemical Elucidation of the SARS-CoV-2 Nonstructural Protein 3 Macro Domain, ACS Infect Dis.
Lua, R. C. and Lichtarge, O., 2010. PyETV: a PyMOL evolutionary trace viewer to analyze functional site predictions in protein complexes, Bioinformatics. 26, 2981-2.
McPherson, R. L., Abraham, R., Sreekumar, E., Ong, S.-E., Cheng, S.-J., Baxter, V. K., Kistemaker, H. A. V., Filippov, D. V., Griffin, D. E. and Leung, A. K. L., 2017. ADP-ribosylhydrolase activity of Chikungunya virus macrodomain is critical for virus replication and virulence, Proceedings of the National Academy of Sciences. 114, 1666-1671.
Michalska, K., Kim, Y., Jedrzejczak, R., Maltseva, N. I., Stols, L., Endres, M. and Joachimiak, A., 2020. Crystal structures of SARS-CoV-2 ADP-ribose phosphatase: from the apo form to ligand complexes, IUCrJ. 7, 814-824.
Mihalek, I., Res, I. and Lichtarge, O., 2004. A family of evolution-entropy hybrid methods for ranking protein residues by importance, J Mol Biol. 336, 1265-82.
Moiani, D., Ronato, D. A., Brosey, C. A., Arvai, A. S., Syed, A., Masson, J. Y., Petricci, E. and Tainer, J. A., 2018. Targeting Allostery with Avatars to Design Inhibitors Assessed by Cell Activity: Dissecting MRE11 Endo- and Exonuclease Activities, Methods Enzymol. 601, 205-241.
Moriarty, N. W., Grosse-Kunstleve, R. W. and Adams, P. D., 2009. electronic Ligand Builder and Optimization Workbench (eLBOW): a tool for ligand coordinate and restraint generation, Acta Crystallogr D Biol Crystallogr. 65, 1074-80.
Morin, A., Eisenbraun, B., Key, J., Sanschagrin, P. C., Timony, M. A., Ottaviano, M. and Sliz, P., 2013. Collaboration gets the most out of software, Elife. 2, e01456.
Nei, M. a. K., S., 2000. Molecular Evolution and Phylogenetics, Oxford University Press, New York.
Notredame, C., Higgins, D. G. and Heringa, J., 2000. T-Coffee: A novel method for fast and accurate multiple sequence alignment, J Mol Biol. 302, 205-17.
Omolo, C. A., Soni, N., Fasiku, V. O., Mackraj, I. and Govender, T., 2020. Update on therapeutic approaches and emerging therapies for SARS-CoV-2 virus, Eur J Pharmacol. 883, 173348.
Pettersen, E. F., Goddard, T. D., Huang, C. C., Couch, G. S., Greenblatt, D. M., Meng, E. C. and Ferrin, T. E., 2004. UCSF Chimera—a visualization system for exploratory research and analysis, J Comput Chem. 25, 1605-12.
Plante, J. A., Liu, Y., Liu, J., Xia, H., Johnson, B. A., Lokugamage, K. G., Zhang, X., Muruato, A. E., Zou, J., Fontes-Garfias, C. R., Mirchandani, D., Scharton, D., Bilello, J. P., Ku, Z., An, Z., Kalveram, B., Freiberg, A. N., Menachery, V. D., Xie, X., Plante, K. S., Weaver, S. C. and Shi, P. Y., 2020. Spike mutation D614G alters SARS-CoV-2 fitness, Nature.
Pourfarjam, Y., Kasson, S., Tran, L., Ho, C., Lim, S. and Kim, I.-K., 2020. PARG has a robust endo-glycohydrolase activity that releases protein-free poly(ADP-ribose) chains, Biochemical and Biophysical Research Communications. 527, 818-823.
Pruitt, K. D., Tatusova, T. and Maglott, D. R., 2007. NCBI reference sequences (RefSeq): a curated non-redundant sequence database of genomes, transcripts and proteins, Nucleic acids research. 35, D61-5.
Rack, J. G. M., Palazzo, L. and Ahel, I., 2020a. (ADP-ribosyl)hydrolases: structure, function, and biology, Genes & Development. 34, 263-284.
Rack, J. G. M., Perina, D. and Ahel, I., 2016. Macrodomains: Structure, Function, Evolution, and Catalytic Activities, Annual Review of Biochemistry. 85, 431-454.
Rack, J. G. M., Zorzini, V., Zhu, Z., Schuller, M., Ahel, D. and Ahel, I., 2020b. Viral macrodomains: a structural and evolutionary assessment of the pharmacological potential, Open Biol. 10, 200237.
Russi, S., Song, J., McPhillips, S. E. and Cohen, A. E., 2016. The Stanford Automated Mounter: pushing the limits of sample exchange at the SSRL macromolecular crystallography beamlines, J Appl Crystallogr. 49, 622-626.
Saitou, N. and Nei, M., 1987. The neighbor-joining method: a new method for reconstructing phylogenetic trees, Molecular Biology and Evolution. 4, 406-425.
Seidel, S. A., Dijkman, P. M., Lea, W. A., van den Bogaart, G., Jerabek-Willemsen, M., Lazic, A., Joseph, J. S., Srinivasan, P., Baaske, P., Simeonov, A., Katritch, I., Melo, F. A., Ladbury, J. E., Schreiber, G., Watts, A., Braun, D. and Duhr, S., 2013. Microscale thermophoresis quantifies biomolecular interactions under previously challenging conditions, Methods. 59, 301-15.
Shibata, A., Moiani, D., Arvai, A. S., Perry, J., Harding, S. M., Genois, M. M., Maity, R., van Rossum-Fikkert, S., Kertokalio, A., Romoli, F., Ismail, A., Ismalaj, E., Petricci, E., Neale, M. J., Bristow, R. G., Masson, J. Y., Wyman, C., Jeggo, P. A. and Tainer, J. A., 2014. DNA double-strand break repair pathway choice is directed by distinct MRE11 nuclease activities, Mol Cell. 53, 7-18.
Shin, D. S., Didonato, M., Barondeau, D. P., Hura, G. L., Hitomi, C., Berglund, J. A., Getzoff, E. D., Cary, S. C. and Tainer, J. A., 2009. Superoxide dismutase from the eukaryotic thermophile Alvinella pompejana: structures, stability, mechanism, and insights into amyotrophic lateral sclerosis, J Mol Biol. 385, 1534-55.
Shyr, Z. A., Gorshkov, K., Chen, C. Z. and Zheng, W., 2020. Drug discovery strategies for SARS-CoV-2, Journal of Pharmacology and Experimental Therapeutics. JPET-MR-2020-000123.
Slade, D., 2020. PARP and PARG inhibitors in cancer treatment, Genes Dev. 34, 360-394.
Slade, D., Dunstan, M. S., Barkauskaite, E., Weston, R., Lafite, P., Dixon, N., Ahel, M., Leys, D. and Ahel, I., 2011. The structure and catalytic mechanism of a poly(ADP-ribose) glycohydrolase, Nature. 477, 616-620.
Soltis, S. M., Cohen, A. E., Deacon, A., Eriksson, T., Gonzilez, A., McPhillips, S., Chui, H., Dunten, P., Hollenbeck, M., Mathews, I., Miller, M., Moorhead, P., Phizackerley, R. P., Smith, C., Song, J., van dem Bedem, H., Ellis, P., Kuhn, P., McPhillips, T., Sauter, N., Sharp, K., Tsyba, I. and Wolf, G., 2008. New paradigm for macromolecular crystallography experiments at SSRL: automated crystal screening and remote data collection, Acta Crystallogr D Biol Crystallogr. 64, 1210-21.
Stanke, M. and Morgenstern, B., 2005. AUGUSTUS: a web server for gene prediction in eukaryotes that allows user-defined constraints, Nucleic Acids Res. 33, W465-7.
Stecher, G., Tamura, K. and Kumar, S., 2020. Molecular Evolutionary Genetics Analysis (MEGA) for macOS, Molecular Biology and Evolution. 37, 1237-1239.
Suzek, B. E., Wang, Y., Huang, H., McGarvey, P. B. and Wu, C. H., 2015. UniRef clusters: a comprehensive and scalable alternative for improving sequence similarity searches, Bioinformatics. 31, 926-32.
Syed, A. and Tainer, J. A., 2018. The MRE11-RAD50-NBS1 Complex Conducts the Orchestration of Damage Signaling and Outcomes to Stress in DNA Replication and Repair, Annu Rev Biochem. 87, 263-294.
Tillett, R. L., Sevinkay, J. R., Hartley, P. D., Kerwin, H., Crawford, N., Gorzalski, A., Laverdure, C., Verma, S. C., Rossetto, C. C., JAckson, D., Farrell, M. J., Van Hooser, S. and Pandori, M., 2020. Genomic evidence for reinfection with SARS-CoV-2: a case study, The Lancet Infectious Diseases. In Press.
Touret, F., Gilles, M., Barral, K., Nougairède, A., van Helden, J., Decroly, E., de Lamballerie, X. and Coutard, B., 2020. In vitro screening of a FDA approved chemical library reveals potential inhibitors of SARS-CoV-2 replication, Scientific Reports. 10, 13093.
Tucker, J. A., Bennett, N., Brassington, C., Durant, S. T., Hassall, G., Holdgate, G., McAlister, M., Nissink, J. W. M., Truman, C. and Watson, M., 2012. Structures of the human poly (ADP-ribose) glycohydrolase catalytic domain confirm catalytic mechanism and explain inhibition by ADP-HPD derivatives, PloS one. 7, e50889-e50889.
Wang, Y., Kim, N. S., Haince, J. F., Kang, H. C., David, K. K., Andrabi, S. A., Poirier, G. G., Dawson, V. L. and Dawson, T. M., 2011. Poly(ADP-ribose) (PAR) binding to apoptosis-inducing factor is critical for PAR polymerase-1-dependent cell death (parthanatos), Sci Signal. 4, ra20.
Webb, T. E. and Saad, R., 2020. Sequence homology between human PARP14 and the SARS-CoV-2 ADP ribose 1′-phosphatase, Immunol Lett. 224, 38-39.
Wilson, D. M., 3rd, Deacon, A. M., Duncton, M. A. J., Pellicena, P., Georgiadis, M. M., Yeh, A. P., Arvai, A. S., Moiani, D., Tainer, J. A. and Das, D., 2020. Fragment- and structure-based drug discovery for developing therapeutic agents targeting the DNA Damage Response, Prog Biophys Mol Biol.
Winn, M. D., Ballard, C. C., Cowtan, K. D., Dodson, E. J., Emsley, P., Evans, P. R., Keegan, R. M., Krissinel, E. B., Leslie, A. G., McCoy, A., McNicholas, S. J., Murshudov, G. N., Pannu, N. S., Potterton, E. A., Powell, H. R., Read, R. J., Vagin, A. and Wilson, K. S., 2011. Overview of the CCP4 suite and current developments, Acta Crystallogr D Biol Crystallogr. 67, 235-42.
Zandarashvili, L., Langelier, M. F., Velagapudi, U. K., Hancock, M. A., Steffen, J. D., Billur, R., Hannan, Z. M., Wicks, A. J., Krastev, D. B., Pettitt, S. J., Lord, C. J., Talele, T. T., Pascal, J. M. and Black, B. E., 2020. Structural basis for allosteric PARP-1 retention on DNA breaks, Science. 368.
Zhang, C., Wu, Z., Li, J. W., Zhao, H. and Wang, G. Q., 2020. Cytokine release syndrome in severe COVID-19: interleukin-6 receptor antagonist tocilizumab may be the key to reduce mortality, Int J Antimicrob Agents. 55, 105954.
Zhang, Y., Mao, D., Roswit, W. T., Jin, X., Patel, A. C., Patel, D. A., Agapov, E., Wang, Z., Tidwell, R. M., Atkinson, J. J., Huang, G., McCarthy, R., Yu, J., Yun, N. E., Paessler, S., Lawson, T. G., Omattage, N. S., Brett, T. J. and Holtzman, M. J., 2015. PARP9-DTX3L ubiquitin ligase targets host histone H2BJ and viral 3C protease to enhance interferon signaling and control viral infection, Nat Immunol. 16, 1215-27.
Zhou, P., Yang, X.-L., Wang, X.-G., Hu, B., Zhang, L., Zhang, W., Si, H.-R., Zhu, Y., Li, B., Huang, C.-L., Chen, H.-D., Chen, J., Luo, Y., Guo, H., Jiang, R.-D., Liu, M.-Q., Chen, Y., Shen, X.-R., Wang, X., Zheng, X.-S., Zhao, K., Chen, Q.-J., Deng, F., Liu, L.-L., Yan, B., Zhan, F.-X., Wang, Y.-Y., Xiao, G.-F. and Shi, Z.-L., 2020. A pneumonia outbreak associated with a new coronavirus of probable bat origin, Nature. 579, 270-273.

Having now fully described this invention, it will be understood by those of ordinary skill in the art that the same can be performed within a wide and equivalent range of conditions, formulations, and other parameters without affecting the scope of the invention or any aspect thereof.

Other aspects of the present disclosure will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A method for treating or reducing the coronavirus pathogenicity and the symptoms associated with a coronavirus infection, the method comprising administering to a subject in need thereof a therapeutically effective amount of a compound of Formula (Ia) or Formula (Ib): 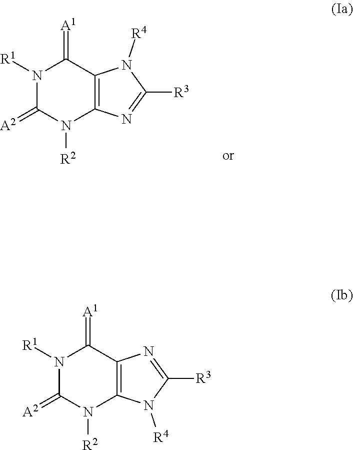
wherein:
A¹ and A² are independently 0, S, or NH;
R¹ and R² are independently unsubstituted C₁-C₆ alkyl, (hydroxy)C₁-C₆ alkyl, or (C₃-C₆ cycloalkyl)C₁-C₆ alkyl;
R³ is H or —X—Y—R⁵;
R⁴ is H or —Y′—R^{5′};
X is absent, S, O, or NH;
Y is —(CH₂)ₙ— or —(CH₂)ₘ(C═O)—;
n is an integer of 1, 2, 3, 4, 5, or 6;
m is an integer of 0, 1, 2, 3, 4, 5, or 6;
Y′ is —(CH₂)_{n′}— or —(CH₂)_{m′}(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo, 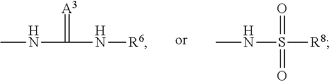
A³ is O, S, or NH;
R⁶ is optionally substituted C₁-C₆ alkyl, optionally substituted (3- to 10-membered heterocyclo)C₁-C₆ alkyl, or S(═O)₂R⁷;
R⁷ is optionally substituted C₁-C₆ alkyl or optionally substituted (3- to 10-membered heterocyclo)C₁-C₆ alkyl;
R⁸ is optionally substituted C₁-C₆ alkyl or optionally substituted (3- to 10-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo, 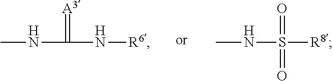
A^{3′} is O, S, or NH;
R^{6′} is optionally substituted C₁-C₆ alkyl, optionally substituted (3- to 10-membered heterocyclo)C₁-C₆ alkyl, or S(═O)₂R^{7′};
R^{7′} is optionally substituted C₁-C₆ alkyl or optionally substituted (3- to 10-membered heterocyclo)C₁-C₆ alkyl; and
R^{8′} is optionally substituted C₁-C₆ alkyl or optionally substituted (3- to 10-membered heterocyclo)C₁-C₆ alkyl;
or a pharmaceutically acceptable salt thereof,
with the proviso that R³ and R⁴ are not both H.

2. The method of claim 1, wherein the compound is a compound of Formula (Ia).

3. The method of claim 1, wherein the compound is a compound of Formula (Ib).

4. The method of any one of claims 1 to 3, wherein A¹ and A² are independently O or S.

5. The method of claim 4, wherein A¹ and A² are 0.

6. The method of any one of claims 1 to 5, wherein R¹ and R² are independently —CH₃, hydroxy(CH₂)₂—, or methylcyclopropyl.

7. The method of any one of claims 1 to 6, wherein R³ is H and R⁴ is —Y′—R^{5′}.

8. The method of claim 7, wherein:
R^{5′} is optionally substituted aryl, optionally substituted 3- to 9-membered heterocyclo, 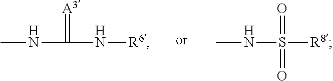
A^{3′} is O, S, or NH;
R^{6′} is optionally substituted C₁-C₆ alkyl or S(═O)₂R^{7′};
R^{7′} is optionally substituted C₁-C₆ alkyl or optionally substituted (3- to 6-membered heterocyclo)C₁-C₆ alkyl; and
R^{8′} is optionally substituted (3- to 6-membered heterocyclo)C₁-C₆ alkyl.

9. The method of claim 8, wherein:
n′ is 1 or 2;
R⁵ is optionally substituted aryl, optionally substituted 9-membered heterocyclo, or 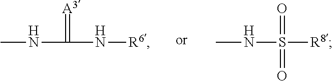
A^{3′} is O, S, or NH;
R^{6′} is S(═O)₂R⁷;
R^{7′} is optionally substituted C₁-C₆ alkyl; and
R^{8′} is optionally substituted (6-membered heterocyclo)C₁-C₆ alkyl.

10. The method of claim 9, wherein:
R^{5′} is 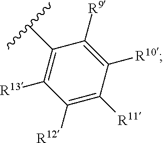
R^{9′}, R^{10′}, R^{11′}, R^{12′}, and R^{13′} are independently H, chloro, fluoro, C₁-C₆ alkyl, (CHO)C₁-C₆ alkyl, 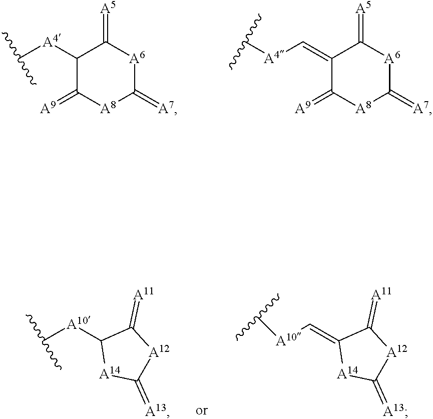
A⁴ is —(CH₂)ₚ—;
p is an integer of 1, 2, 3, 4, 5, or 6;
A⁴ is —(CH₂)_{q}—;
q is an integer of 1, 2, 3, 4, 5, or 6;
A⁵, A⁷, and A⁹ are independently O, S, or NH;
A⁶ and A⁸ are independently NH or CH₂;
A^{10′} is —(CH₂)ᵣ—;
r is an integer of 1, 2, 3, 4, 5, or 6;
A^{10″} is —(CH₂)ₜ—;
t is an integer of 1, 2, 3, 4, 5, or 6;
A¹¹ and A¹³ are independently O, S, or NH; and
A¹² and A¹⁴ are independently NH or CH₂.

11. The method of any one of claims 1 to 6, wherein R⁴ is H and R³ is —X—Y—R⁵.

12. The method of claim 11, wherein:
R⁵ is optionally substituted aryl, optionally substituted 3- to 9-membered heterocyclo, 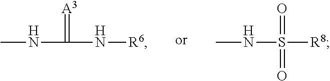
A³ is O, S, or NH;
R⁶ is optionally substituted C₁-C₆ alkyl or S(═O)₂R⁷;
R⁷ is optionally substituted C₁-C₆ alkyl or optionally substituted (3- to 6-membered heterocyclo)C₁-C₆ alkyl; and
R⁸ is optionally substituted (3- to 6-membered heterocyclo)C₁-C₆ alkyl.

13. The method of claim 12, wherein R⁵ is an optionally substituted 3- to 6-membered heterocyclo selected from the group consisting of:

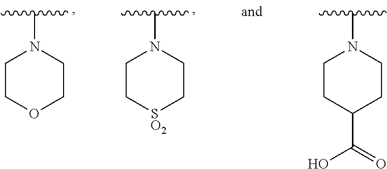 [SMILES: *N1CCOCC1 ; *N1CCS(=O)(=O)CC1 ; *N1CCC(C(=O)O)CC1]

14. The method of claim 11, wherein:
X is S, O, or NH;
n is an integer of 1, 2, or 3;
m is an integer of 1 or 2;
R⁵ is optionally substituted aryl, optionally substituted 9-membered heterocyclo, 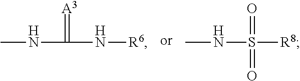
A³ is O;
R⁶ is S(═O)₂R⁷;
R⁷ is (morpholine)C₁-C₆ alkyl; and
R⁸ is optionally substituted (6-membered heterocyclo)C₁-C₆ alkyl.

15. The method of claim 14, wherein:
R⁵ is 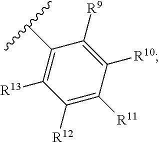
R⁹, R¹⁰, R¹¹, R¹², and R¹³ are independently H, chloro, fluoro, C₁-C₆ alkyl, or (CHO)C₁-C₆ alkyl.

16. The method of any one of claims 1 to 6, wherein R³ is —X—Y—R⁵ and R⁴ is —Y′—R⁵.

17. The method of claim 16, wherein:
Y′ is —(CH₂)_{n′}—,
n′ is 1; and
R^{5′} is optionally substituted aryl.

18. The method of claim 16 or 17, wherein:
R⁵ is 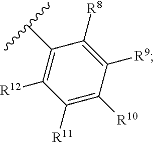
R⁸, R⁹, R¹⁰, R¹¹, and R¹² are independently H, chloro, fluoro, C₁-C₆ alkyl, or (CHO)C₁-C₆ alkyl.

19. The method of claim 16, wherein:
X is S;
Y is —(CH₂)ₙ—;
n is 2; and
R⁵ is optionally substituted 3- to 6-membered heterocyclo.

20. The method of claim 19, wherein R⁵ is selected from the group consisting of:

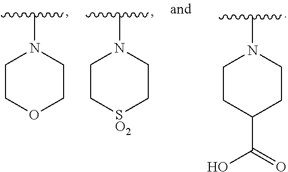

21. The method of claim 1, wherein the compound of Formula (Ia) is selected from the group consisting of:

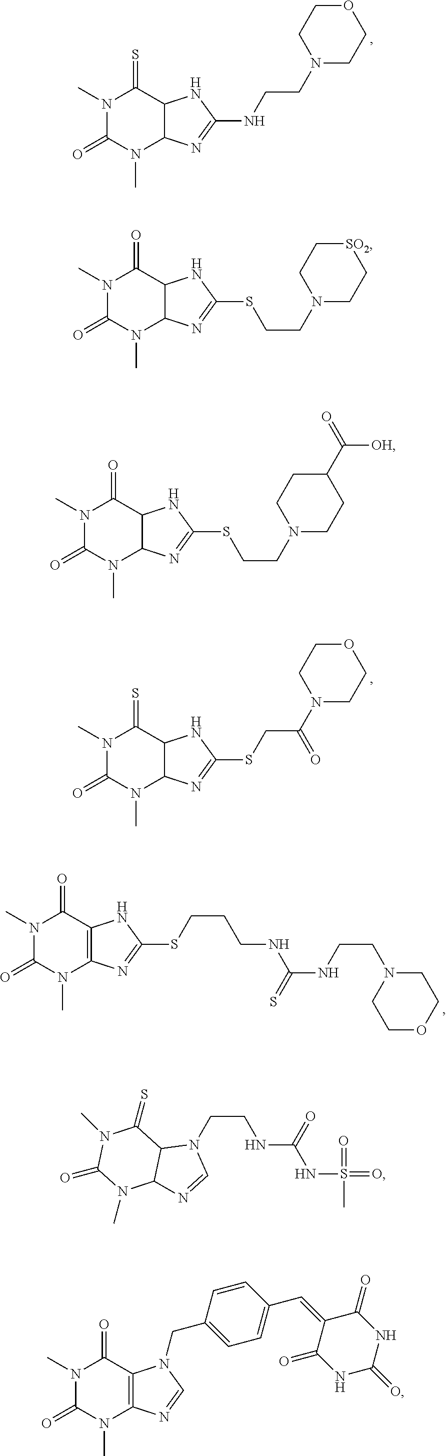

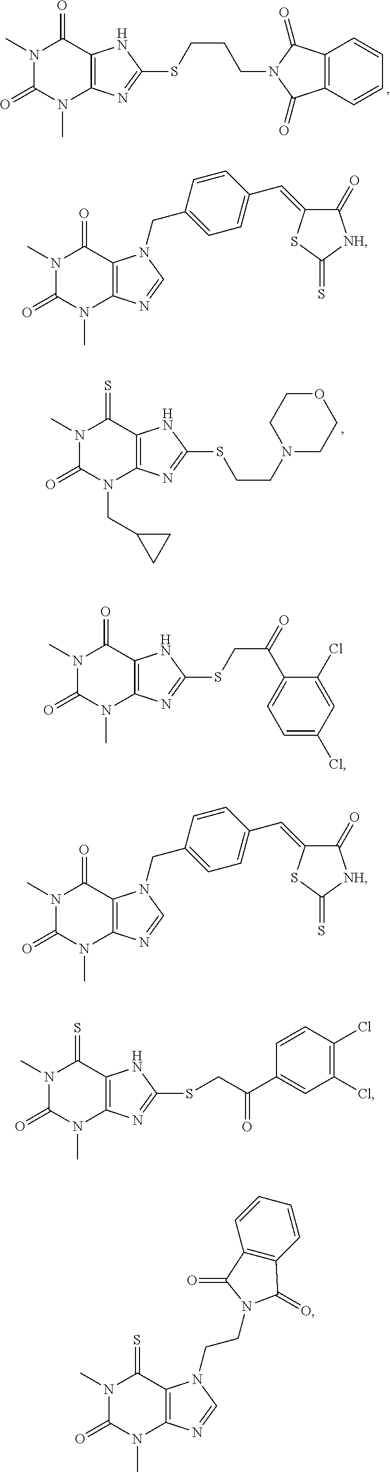

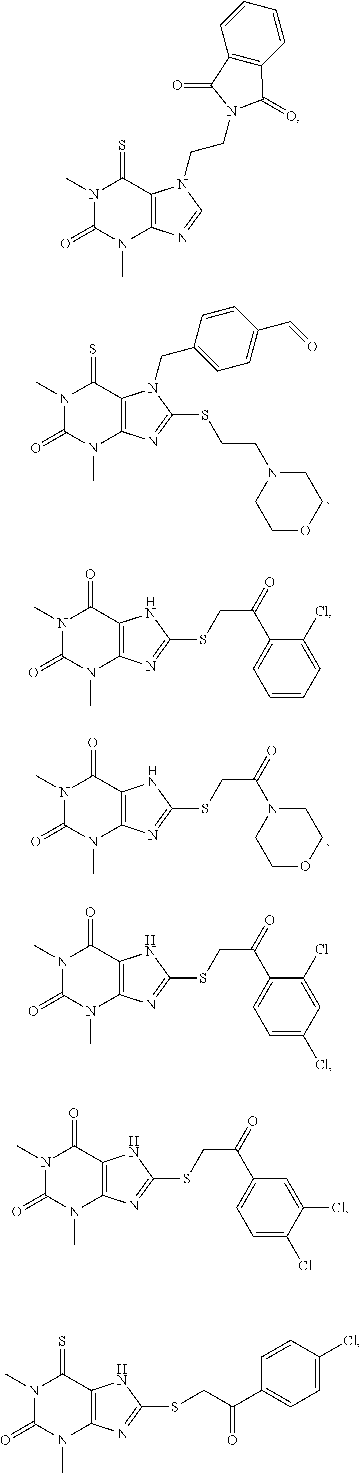

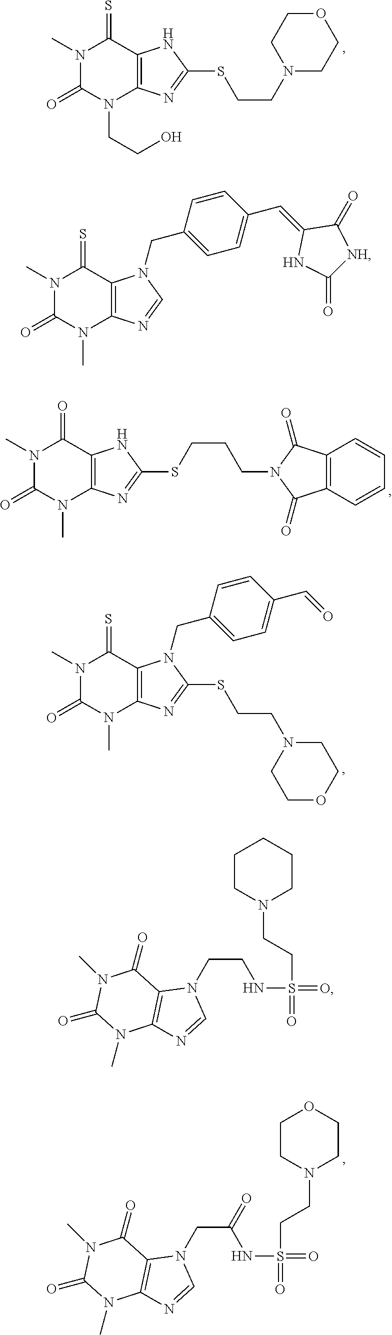

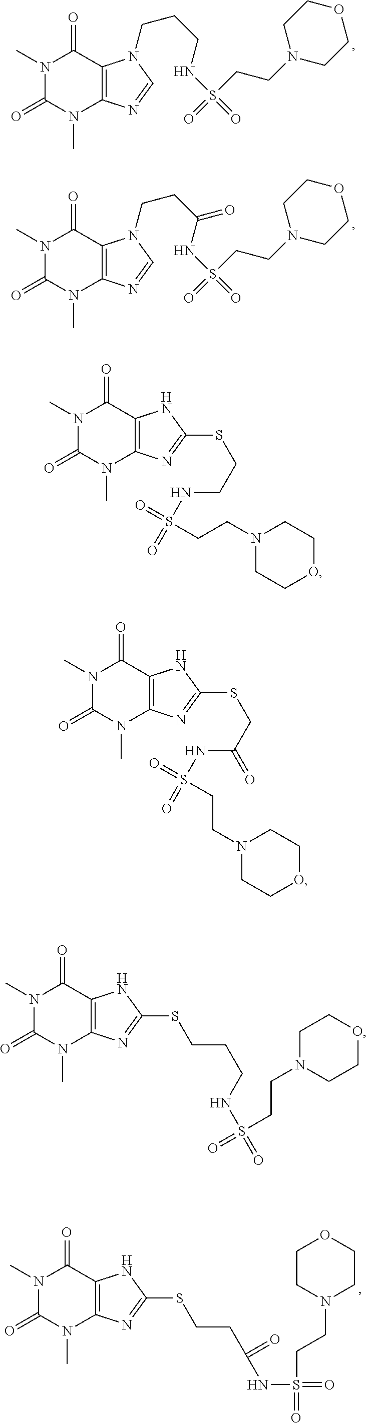 [SMILES: Cn1c(=O)c2c(ncn2CCCNS(=O)(=O)CCN2CCOCC2)n(C)c1=O ; Cn1c(=O)c2c(ncn2CCC(=O)NS(=O)(=O)CCN2CCOCC2)n(C)c1=O ; Cn1c(=O)c2[nH]c(SCCNS(=O)(=O)CCN3CCOCC3)nc2n(C)c1=O ; Cn1c(=O)c2[nH]c(SCC(=O)NS(=O)(=O)CCN3CCOCC3)nc2n(C)c1=O ; Cn1c(=O)c2[nH]c(SCCCNS(=O)(=O)CCN3CCOCC3)nc2n(C)c1=O ; Cn1c(=O)c2[nH]c(SCCC(=O)NS(=O)(=O)CCN3CCOCC3)nc2n(C)c1=O]

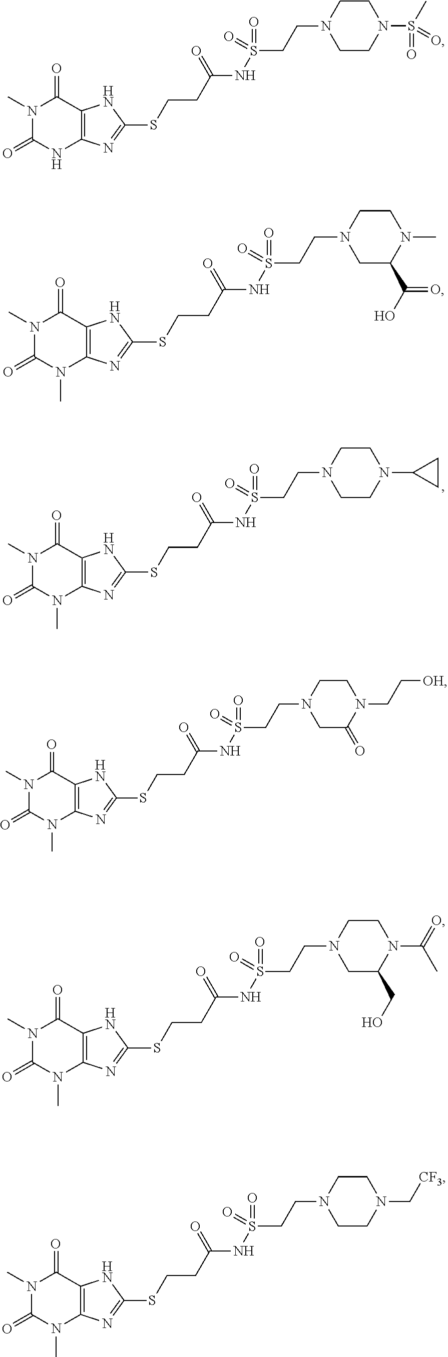 [SMILES: Cn1c(=O)[nH]c2nc(SCCC(=O)NS(=O)(=O)CCN3CCN(S(C)(=O)=O)CC3)[nH]c2c1=O ; CN1CCN(CCS(=O)(=O)NC(=O)CCSc2nc3c([nH]2)c(=O)n(C)c(=O)n3C)C[C@@H]1C(=O)O ; Cn1c(=O)c2[nH]c(SCCC(=O)NS(=O)(=O)CCN3CCN(C4CC4)CC3)nc2n(C)c1=O ; Cn1c(=O)c2[nH]c(SCCC(=O)NS(=O)(=O)CCN3CCN(CCO)C(=O)C3)nc2n(C)c1=O ; CC(=O)N1CCN(CCS(=O)(=O)NC(=O)CCSc2nc3c([nH]2)c(=O)n(C)c(=O)n3C)C[C@@H]1CO ; Cn1c(=O)c2[nH]c(SCCC(=O)NS(=O)(=O)CCN3CCN(CC(F)(F)F)CC3)nc2n(C)c1=O]

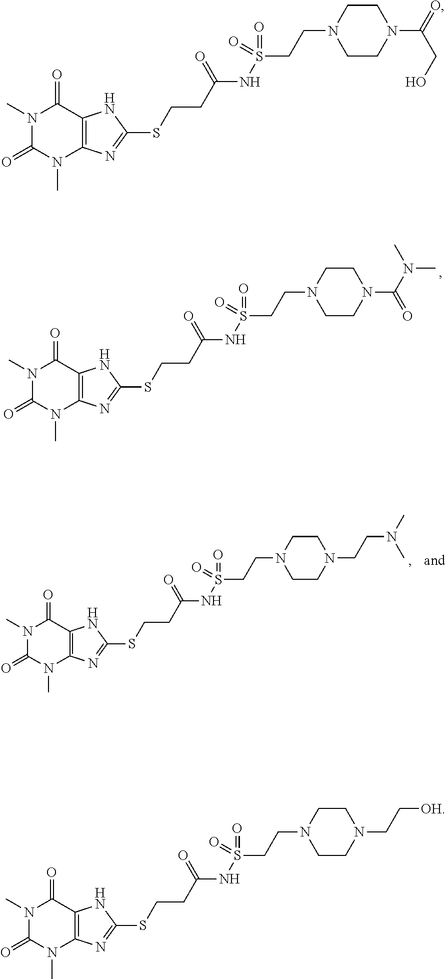 [SMILES: Cn1c(=O)c2[nH]c(SCCC(=O)NS(=O)(=O)CCN3CCN(C(=O)CO)CC3)nc2n(C)c1=O ; CN(C)C(=O)N1CCN(CCS(=O)(=O)NC(=O)CCSc2nc3c([nH]2)c(=O)n(C)c(=O)n3C)CC1 ; CN(C)CCN1CCN(CCS(=O)(=O)NC(=O)CCSc2nc3c([nH]2)c(=O)n(C)c(=O)n3C)CC1 ; Cn1c(=O)c2[nH]c(SCCC(=O)NS(=O)(=O)CCN3CCN(CCO)CC3)nc2n(C)c1=O]

22. The method of claim 1, wherein the compound of Formula (Ib) is selected from the group consisting of:

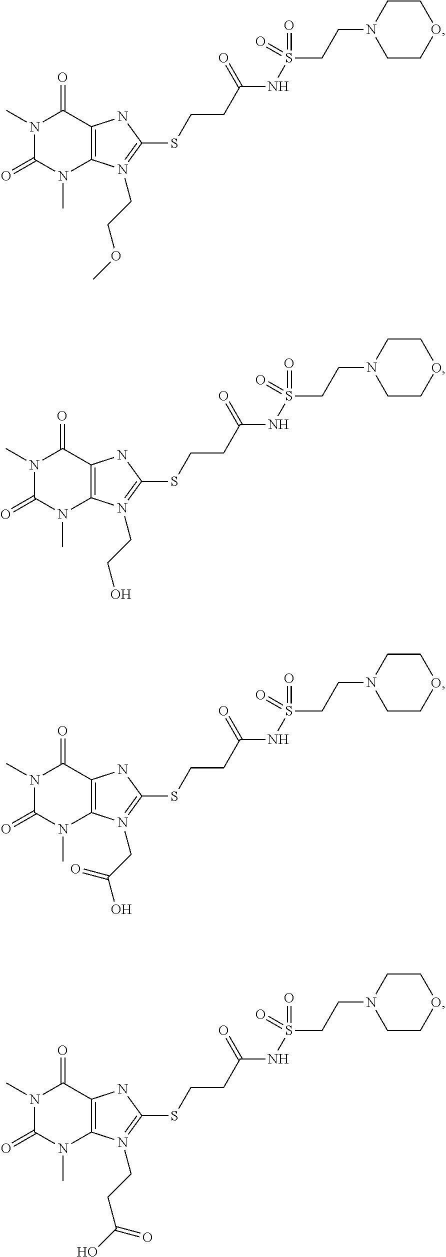

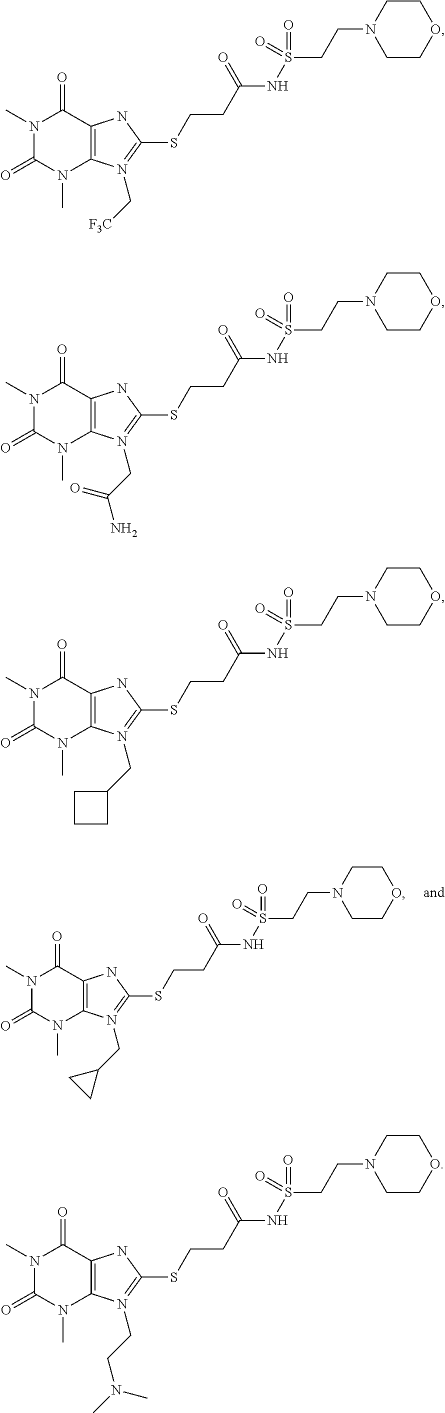

23. The method of any one of claims 1 to 22, wherein the coronavirus infection is caused by human coronavirus OC43 (HCoV-OC43), human coronavirus HKU1 (HCoV-HKU1), human coronavirus 229E (HCoV-229E), human coronavirus NL63 (HCoV-NL63), middle east respiratory syndrome-related coronavirus (MERS-CoV), severe acute respiratory syndrome coronavirus (SARS-CoV), or severe acute respiratory syndrome coronavirus 2 (SARS-CoV-2).

24. The method of any one of claims 1 to 23, wherein the compound of Formula (Ia) or Formula (Ib) is administered as part of a pharmaceutical composition comprising a pharmaceutical acceptable carrier, diluent, or excipient.

25. A compound of Formula (Ia) or Formula (Ib): 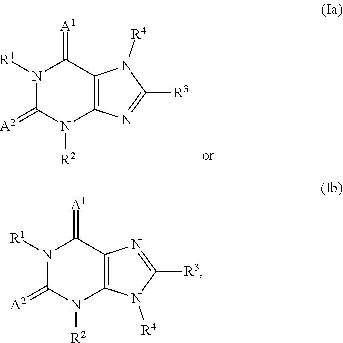
wherein:
A¹ and A² are independently O, S, or NH;
R¹ and R² are independently unsubstituted C₁-C₆ alkyl, (hydroxy)C₁-C₆ alkyl, or (C₃-C₆ cycloalkyl)C₁-C₆ alkyl; and
R³ is H or —X—Y—R⁵;
R⁴ is H or —Y′—R^{5′};
X is absent, S, O, or NH;
Y is —(CH₂)ₙ— or —(CH₂)ₘ(C═O)—;
n is an integer of 1, 2, 3, 4, 5, or 6;
m is an integer of 0, 1, 2, 3, 4, 5, or 6;
Y′ is —(CH₂)_{n′}— or —(CH₂)_{m′}(C═O)—;
n′ is an integer of 1, 2, 3, 4, 5, or 6;
m′ is an integer of 0, 1, 2, 3, 4, 5, or 6;
R⁵ is optionally substituted aryl, optionally substituted 3- to 10-membered heterocyclo, 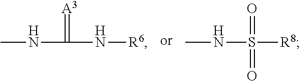
A³ is O, S, or NH;
R⁶ is optionally substituted C₁-C₆ alkyl or S(═O)₂R⁷;
R⁷ is optionally substituted C₁-C₆ alkyl, or optionally substituted (3- to 10-membered heterocyclo)C₁-C₆ alkyl;
R⁸ is optionally substituted (3- to 6-membered heterocyclo)C₁-C₆ alkyl;
R^{5′} is C₁-C₄ haloalkyl, hydroxy, C₃-C₆ cycloalkyl, C₁-C₄ alkoxy, amino, optionally substituted aryl, optionally substituted 3- to 10-membered heterocyclo, 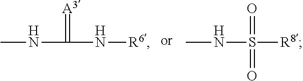
A^{3′} is O, S, or NH;
R^{6′} is optionally substituted C₁-C₆ alkyl or S(═O)₂R⁷;
R^{7′} is optionally substituted C₁-C₆ alkyl, or optionally substituted (3- to 10-membered heterocyclo)C₁-C₆ alkyl; and
R^{8′} is optionally substituted (3- to 6-membered heterocyclo)C₁-C₆ alkyl;
or a pharmaceutically acceptable salt thereof,
with the proviso that R³ and R⁴ are not both H.

26. The compound of claim 25, wherein the compound is a compound of Formula (Ia).

27. The compound of claim 25, wherein the compound is a compound of Formula (Ib).

28. The compound of any one of claims 25 to 27, wherein A¹ and A² are independently O or S.

29. The compound of any one of claims 25 to 28, wherein R¹ and R² are independently unsubstituted C₁-C₆ alkyl.

30. The compound of any one of claims 25 to 29, wherein R¹ and R² are methyl.

31. The compound of any one of claims 25 to 30, wherein R³ is H and R⁴ is —Y′—R⁵.

32. The compound of any one of claims 25 to 31, wherein
Y′ is —(CH₂)_{n′}—,
n′ is an integer of 1 or 2;
R^{5′} is optionally substituted aryl or 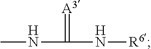
A^{3′} is O, S, or NH;
R^{6′} is S(═O)₂R^{7′}; and
R^{7′} is optionally substituted C₁-C₆ alkyl or optionally substituted (3- to 6-membered heterocyclo)C₁-C₆ alkyl.

33. The compound of claim 32, wherein:
R^{5′} is 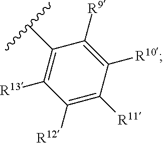
R^{9′}, R^{10′}, R^{11′}, R^{12′}, and R¹³ are independently H, chloro, fluoro, C₁-C₆ alkyl, (CHO)C₁-C₆ alkyl, 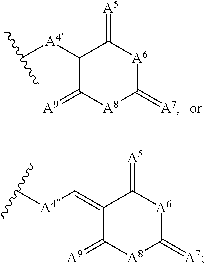
A⁴ is —(CH₂)ₚ—;
p is an integer of 1, 2, 3, 4, 5, or 6;
A⁴ is —(CH₂)_{q}—;
q is an integer of 1, 2, 3, 4, 5, or 6;
A⁴ is C₁-C₆ alkyl;
A⁵, A⁷, and A⁹ are independently O, S, or NH; and
A⁶ and A⁸ are independently NH or CH₂.

34. The compound of claim 32, wherein:
R^{5′} is 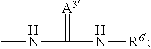
A³ is O;
R^{6′} is S(═O)₂R^{7′}; and
R^{7′} is C₁-C₆ alkyl.

35. The compound of any one of claims 25 to 34, wherein R³ is —X—Y—R⁵ and R⁴ is H.

36. The compound of claim 35, wherein:
X is S or NH;
Y is —(CH₂)ₙ— or —(CH₂)ₘ(C═O)—;
n is an integer of 1 or 2;
m is an integer of 0, 1, or 2;
R⁵ is optionally substituted 3- to 10-membered heterocyclo or 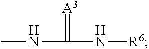
A³ is O, S, or NH; and
R⁶ is optionally substituted (3- to 6-membered heterocyclo)C₁-C₆ alkyl.

37. The compound of claim 36, wherein:
R⁵ is 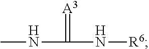
A³ is S; and
R⁶ is (morpholine)C₁-C₆alkyl.

38. The compound of claim 37, wherein R⁵ is selected from the group consisting of:

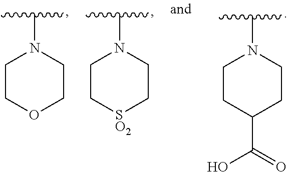

39. The compound of claim 35, wherein:
Y is —(CH₂)ₘ(C═O)—;
m is 2;
R⁵ is: 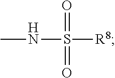
and
R⁸ is optionally substituted (6-membered heterocyclo)C₁-C₃ alkyl.

40. The compound of claim 39, wherein R⁸ is:

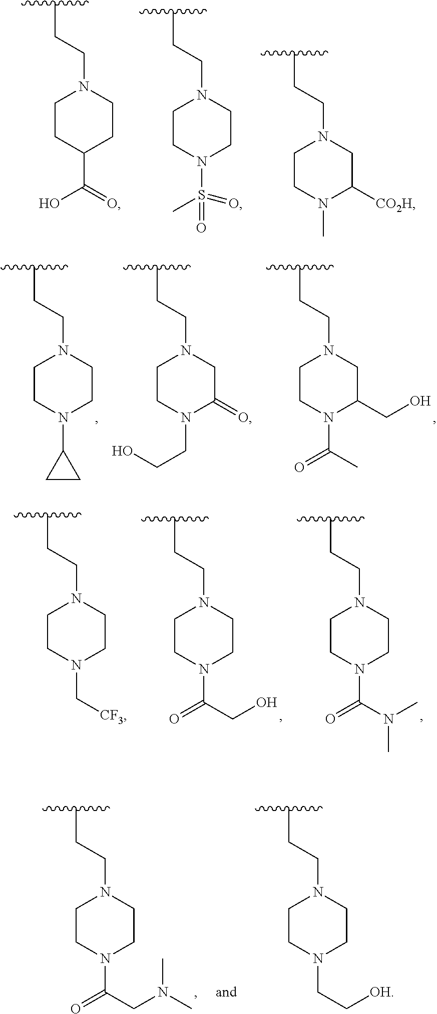

41. The compound of claim 25, wherein the compound of Formula (Ia) is selected from the group consisting of:

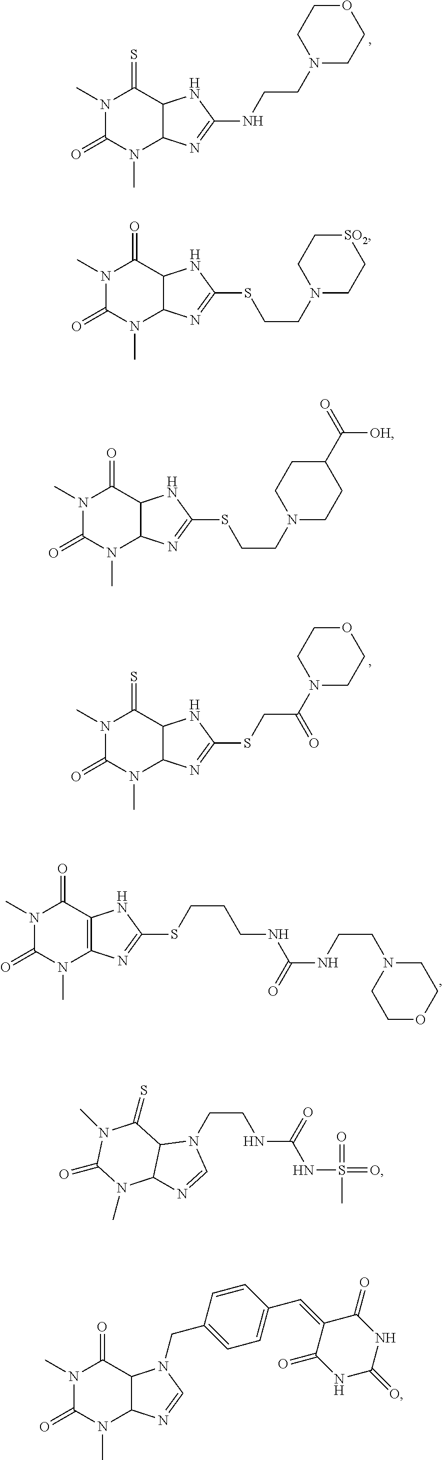

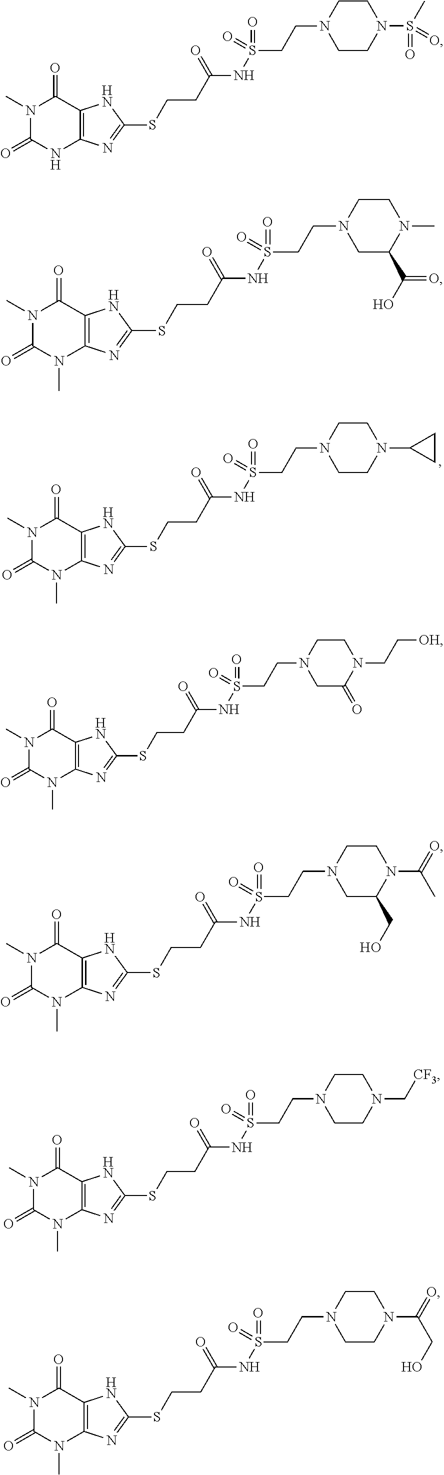

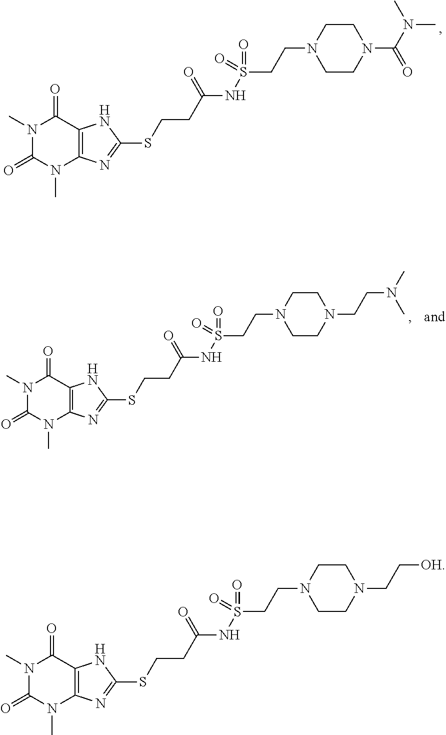 [SMILES: CN(C)C(=O)N1CCN(CCS(=O)(=O)NC(=O)CCSc2nc3c([nH]2)c(=O)n(C)c(=O)n3C)CC1 ; CN(C)CCN1CCN(CCS(=O)(=O)NC(=O)CCSc2nc3c([nH]2)c(=O)n(C)c(=O)n3C)CC1 ; Cn1c(=O)c2[nH]c(SCCC(=O)NS(=O)(=O)CCN3CCN(CCO)CC3)nc2n(C)c1=O]

42. The compound of claim 25, wherein the compound of Formula (Ib) is selected from the group consisting of:

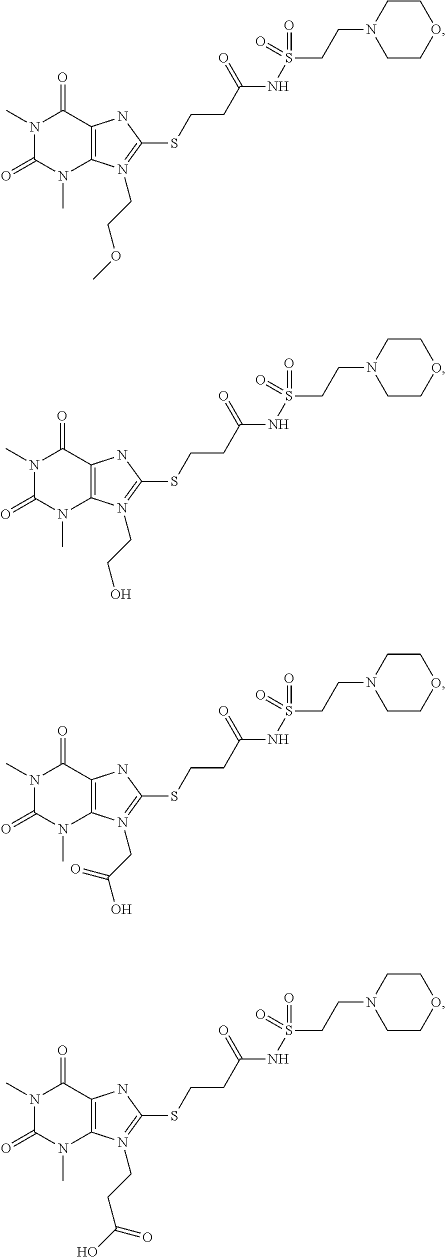

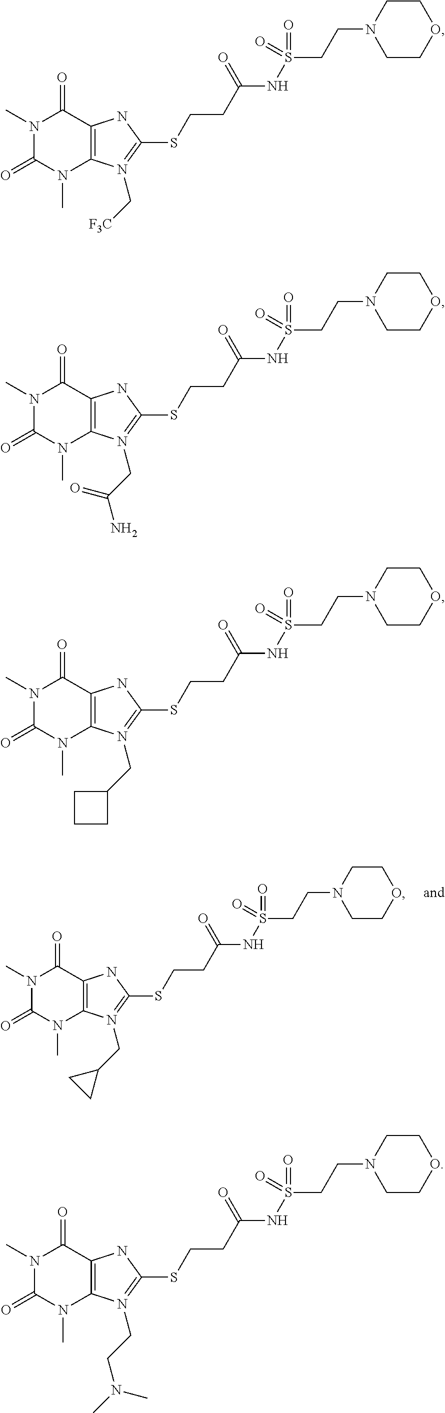

43. A pharmaceutical composition comprising the compound of any one of claims 25 to 42 and a pharmaceutically acceptable carrier, diluent, or excipient.

44. A method for treating or reducing the symptoms associated with a coronavirus infection, the method comprising administering to a subject in need thereof a therapeutically effective amount of a compound of any one of claims 25 to 42 or the composition of claim 43.

45. A method for inhibiting coronavirus pathogenicty and symptoms by administering to a subject in need thereof a therapeutically effective amount of a compound that is chemically complementary to the Mac1 channel and interactions defined and implied by the crystal structures of the Mac1/PARG-345 (MES8-4) complex and the Mac1/PARG-329 complex with their neighboring bound water molecules.
